SEQUENCE LISTING (74 sequences)

SEQ ID NO: 1 - DNA, 19 bp, Artificial Sequence
  Synthesized sequence
ggcacttgctcatgcacct

SEQ ID NO: 2 - DNA, 22 bp, Artificial Sequence
  Synthetic sequence
ggatggagagacagagctggtt

SEQ ID NO: 3 - DNA, 22 bp, Artificial Sequence
  Synthetic sequence
tacggtgcagctgactccatat

SEQ ID NO: 4 - DNA, 21 bp, Artificial Sequence
  Synthetic sequence
ggcagagcacaactgttcctt

SEQ ID NO: 5 - DNA, 21 bp, Artificial Sequence
  Synthetic sequence
gagatctccgagatgccttca

SEQ ID NO: 6 - DNA, 26 bp, Artificial Sequence
  Synthetic sequence
caaggactcctttaacaacaagttgt

SEQ ID NO: 7 - DNA, 20 bp, Artificial Sequence
  Synthetic sequence
agatcccacacgccacattc

SEQ ID NO: 8 - DNA, 20 bp, Artificial Sequence
  Synthetic sequence
tgcggaaacctctcttgcat

SEQ ID NO: 9 - DNA, 22 bp, Artificial Sequence
  Synthetic sequence
gaacaactgagggaaccaaacc

SEQ ID NO: 10 - DNA, 21 bp, Artificial Sequence
  Synthetic sequence
gcagcaacagcagcattacag

SEQ ID NO: 11 - DNA, 25 bp, Artificial Sequence
  Synthetic sequence
tccatccagtgctacttgtgtttac

SEQ ID NO: 12 - DNA, 22 bp, Artificial Sequence
  Synthetic sequence
cactgaaacagcccaaaatgaa

SEQ ID NO: 13 - DNA, 2390 bp, Homo sapiens
agagccttcgtttgccaagtcgcctccagaccgcagacatgaaacttgtcttcctcgtcctgctgttcctcggggccctcggactgtgtctggctggccgtaggaggagtgttcagtggtgcgccgtatcccaacccgaggccacaaaatgcttccaatggcaaaggaatatgagaaaagtgcgtggccctcctgtcagctgcataaagagagactcccccatccagtgtatccaggccattgcggaaaacagggccgatgctgtgacccttgatggtggtttcatatacgaggcaggcctggccccctacaaactgcgacctgtagcggcggaagtctacgggaccgaaagacagccacgaactcactattatgccgtggctgtggtgaagaagggcggcagctttcagctgaacgaactgcaaggtctgaagtcctgccacacaggccttcgcaggaccgctggatggaatgtccctatagggacacttcgtccattcttgaattggacgggtccacctgagcccattgaggcagctgtggccaggttcttctcagccagctgtgttcccggtgcagataaaggacagttccccaacctgtgtcgcctgtgtgcggggacaggggaaaacaaatgtgccttctcctcccaggaaccgtacttcagctactctggtgccttcaagtgtctgagagacggggctggagacgtggcttttatcagagagagcacagtgtttgaggacctgtcagacgaggctgaaagggacgagtatgagttactctgcccagacaacactcggaagccagtggacaagttcaaagactgccatctggcccgggtcccttctcatgccgttgtggcacgaagtgtgaatggcaaggaggatgccatctggaatcttctccgccaggcacaggaaaagtttggaaaggacaagtcaccgaaattccagctctttggctcccctagtgggcagaaagatctgctgttcaaggactctgccattgggttttcgagggtgcccccgaggatagattctgggctgtaccttggctccggctacttcactgccatccagaacttgaggaaaagtgaggaggaagtggctgcccggcgtgcgcgggtcgtgtggtgtgcggtgggcgagcaggagctgcgcaagtgtaaccagtggagtggcttgagcgaaggcagcgtgacctgctcctcggcctccaccacagaggactgcatcgccctggtgctgaaaggagaagctgatgccatgagtttggatggaggatatgtgtacactgcaggcaaatgtggtttggtgcctgtcctggcagagaactacaaatcccaacaaagcagtgaccctgatcctaactgtgtggatagacctgtggaaggatatcttgctgtggcggtggttaggagatcagacactagccttacctggaactctgtgaaaggcaagaagtcctgccacaccgccgtggacaggactgcaggctggaatatccccatgggcctgctcttcaaccagacgggctcctgcaaatttgatgaatatttcagtcaaagctgtgcccctgggtctgacccgagatctaatctctgtgctctgtgtattggcgacgagcagggtgagaataagtgcgtgcccaacagcaacgagagatactacggctacactggggctttccggtgcctggctgagaatgctggagacgttgcatttgtgaaagatgtcactgtcttgcagaacactgatggaaataacaatgaggcatgggctaaggatttgaagctggcagactttgcgctgctgtgcctcgatggcaaacggaagcctgtgactgaggctagaagctgccatcttgccatggccccgaatcatgccgtggtgtctcggatggataaggtggaacgcctgaaacaggtgttgctccaccaacaggctaaatttgggagaaatggatctgactgcccggacaagttttgcttattccagtctgaaaccaaaaaccttctgttcaatgacaacactgagtgtctggccagactccatggcaaaacaacatatgaaaaatatttgggaccacagtatgtcgcaggcattactaatctgaaaaagtgctcaacctcccccctcctggaagcctgtgaattcctcaggaagtaaaaccgaagaagatggcccagctccccaagaaagcctcagccattcactgcccccagctcttctccccaggtgtgttggggccttggcctcccctgctgaaggtggggattgcccatccatctgcttacaattccctgctgtcgtcttagcaagaagtaaaatgagaaattttgttgatattctctccttaaaaaaaaaaaaaaaaaaaaaaaaaaaaaa

SEQ ID NO: 14 - DNA, 1437 bp, Homo sapiens
cagggccgagccagccccttcaccaccagccggccgcgccccgggaagggaagtttgtggcggaggaggttcgtacgggaggagggggaggcgcccacgcatctggggctgactcgctctttcgcaaaacgtctgggaggagtccctggggccacaaaactgcctccttcctgaggccagaaggagagaagacgtgcagggaccccgcgcacaggagctgccctcgcgacatgggtcacccgccgctgctgccgctgctgctgctgctccacacctgcgtcccagcctcttggggcctgcggtgcatgcagtgtaagaccaacggggattgccgtgtggaagagtgcgccctgggacaggacctctgcaggaccacgatcgtgcgcttgtgggaagaaggagaagagctggagctggtggagaaaagctgtacccactcagagaagaccaacaggaccctgagctatcggactggcttgaagatcaccagccttaccgaggttgtgtgtgggttagacttgtgcaaccagggcaactctggccgggctgtcacctattcccgaagccgttacctcgaatgcatttcctgtggctcatcagacatgagctgtgagaggggccggcaccagagcctgcagtgccgcagccctgaagaacagtgcctggatgtggtgacccactggatccaggaaggtgaagaagggcgtccaaaggatgaccgccacctccgtggctgtggctaccttcccggctgcccgggctccaatggtttccacaacaacgacaccttccacttcctgaaatgctgcaacaccaccaaatgcaacgagggcccaatcctggagcttgaaaatctgccgcagaatggccgccagtgttacagctgcaaggggaacagcacccatggatgctcctctgaagagactttcctcattgactgccgaggccccatgaatcaatgtctggtagccaccggcactcacgaacgctcactctggggaagctggttgccatgtaaaagtactactgccctgagaccaccatgctgtgaggaagcccaagctactcatgtataaatgccatgtggagatagagccccagatgtttcagccatctcagcccaggcaccagacaagtgggtgaagaagccaccttggacatgtagccccagcagatgtgatatagagaagaaacaggaaacttggctatattagtttcctagggctgcctgtgataaattattacaaactttataaactaacacattgtgtgcctatatcaaaacatcatggaaggacaggcacagtggctcatgcctgtagtcctagcactttgggagggtgagaaaggaagatctcttgagctcaggagttcaagatcagcctgggcaacacagtgagacctcatctccactaaaaataaaaaaaaattggctgg

SEQ ID NO: 15 - DNA, 1413 bp, Homo sapiens
cagggccgagccagccccttcaccaccagccggccgcgccccgggaagggaagtttgtggcggaggaggttcgtacgggaggagggggaggcgcccacgcatctggggctgactcgctctttcgcaaaacgtctgggaggagtccctggggccacaaaactgcctccttcctgaggccagaaggagagaagacgtgcagggaccccgcgcacaggagctgccctcgcgacatgggtcacccgccgctgctgccgctgctgctgctgctccacacctgcgtcccagcctcttggggcctgcggtgcatgcagtgtaagaccaacggggattgccgtgtggaagagtgcgccctgggacaggacctctgcaggaccacgatcgtgcgcttgtgggaagaaggagaagagctggagctggtggagaaaagctgtacccactcagagaagaccaacaggaccctgagctatcggactggcttgaagatcaccagccttaccgaggttgtgtgtgggttagacttgtgcaaccagggcaactctggccgggctgtcacctattcccgaagccgttacctcgaatgcatttcctgtggctcatcagacatgagctgtgagaggggccggcaccagagcctgcagtgccgcagccctgaagaacagtgcctggatgtggtgacccactggatccaggaaggtgaagaagtcctggagcttgaaaatctgccgcagaatggccgccagtgttacagctgcaaggggaacagcacccatggatgctcctctgaagagactttcctcattgactgccgaggccccatgaatcaatgtctggtagccaccggcactcacgaaccgaaaaaccaaagctatatggtaagaggctgtgcaaccgcctcaatgtgccaacatgcccacctgggtgacgccttcagcatgaaccacattgatgtctcctgctgtactaaaagtggctgtaaccacccagacctggatgtccagtaccgcagtggggctgctcctcagcctggccctgcccatctcagcctcaccatcaccctgctaatgactgccagactgtggggaggcactctcctctggacctaaacctgaaatccccctctctgccctggctggatccgggggacccctttgcccttccctcggctcccagccctacagacttgctgtgtgacctcaggccagtgtgccgacctctctgggcctcagttttcccagctatgaaaacagctatctcacaaagttgtgtgaagcagaagagaaaagctggaggaaggccgtgggccaatgggagagctcttgttattattaatattgttgccgctgttgtgttgttgttattaattaatattcatattatttattttatacttacataaagattttgtaccagtgg

SEQ ID NO: 16 - DNA, 1548 bp, Homo sapiens
cagggccgagccagccccttcaccaccagccggccgcgccccgggaagggaagtttgtggcggaggaggttcgtacgggaggagggggaggcgcccacgcatctggggctgactcgctctttcgcaaaacgtctgggaggagtccctggggccacaaaactgcctccttcctgaggccagaaggagagaagacgtgcagggaccccgcgcacaggagctgccctcgcgacatgggtcacccgccgctgctgccgctgctgctgctgctccacacctgcgtcccagcctcttggggcctgcggtgcatgcagtgtaagaccaacggggattgccgtgtggaagagtgcgccctgggacaggacctctgcaggaccacgatcgtgcgcttgtgggaagaaggagaagagctggagctggtggagaaaagctgtacccactcagagaagaccaacaggaccctgagctatcggactggcttgaagatcaccagccttaccgaggttgtgtgtgggttagacttgtgcaaccagggcaactctggccgggctgtcacctattcccgaagccgttacctcgaatgcatttcctgtggctcatcagacatgagctgtgagaggggccggcaccagagcctgcagtgccgcagccctgaagaacagtgcctggatgtggtgacccactggatccaggaaggtgaagaagggcgtccaaaggatgaccgccacctccgtggctgtggctaccttcccggctgcccgggctccaatggtttccacaacaacgacaccttccacttcctgaaatgctgcaacaccaccaaatgcaacgagggcccaatcctggagcttgaaaatctgccgcagaatggccgccagtgttacagctgcaaggggaacagcacccatggatgctcctctgaagagactttcctcattgactgccgaggccccatgaatcaatgtctggtagccaccggcactcacgaaccgaaaaaccaaagctatatggtaagaggctgtgcaaccgcctcaatgtgccaacatgcccacctgggtgacgccttcagcatgaaccacattgatgtctcctgctgtactaaaagtggctgtaaccacccagacctggatgtccagtaccgcagtggggctgctcctcagcctggccctgcccatctcagcctcaccatcaccctgctaatgactgccagactgtggggaggcactctcctctggacctaaacctgaaatccccctctctgccctggctggatccgggggacccctttgcccttccctcggctcccagccctacagacttgctgtgtgacctcaggccagtgtgccgacctctctgggcctcagttttcccagctatgaaaacagctatctcacaaagttgtgtgaagcagaagagaaaagctggaggaaggccgtgggccaatgggagagctcttgttattattaatattgttgccgctgttgtgttgttgttattaattaatattcatattatttattttatacttacataaagattttgtaccagtgg

SEQ ID NO: 17 - DNA, 3150 bp, Homo sapiens
aaaagtttcttttctttgaatgacagaactacagcataatgcgtggcttcaacctgctcctcttctggggatgttgtgttatgcacagctgggaagggcacataagacccacacggaaaccaaacacaaagggtaataactgtagagacagtaccttgtgcccagcttatgccacctgcaccaatacagtggacagttactattgcgcttgcaaacaaggcttcctgtccagcaatgggcaaaatcacttcaaggatccaggagtgcgatgcaaagatattgatgaatgttctcaaagcccccagccctgtggtcctaactcatcctgcaaaaacctgtcagggaggtacaagtgcagctgtttagatggtttctcttctcccactggaaatgactgggtcccaggaaagccgggcaatttctcctgtactgatatcaatgagtgcctcaccagcagcgtctgccctgagcattctgactgtgtcaactccatgggaagctacagttgcagctgtcaagttggattcatctctagaaactccacctgtgaagacgtggatgaatgtgcagatccaagagcttgcccagagcatgcaacttgtaataacactgttggaaactactcttgtttctgcaacccaggatttgaatccagcagtggccacttgagtttccagggtctcaaagcatcgtgtgaagatattgatgaatgcactgaaatgtgccccatcaattcaacatgcaccaacactcctgggagctacttttgcacctgccaccctggctttgcaccaagcaatggacagttgaatttcacagaccaaggagtggaatgtagagatattgatgagtgccgccaagatccatcaacctgtggtcctaattctatctgcaccaatgccctgggctcctacagctgtggctgcattgcaggctttcatcccaatccagaaggctcccagaaagatggcaacttcagctgccaaagggttctcttcaaatgtaaggaagatgtgatacccgataataagcagatccagcaatgccaagagggaaccgcagtgaaacctgcatatgtctccttttgtgcacaaataaataacatcttcagcgttctggacaaagtgtgtgaaaataaaacgaccgtagtttctctgaagaatacaactgagagctttgtccctgtgcttaaacaaatatccacgtggactaaattcaccaaggaagagacgtcctccctggccacagtcttcctggagagtgtggaaagcatgacactggcatctttttggaaaccctcagcaaatatcactccggctgttcggacggaatacttagacattgagagcaaagttatcaacaaagaatgcagtgaagagaatgtgacgttggacttggtagccaagggggataagatgaagatcgggtgttccacaattgaggaatctgaatccacagagaccactggtgtggcttttgtctcctttgtgggcatggaatcggttttaaatgagcgcttcttcaaagaccaccaggctcccttgaccacctctgagatcaagctgaagatgaattctcgagtcgttgggggcataatgactggagagaagaaagacggcttctcagatccaatcatctacactctggagaacattcagccaaagcagaagtttgagaggcccatctgtgtttcctggagcactgatgtgaagggtggaagatggacatcctttggctgtgtgatcctggaagcttctgagacatataccatctgcagctgtaatcagatggcaaatcttgccgttatcatggcgtctggggagctcacgatggacttttccttgtacatcattagccatgtaggcattatcatctccttggtgtgcctcgtcttggccatcgccacctttctgctgtgtcgctccatccgaaatcacaacacctacctccacctgcacctctgcgtgtgtctcctcttggcgaagactctcttcctcgccggtatacacaagactgacaacaagatgggctgcgccatcatcgcgggcttcctgcactaccttttccttgcctgcttcttctggatgctggtggaggctgtgatactgttcttgatggtcagaaacctgaaggtggtgaattacttcagctctcgcaacatcaagatgctgcacatctgtgcctttggttatgggctgccgatgctggtggtggtgatctctgccagtgtgcagccacagggctatggaatgcataatcgctgctggctgaatacagagacagggttcatctggagtttcttggggccagtttgcacagttatagtgatcaactcccttctcctgacctggaccttgtggatcctgaggcagaggctttccagtgttaatgccgaagtctcaacgctaaaagacaccaggttactgaccttcaaggcctttgcccagctcttcatcctgggctgctcctgggtgctgggcatttttcagattggacctgtggcaggtgtcatggcttacctgttcaccatcatcaacagcctgcagggggccttcatcttcctcatccactgtctgctcaacggccaggtacgagaagaatacaagaggtggatcactgggaagacgaagcccagctcccagtcccagacctcaaggatcttgctgtcctccatgccatccgcttccaagacgggttaaagtcctttcttgctttcaaatatgctatggagccacagttgaggacagtagtttcctgcaggagcctaccctgaaatctcttctcagcttaacatggaaatgaggatcccaccagccccagaaccctctggggaagaatgttgggggcggtcttcctgtggttgtatgcactgatgagaaatcaggcgtttctgctccaaacgaccattttatcttcgtgctctgcaacttcttcaattccagagtttctgagaacagacccaaattcaatggcatgaccaagaacacctggctaccattttgttttctcctgcccttgttggtgcatggttctaagcatgcccctccagagcctatcatacgcctgatacagagaacctctcaataaatgatttgtcgcctgtctgactgatttaccctaggaaaaaaaaaaaaaaaaaaaaaaaaaaaa

SEQ ID NO: 18 - DNA, 2406 bp, Homo sapiens
gattctgtgtgtgtcctcagatgctcagccacagacctttgagggagtaaagggggcagacccacccaccttgcctccaggctctttccttcctggtcctgttctatggtggggctcccttgccagacttcagactgagaagtcagatgaagtttcaagaaaaggaaattggtgggtgacagagatgggtggaggggctggggaaaggctgtttacttcctcctgtctagtcggtttggtccctttagggctccggatatctttggtgacttgtccactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 19 - DNA, 1641 bp, Homo sapiens
ctccctgtgttggtggaggatgtctgcagcagcatttaaattctgggagggcttggttgtcagcagcagcaggaggaggcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagaatgctgtgtcctctgaagaaaccaatgactttaaacaagagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaatttggtggtgtcaattgcttatttgttttcccacggttgtccagcaattaataaaacataaccttttttactgcctaaaaaaaaaaaaaaaaa

SEQ ID NO: 20 - DNA, 1560 bp, Homo sapiens
ctccctgtgttggtggaggatgtctgcagcagcatttaaattctgggagggcttggttgtcagcagcagcaggaggaggcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagaatgctgtgtcctctgaagaaaccaatgactttaaacaagagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaatttggtggtgtcaattgcttatttgttttcccacggttgtccagcaattaataaaacataaccttttttactgcctaaaaaaaaaaaaaaaaa

SEQ ID NO: 21 - DNA, 1796 bp, Homo sapiens
ctgatggtatctctgtttcaggagtggtgacgcctaagctatcactggacatatcaaggacttcactaaattagcaggtaccactggtcttcttgtgcttatccgggcaagaacttatcgaaatacaatagaagtttttacttagaagagattttcagcagatgagaagctggtaacagagaccaaaatagtttggagactaaagaatcattgcacatttcactgctgagttgtattggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaatacaataagttaactattttattttctaatgtgcctagttctttccctgcttaatgaaaagcttgttttttcagtgtgaataaataatcgtaagcaaca

SEQ ID NO: 22 - DNA, 840 bp, Homo sapiens
actcgccacctcctcttccacccctgccaggcccagcagccaccacagcgcctgcttcctcggccctgaaatcatgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggctgagtgcacaggtgccgccagctgccgcaccagcccgaacaccattgagggagctgggagaccctccccacagtgccacccatgcagctgctccccaggccaccccgctgatggagccccaccttgtctgctaaataaacatgtgccctcaggccaaaaaaaaaaaaaaaaaa

SEQ ID NO: 23 - DNA, 4742 bp, Homo sapiens
ttttctgcccttctttgctttggtggcttccttgtggttcctcagtggtgcctgcaacccctggttcacctccttccaggttctggctccttccagccatggctctcagagtccttctgttaacagccttgaccttatgtcatgggttcaacttggacactgaaaacgcaatgaccttccaagagaacgcaaggggcttcgggcagagcgtggtccagcttcagggatccagggtggtggttggagccccccaggagatagtggctgccaaccaaaggggcagcctctaccagtgcgactacagcacaggctcatgcgagcccatccgcctgcaggtccccgtggaggccgtgaacatgtccctgggcctgtccctggcagccaccaccagcccccctcagctgctggcctgtggtcccaccgtgcaccagacttgcagtgagaacacgtatgtgaaagggctctgcttcctgtttggatccaacctacggcagcagccccagaagttcccagaggccctccgagggtgtcctcaagaggatagtgacattgccttcttgattgatggctctggtagcatcatcccacatgactttcggcggatgaaggagtttgtctcaactgtgatggagcaattaaaaaagtccaaaaccttgttctctttgatgcagtactctgaagaattccggattcactttaccttcaaagagttccagaacaaccctaacccaagatcactggtgaagccaataacgcagctgcttgggcggacacacacggccacgggcatccgcaaagtggtacgagagctgtttaacatcaccaacggagcccgaaagaatgcctttaagatcctagttgtcatcacggatggagaaaagtttggcgatcccttgggatatgaggatgtcatccctgaggcagacagagagggagtcattcgctacgtcattggggtgggagatgccttccgcagtgagaaatcccgccaagagcttaataccatcgcatccaagccgcctcgtgatcacgtgttccaggtgaataactttgaggctctgaagaccattcagaaccagcttcgggagaagatctttgcgatcgagggtactcagacaggaagtagcagctcctttgagcatgagatgtctcaggaaggcttcagcgctgccatcacctctaatggccccttgctgagcactgtggggagctatgactgggctggtggagtctttctatatacatcaaaggagaaaagcaccttcatcaacatgaccagagtggattcagacatgaatgatgcttacttgggttatgctgccgccatcatcttacggaaccgggtgcaaagcctggttctgggggcacctcgatatcagcacatcggcctggtagcgatgttcaggcagaacactggcatgtgggagtccaacgctaatgtcaagggcacccagatcggcgcctacttcggggcctccctctgctccgtggacgtggacagcaacggcagcaccgacctggtcctcatcggggccccccattactacgagcagacccgagggggccaggtgtccgtgtgccccttgcccagggggagggctcggtggcagtgtgatgctgttctctacggggagcagggccaaccctggggccgctttggggcagccctaacagtgctgggggacgtaaatggggacaagctgacggacgtggccattggggccccaggagaggaggacaaccggggtgctgtttacctgtttcacggaacctcaggatctggcatcagcccctcccatagccagcggatagcaggctccaagctctctcccaggctccagtattttggtcagtcactgagtgggggccaggacctcacaatggatggactggtagacctgactgtaggagcccaggggcacgtgctgctgctcaggtcccagccagtactgagagtcaaggcaatcatggagttcaatcccagggaagtggcaaggaatgtatttgagtgtaatgatcaggtggtgaaaggcaaggaagccggagaggtcagagtctgcctccatgtccagaagagcacacgggatcggctaagagaaggacagatccagagtgttgtgacttatgacctggctctggactccggccgcccacattcccgcgccgtcttcaatgagacaaagaacagcacacgcagacagacacaggtcttggggctgacccagacttgtgagaccctgaaactacagttgccgaattgcatcgaggacccagtgagccccattgtgctgcgcctgaacttctctctggtgggaacgccattgtctgctttcgggaacctccggccagtgctggcggaggatgctcagagactcttcacagccttgtttccctttgagaagaattgtggcaatgacaacatctgccaggatgacctcagcatcaccttcagtttcatgagcctggactgcctcgtggtgggtgggccccgggagttcaacgtgacagtgactgtgagaaatgatggtgaggactcctacaggacacaggtcaccttcttcttcccgcttgacctgtcctaccggaaggtgtccacgctccagaaccagcgctcacagcgatcctggcgcctggcctgtgagtctgcctcctccaccgaagtgtctggggccttgaagagcaccagctgcagcataaaccaccccatcttcccggaaaactcagaggtcacctttaatatcacgtttgatgtagactctaaggcttcccttggaaacaaactgctcctcaaggccaatgtgaccagtgagaacaacatgcccagaaccaacaaaaccgaattccaactggagctgccggtgaaatatgctgtctacatggtggtcaccagccatggggtctccactaaatatctcaacttcacggcctcagagaataccagtcgggtcatgcagcatcaatatcaggtcagcaacctggggcagaggagcctccccatcagcctggtgttcttggtgcccgtccggctgaaccagactgtcatatgggaccgcccccaggtcaccttctccgagaacctctcgagtacgtgccacaccaaggagcgcttgccctctcactccgactttctggctgagcttcggaaggcccccgtggtgaactgctccatcgctgtctgccagagaatccagtgtgacatcccgttctttggcatccaggaagaattcaatgctaccctcaaaggcaacctctcgtttgactggtacatcaagacctcgcataaccacctcctgatcgtgagcacagctgagatcttgtttaacgattccgtgttcaccctgctgccgggacagggggcgtttgtgaggtcccagacggagaccaaagtggagccgttcgaggtccccaaccccctgccgctcatcgtgggcagctctgtcgggggactgctgctcctggccctcatcaccgccgcgctgtacaagctcggcttcttcaagcggcaatacaaggacatgatgagtgaagggggtcccccgggggccgaaccccagtagcggctccttcccgacagagctgcctctcggtggccagcaggactctgcccagaccacacgtagcccccaggctgctggacacgtcggacagcgaagtatccccgacaggacgggcttgggcttccatttgtgtgtgtgcaagtgtgtatgtgcgtgtgtgcaagtgtctgtgtgcaagtgtgtgcacatgtgtgcgtgtgcgtgcatgtgcacttgcacgcccatgtgtgagtgtgtgcaagtatgtgagtgtgtccaagtgtgtgtgcgtgtgtccatgtgtgtgcaagtgtgtgcatgtgtgcgagtgtgtgcatgtgtgtgctcaggggcgtgtggctcacgtgtgtgactcagatgtctctggcgtgtgggtaggtgacggcagcgtagcctctccggcagaagggaactgcctgggctcccttgtgcgtgggtgaagccgctgctgggttttcctccgggagaggggacggtcaatcctgtgggtgaagacagagggaaacacagcagcttctctccactgaaagaagtgggacttcccgtcgcctgcgagcctgcggcctgctggagcctgcgcagcttggatggagactccatgagaagccgtgggtggaaccaggaacctcctccacaccagcgctgatgcccaataaagatgcccactgaggaatgatgaagcttcctttctggattcatttattatttcaatgtgactttaattttttggatggataagcttgtctatggtacaaaaatcacaaggcattcaagtgtacagtgaaaagtctccctttccagatattcaagtcacctccttaaaggtagtcaagattgtgttttgaggtttccttcagacagattccaggcgatgtgcaagtgtatgcacgtgtgcacacacaccacacatacacacacacaagcttttttacacaaatggtagcatactttatattggtctgtatcttgctttttttcaccaatatttctcagacatcggttcatattaagacataaattactttttcattcttttataccgctgcatagtattccattgtgtgagtgtaccataatgtatttaaccagtcttcttttgatatactattttcattctcttgttattgcatcaatgctgagttaataaatcaaatatatgtcatttttgcatatatgtaaggataa

SEQ ID NO: 24 - DNA, 1151 bp, Homo sapiens
acagtagccctgactacagcattcctggagcccaggctcttttccacagaggaggaaagagcaggcagcagagaccatggggcccccctcagcctctccccacagagaatgcatcccctggcaggggcttctgctcacagcctcacttctaaacttctggaacccgcccaccactgccaagctcactattgaatccatgccgctcagtgtcgcagaggggaaggaggtgcttctacttgtccacaatctgccccagcatctttttggctacagctggtacaaaggggaaagagtggatggcaacagtctaattgtaggatatgtaataggaactcaacaagctaccccaggggccgcatacagcggtcgagagacaatatacaccaatgcatccctgctgatccagaatgtcacccagaatgacataggattctacaccctacaagtcataaagtcagatcttgtgaatgaagaagcaactggacagttccatgtataccaagaaaatgccccaggccttcctgtgggggccgtcgccggcatcgtgaccggggtcctggtcggagtggcgctggtggccgcgctggtgtgtttcctgctccttgccaaaactggaagaaccagcatccagcgtgacctcaaggagcagcagccccaagcccttgcccctggccgtggtccctcccacagctctgccttctcgatgtcccctctctccactgcccaggcccccctacccaaccccaggacagcagcttccatctatgaggaattgctaaaacatgacacaaacatttactgccggatggaccacaaagcagaagtggcttcttagcttccgccaggagctgctcctgtgggttgatggagagtccccaaggcccccagccctggggatggggaaggacatgaagcctgagccagagaaccagctataagtcctgagaagacactggtgtctggggacagggagggatgggggtccctgatgaatatctggagacctcgacagcctgccctaggccctgggtgggtcaggacaaaggcctctcatcaccgcagaaagcgggggcttgcagggaaagtgaatgggcctgtggcccacctggggtcacttggaaaggatctgaataaaggggacccttcctctc

SEQ ID NO: 25 - DNA, 2977 bp, Homo sapiens
gggccgctctctgacatcagagctgctgtagagcggagaggggcaggggtgaagggccacggtggtgcaacccaccacttcctccaaggaggagctgagaggaacaggaagtgtcaggactttacgacccgcgcctccagctgaggtttctagacgtgacccagggcagactggtagcaaagcccccacgcccagccaggagcaccgccgaggactccagcacaccgagggacatgctgggcctgcgccccccactgctcgccctggtggggctgctctccctcgggtgcgtcctctctcaggagtgcacgaagttcaaggtcagcagctgccgggaatgcatcgagtcggggcccggctgcacctggtgccagaagctgaacttcacagggccgggggatcctgactccattcgctgcgacacccggccacagctgctcatgaggggctgtgcggctgacgacatcatggaccccacaagcctcgctgaaacccaggaagaccacaatgggggccagaagcagctgtccccacaaaaagtgacgctttacctgcgaccaggccaggcagcagcgttcaacgtgaccttccggcgggccaagggctaccccatcgacctgtactatctgatggacctctcctactccatgcttgatgacctcaggaatgtcaagaagctaggtggcgacctgctccgggccctcaacgagatcaccgagtccggccgcattggcttcgggtccttcgtggacaagaccgtgctgccgttcgtgaacacgcaccctgataagctgcgaaacccatgccccaacaaggagaaagagtgccagcccccgtttgccttcaggcacgtgctgaagctgaccaacaactccaaccagtttcagaccgaggtcgggaagcagctgatttccggaaacctggatgcacccgagggtgggctggacgccatgatgcaggtcgccgcctgcccggaggaaatcggctggcgcaacgtcacgcggctgctggtgtttgccactgatgacggcttccatttcgcgggcgacgggaagctgggcgccatcctgacccccaacgacggccgctgtcacctggaggacaacttgtacaagaggagcaacgaattcgactacccatcggtgggccagctggcgcacaagctggctgaaaacaacatccagcccatcttcgcggtgaccagtaggatggtgaagacctacgagaaactcaccgagatcatccccaagtcagccgtgggggagctgtctgaggactccagcaatgtggtccaactcattaagaatgcttacaataaactctcctccagggtcttcctggatcacaacgccctccccgacaccctgaaagtcacctacgactccttctgcagcaatggagtgacgcacaggaaccagcccagaggtgactgtgatggcgtgcagatcaatgtcccgatcaccttccaggtgaaggtcacggccacagagtgcatccaggagcagtcgtttgtcatccgggcgctgggcttcacggacatagtgaccgtgcaggttcttccccagtgtgagtgccggtgccgggaccagagcagagaccgcagcctctgccatggcaagggcttcttggagtgcggcatctgcaggtgtgacactggctacattgggaaaaactgtgagtgccagacacagggccggagcagccaggagctggaaggaagctgccggaaggacaacaactccatcatctgctcagggctgggggactgtgtctgcgggcagtgcctgtgccacaccagcgacgtccccggcaagctgatatacgggcagtactgcgagtgtgacaccatcaactgtgagcgctacaacggccaggtctgcggcggcccggggagggggctctgcttctgcgggaagtgccgctgccacccgggctttgagggctcagcgtgccagtgcgagaggaccactgagggctgcctgaacccgcggcgtgttgagtgtagtggtcgtggccggtgccgctgcaacgtatgcgagtgccattcaggctaccagctgcctctgtgccaggagtgccccggctgcccctcaccctgtggcaagtacatctcctgcgccgagtgcctgaagttcgaaaagggcccctttgggaagaactgcagcgcggcgtgtccgggcctgcagctgtcgaacaaccccgtgaagggcaggacctgcaaggagagggactcagagggctgctgggtggcctacacgctggagcagcaggacgggatggaccgctacctcatctatgtggatgagagccgagagtgtgtggcaggccccaacatcgccgccatcgtcgggggcaccgtggcaggcatcgtgctgatcggcattctcctgctggtcatctggaaggctctgatccacctgagcgacctccgggagtacaggcgctttgagaaggagaagctcaagtcccagtggaacaatgataatccccttttcaagagcgccaccacgacggtcatgaaccccaagtttgctgagagttaggagcacttggtgaagacaaggccgtcaggacccaccatgtctgccccatcacgcggccgagacatggcttgccacagctcttgaggatgtcaccaattaaccagaaatccagttattttccgccctcaaaatgacagccatggccggccgggtgcttctgggggctcgtcggggggacagctccactctgactggcacagtctttgcatggagacttgaggagggagggcttgaggttggtgaggttaggtgcgtgtttcctgtgcaagtcaggacatcagtctgattaaaggtggtgccaatttatttacatttaaacttgtcagggtataaaatgacatcccattaattatattgttaatcaatcacgtgtatagaaaaaaaataaaacttcaatacaggctgtccatggaaaaaaaaaaaaaaaaaaaaaa

SEQ ID NO: 26 - DNA, 2426 bp, Homo sapiens
ctcttttctaagcttgtctcttaaaacccactggacgttggcacagtgctgggatgactatggagacccaaatgtctcagaatgtatgtcccagaaacctgtggctgcttcaaccattgacagttttgctgctgctggcttctgcagacagtcaagctgctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatggggatcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaaagagtaacgttatgccatgtggtcatactctcagcttgctgagtggatgacaaaaagaggggaattgttaaaggaaaatttaaatggagactggaaaaatcctgagcaaacaaaaccacctggcccttagaaatagctttaactttgcttaaactacaaacacaagcaaaacttcacggggtcatactacatacaagcataagcaaaacttaacttggatcatttctggtaaatgcttatgttagaaataagacaaccccagccaatcacaagcagcctactaacatataattaggtgactagggactttctaagaagatacctacccccaaaaaacaattatgtaattgaaaaccaaccgattgcctttattttgcttccacattttcccaataaatacttgcctgtgacattttgccactggaacactaaacttcatgaattgcgcctcagatttttcctttaacatcttttttttttttgacagagtctcaatctgttacccaggctggagtgcagtggtgctatcttggctcactgcaaacccgcctcccaggtttaagcgattctcatgcctcagcctcccagtagctgggattagaggcatgtgccatcatacccagctaatttttgtattttttattttttttttttagtagagacagggtttcgcaatgttggccaggccgatctcgaacttctggcctctagcgatctgcccgcctcggcctcccaaagtgctgggatgaccagcatcagccccaatgtccagcctctttaacatcttctttcctatgccctctctgtggatccctactgctggtttctgccttctccatgctgagaacaaaatcacctattcactgcttatgcagtcggaagctccagaagaacaaagagcccaattaccagaaccacattaagtctccattgttttgccttgggatttgagaagagaattagagaggtgaggatctggtatttcctggactaaattccccttggggaagacgaagggatgctgcagttccaaaagagaaggactcttccagagtcatctacctgagtcccaaagctccctgtcctgaaagccacagacaatatggtcccaaatgactgactgcaccttctgtgcctcagccgttcttgacatcaagaatcttctgttccacatccacacagccaatacaattagtcaaaccactgttattaacagatgtagcaacatgagaaacgcttatgttacaggttacatgagagcaatcatgtaagtctatatgacttcagaaatgttaaaatagactaacctctaacaacaaattaaaagtgattgtttcaaggtgatgcaattattgatgacctattttatttttctataatgatcatatattacctttgtaataaaacattataaccaaaaca

SEQ ID NO: 27 - DNA, 1629 bp, Homo sapiens
acacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 28 - DNA, 2072 bp, Homo sapiens
agcgtgcgggtggcctggatcccgcgcagtggcccggcgatgtcgctcgtgctgctaagcctggccgcgctgtgcaggagcgccgtaccccgagagccgaccgttcaatgtggctctgaaactgggccatctccagagtggatgctacaacatgatctaatccccggagacttgagggacctccgagtagaacctgttacaactagtgttgcaacaggggactattcaattttgatgaatgtaagctgggtactccgggcagatgccagcatccgcttgttgaaggccaccaagatttgtgtgacgggcaaaagcaacttccagtcctacagctgtgtgaggtgcaattacacagaggccttccagactcagaccagaccctctggtggtaaatggacattttcctacatcggcttccctgtagagctgaacacagtctatttcattggggcccataatattcctaatgcaaatatgaatgaagatggcccttccatgtctgtgaatttcacctcaccaggctgcctagaccacataatgaaatataaaaaaaagtgtgtcaaggccggaagcctgtgggatccgaacatcactgcttgtaagaagaatgaggagacagtagaagtgaacttcacaaccactcccctgggaaacagatacatggctcttatccaacacagcactatcatcgggttttctcaggtgtttgagccacaccagaagaaacaaacgcgagcttcagtggtgattccagtgactggggatagtgaaggtgctacggtgcagctgactccatattttcctacttgtggcagcgactgcatccgacataaaggaacagttgtgctctgcccacaaacaggcgtccctttccctctggataacaacaaaagcaagccgggaggctggctgcctctcctcctgctgtctctgctggtggccacatgggtgctggtggcagggatctatctaatgtggaggcacgaaaggatcaagaagacttccttttctaccaccacactactgccccccattaaggttcttgtggtttacccatctgaaatatgtttccatcacacaatttgttacttcactgaatttcttcaaaaccattgcagaagtgaggtcatccttgaaaagtggcagaaaaagaaaatagcagagatgggtccagtgcagtggcttgccactcaaaagaaggcagcagacaaagtcgtcttccttctttccaatgacgtcaacagtgtgtgcgatggtacctgtggcaagagcgagggcagtcccagtgagaactctcaagacctcttcccccttgcctttaaccttttctgcagtgatctaagaagccagattcatctgcacaaatacgtggtggtctactttagagagattgatacaaaagacgattacaatgctctcagtgtctgccccaagtaccacctcatgaaggatgccactgctttctgtgcagaacttctccatgtcaagcagcaggtgtcagcaggaaaaagatcacaagcctgccacgatggctgctgctccttgtagcccacccatgagaagcaagagaccttaaaggcttcctatcccaccaattacagggaaaaaacgtgtgatgatcctgaagcttactatgcagcctacaaacagccttagtaattaaaacattttataccaataaaattttcaaatattgctaactaatgtagcattaactaacgattggaaactacatttacaacttcaaagctgttttatacatagaaatcaattacagttttaattgaaaactataaccattttgataatgcaacaataaagcatcttcagccaaacatctagtcttccatagaccatgcattgcagtgtacccagaactgtttagctaatattctatgtttaattaatgaatactaactctaagaacccctcactgattcactcaatagcatcttaagtgaaaaaccttctattacatgcaaaaaatcattgtttttaagataacaaaagtagggaataaacaagctgaacccacttttaaaaaaaaaaaaaaaaaaaaaaaaaaaaaaaaa

SEQ ID NO: 29 - DNA, 2809 bp, Homo sapiens
acgcgcgccctgcggagcccgcccaactccggcgagccgggcctgcgcctactcctcctcctcctctcccggcggcggctgcggcggaggcgccgactcggccttgcgcccgccctcaggcccgcgcgggcggcgcagcgaggccccgggcggcgggtggtggctgccaggcggctcggccgcgggcgctgcccggccccggcgagcggagggcggagcgcggcgccggagccgagggcgcgccgcggagggggtgctgggccgcgctgtgcccggccgggcggcggctgcaagaggaggccggaggcgagcgcggggccggcggtgggcgcgcagggcggctcgcagctcgcagccggggccgggccaggcgtccaggcaggtgatcggtgtggcggcggcggcggcggcggccccagactccctccggagttcttcttggggctgatgtccgcaaatatgcagaattaccggccgggtcgctcctgaagccagcgcggggagcgagcgcggcggcggccagcaccgggaacgcaccgaggaagaagcccagcccccgccctccgccccttccgtccccaccccctacccggcggcccaggaggctccccgcgctgcgggcgcgcactccctgtttctcctcctcctggctggcgctgcctgcctctccgcactcactgctcgcgccgggcgcgctccgccagctccgtgctccccgcgccaccctcctccgggccgcgctccctaagggatggtactgaatttcgccgccacaggagaccggctggagcgcccgccccgcggcctcgcctctcctccgagcagccagcgcctcgggacgcgatgaggaccttggcttgcctgctgctcctcggctgcggatacctcgcccatgttctggccgaggaagccgagatcccccgcgaggtgatcgagaggctggcccgcagtcagatccacagcatccgggacctccagcgactcctggagatagactccgtagggagtgaggattctttggacaccagcctgagagctcacggggtccatgccactaagcatgtgcccgagaagcggcccctgcccattcggaggaagagaagcatcgaggaagctgtccccgctgtctgcaagaccaggacggtcatttacgagattcctcggagtcaggtcgaccccacgtccgccaacttcctgatctggcccccgtgcgtggaggtgaaacgctgcaccggctgctgcaacacgagcagtgtcaagtgccagccctcccgcgtccaccaccgcagcgtcaaggtggccaaggtggaatacgtcaggaagaagccaaaattaaaagaagtccaggtgaggttagaggagcatttggagtgcgcctgcgcgaccacaagcctgaatccggattatcgggaagaggacacgggaaggcctagggagtcaggtaaaaaacggaaaagaaaaaggttaaaacccacctaaagcagccaaccagatgtgaggtgaggatgagccgcagccctttcctgggacatggatgtacatggcgtgttacattcctgaacctactatgtacggtgctttattgccagtgtgcggtctttgttctcctccgtgaaaaactgtgtccgagaacactcgggagaacaaagagacagtgcacatttgtttaatgtgacatcaaagcaagtattgtagcactcggtgaagcagtaagaagcttccttgtcaaaaagagagagagagaaagagagagagaaaacaaaaccacaaatgacaaaaacaaaacggactcacaaaaatatctaaactcgatgagatggagggtcgccccgtgggatggaagtgcagaggtctcagcagactggatttctgtccgggtggtcacaggtgcttttttgccgaggatgcagagcctgctttgggaacgactccagaggggtgctggtgggctctgcaggggcccgcaggaagcaggaatgtcttggaaaccgccacgcgaactttagaaaccacacctcctcgctgtagtatttaagcccatacagaaaccttcctgagagccttaagtggtttttttttttgtttttgttttgttttttttttttttgtttttttttttttttttttacaccataaagtgattattaagctttcctttttactctttggctagctttttttttttttttttttttttaattatctcttggatgacatttacaccgataacacacaggctgctgtaactgtcaggacagtgcgacggtatttttcctagcaagatgcaaactaatgagatgtattaaaataaacatggtatacctacctatgcatcatttcctaaatgtttctggctttgtgtttctcccttaccctgctttatttgttaatttaagccattttgaaagaactatgcgtcaaccaatcgtacgccgtccctgcggcacctgccccagagcccgtttgtggctgagtgacaacttgttccccgcagtgcacacctagaatgctgtgttcccacgcggcacgtgagatgcattgccgcttctgtctgtgttgttggtgtgccctggtgccgtggtggcggtcactccctctgctgccagtgtttggacagaacccaaattctttatttttggtaagatattgtgctttacctgtattaacagaaatgtgtgtgtgtggtttgtttttttgtaaaggtgaagtttgtatgtttacctaatattacctgttttgtatacctgagagcctgctatgttctttttttgttgatccaaaattaaaaaaaaaaataccaccaacaaaa

SEQ ID NO: 30 - DNA, 1716 bp, Homo sapiens
ggtctctgtgtgttctaatccctgttcattctcatttactgtctaaagttgaggagatgggatgtcccagatgatagggctcctgggatttcagacccaagaccagcaggactccagtcacctctaccccagctctccaggacacagcgctcccaactctgagtgacgtcccacctctggtccttgcagcacaaccaacgtgggaatcacaccctccagacctcccacagctccaccccagactgggcgccggccctgcctccatttcagctgtgacaacctcagagccgtgttggcccaagcatgacaaggacgtatgaaaacttccagtacttggagaataaggtgaaagtccaggggtttaaaaatgggccacttcctctccagtccctcctgcagcgtctctgctctgggccctgccatctcctgctgtccctgggcctcggcctcctgctgctggtcatcatctgtgtggttggattccaaaattccaaatttcagagggacctggtgaccctgagaacagattttagcaacttcacctcaaacactgtggcggagatccaggcactgacttcccagggcagcagcttggaagaaacgatagcatctctgaaagctgaggtggagggtttcaagcaggaacggcaggcagttcattctgaaatgctcctgcgagtccagcagctggtgcaagacctgaagaaactgacctgccaggtggctactctcaacaacaatggtgaggaagcctccactgaagggacctgctgccctgtcaactgggtggagcaccaagacagctgctactggttctctcactctgggatgtcctgggccgaggctgagaagtactgccagctgaagaacgcccacctggtggtcatcaactccagggaggagcagaattttgtccagaaatatctaggctccgcatacacctggatgggcctcagtgaccctgaaggagcctggaagtgggtggatggaacagactatgcgaccggcttccagaactggaagccaggccagccagacgactggcaggggcacgggctgggtggaggcgaggactgtgctcacttccatccagacggcaggtggaatgacgacgtctgccagaggccctaccactgggtctgcgaggctggcctgggtcagaccagccaggagagtcactgagctgcctttggtgggaccacccggccacagaaatggcggtgggaggaggactcttctcacgacctcctcgcaagaccgctctgggagagaaataagcactgggagattggaagcactgctaacattttgaatttttttctctttaattttaaaaagatggtatagtgttcttaagcttttattttttttccaacttttgaaagtcaacttcatgaaggtataatttttacataataaaaatgcactcatttaaagagtagagatgactttgacaaatatgcatgcctaggtgactaccactccgatcgcaatagataacattgccatcgcccccaccagtcccctcatgcctctgggcagtccaaccacttccctgtttccaggccagtgatctacttctttttcactatttattggccttgcctcttctagagcttctagaacttcatataagtgaaatcatacactctcgtgtatatacttcatataagtgaatatatactctcgtgagaacttaaaaaaaaaaaaaaaaaa

SEQ ID NO: 31 - DNA, 1788 bp, Homo sapiens
ggtctctgtgtgttctaatccctgttcattctcatttactgtctaaagttgaggagatgggatgtcccagatgatagggctcctgggatttcagacccaagaccagcaggactccagtcacctctaccccagctctccaggacacagcgctcccaactctgagtgacgtcccacctctggtccttgcagcacaaccaacgtgggaatcacaccctccagacctcccacagctccaccccagactgggcgccggccctgcctccatttcagctgtgacaacctcagagccgtgttggcccaagcatgacaaggacgtatgaaaacttccagtacttggagaataaggtgaaagtccaggggtttaaaaatgggccacttcctctccagtccctcctgcagcgtctctgctctgggccctgccatctcctgctgtccctgggcctcggcctcctgctgctggtcatcatctgtgtggttggattccaaaattccaaatttcagagggacctggtgaccctgagaacagattttagcaacttcacctcaaacactgtggcggagatccaggcactgacttcccagggcagcagcttggaagaaacgatagcatctctgaaagctgaggtggagggtttcaagcaggaacggcaggcaggggtatctgagctccaggaacacactacgcagaaggcacacctaggccactgtccccactgcccatctgtgtgtgtcccagttcattctgaaatgctcctgcgagtccagcagctggtgcaagacctgaagaaactgacctgccaggtggctactctcaacaacaatgcctccactgaagggacctgctgccctgtcaactgggtggagcaccaagacagctgctactggttctctcactctgggatgtcctgggccgaggctgagaagtactgccagctgaagaacgcccacctggtggtcatcaactccagggaggagcagaattttgtccagaaatatctaggctccgcatacacctggatgggcctcagtgaccctgaaggagcctggaagtgggtggatggaacagactatgcgaccggcttccagaactggaagccaggccagccagacgactggcaggggcacgggctgggtggaggcgaggactgtgctcacttccatccagacggcaggtggaatgacgacgtctgccagaggccctaccactgggtctgcgaggctggcctgggtcagaccagccaggagagtcactgagctgcctttggtgggaccacccggccacagaaatggcggtgggaggaggactcttctcacgacctcctcgcaagaccgctctgggagagaaataagcactgggagattggaagcactgctaacattttgaatttttttctctttaattttaaaaagatggtatagtgttcttaagcttttattttttttccaacttttgaaagtcaacttcatgaaggtataatttttacataataaaaatgcactcatttaaagagtagagatgactttgacaaatatgcatgcctaggtgactaccactccgatcgcaatagataacattgccatcgcccccaccagtcccctcatgcctctgggcagtccaaccacttccctgtttccaggccagtgatctacttctttttcactatttattggccttgcctcttctagagcttctagaacttcatataagtgaaatcatacactctcgtgtatatacttcatataagtgaatatatactctcgtgagaacttaaaaaaaaaaaaaaaaaa

SEQ ID NO: 32 - DNA, 3216 bp, Homo sapiens
ggcagtttcctggctgaacacgccagcccaatacttaaagagagcaactcctgactccgatagagactggatggacccacaagggtgacagcccaggcggaccgatcttcccatcccacatcctccggcgcgatgccaaaaagaggctgacggcaactgggccttctgcagagaaagacctccgcttcactgccccggctggtcccaagggtcaggaagatggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatctagaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcattaaagcatgaatggtatggaactctctccaccctatatgtagtataaagaaaagtaggtttacattcatctcattccaacttcccagttcaggagtcccaaggaaagccccagcactaacgtaaatacacaacacacacactctaccctatacaactggacattgtctgcgtggttcctttctcagccgcttctgactgctgattctcccgttcacgttgcctaataaacatccttcaagaactctgggctgctacccagaaatcattttacccttggctcaatcctctaagctaacccccttctactgagccttcagtcttgaatttctaaaaaacagaggccatggcagaataatctttgggtaacttcaaaacggggcagccaaacccatgaggcaatgtcaggaacagaaggatgaatgaggtcccaggcagagaatcatacttagcaaagttttacctgtgcgttactaattggcctctttaagagttagtttctttgggattgctatgaatgataccctgaatttggcctgcactaatttgatgtttacaggtggacacacaaggtgcaaatcaatgcgtacgtttcctgagaagtgtctaaaaacaccaaaaagggatccgtacattcaatgtttatgcaaggaaggaaagaaagaaggaagtgaagagggagaagggatggaggtcacactggtagaacgtaaccacggaaaagagcgcatcaggcctggcacggtggctcaggcctataaccccagctccctaggagaccaaggcgggagcatctcttgaggccaggagtttgagaccagcctgggcagcatagcaagacacatccctacaaaaaattagaaattggctggatgtggtggcatacgcctgtagtcctagccactcaggaggctgaggcaggaggattgcttgagcccaggagttcgaggctgcagtcagtcatgatggcaccactgcactccagcctgggcaacagagcaagatcctgtctttaaggaaaaaaagacaagatgagcataccagcagtccttgaacattatcaaaaagttcagcatattagaatcaccgggaggccttgttaaaagagttcgctgggcccatcttcagagtctctgagttgttggtctggaatagagccaaatgttttgtgtgtctaacaattcccaggtgctgttgctgctgctactattccaggaacacactttgagaaccattgtgttattgctctgcacgcccacccactctcaactcccacgaaaaaaatcaacttccagagctaagatttcggtggaagtcctggttccatatctggtgcaagatctcccctcacgaatcagttgagtcaacattctagctcaacaacatcacacgattaacattaacgaaaattattcatttgggaaactatcagccagttttcacttctgaaggggcaggagagtgttatgagaaatcacggcagttttcagcagggtccagattcagattaaataactattttctgtcatttctgtgaccaaccacatacaaacagactcatctgtgcactctccccctcccccttcaggtatatgttttctgagtaaagttgaaaagaatctcagaccagaaaatatagatatatatttaaatcttacttgagtagaactgattacgacttttgggtgttgaggggtctataagatcaaaacttttccatgataatactaagatgttatcgaccatttatctgtccttctctcaaaagtgtatggtggaattttccagaagctatgtgatacgtgatgatgtcatcactctgctgttaacatataataaatttattgctattgtttataaaagaataaatgatatttttt

SEQ ID NO: 33 - DNA, 1049 bp, Homo sapiens
aaaacaacaggaagcagcttacaaactcggtgaacaactgagggaaccaaaccagagacgcgctgaacagagagaatcaggctcaaagcaagtggaagtgggcagagattccaccaggactggtgcaaggcgcagagccagccagatttgagaagaaggcaaaaagatgctggggagcagagctgtaatgctgctgttgctgctgccctggacagctcagggcagagctgtgcctgggggcagcagccctgcctggactcagtgccagcagctttcacagaagctctgcacactggcctggagtgcacatccactagtgggacacatggatctaagagaagagggagatgaagagactacaaatgatgttccccatatccagtgtggagatggctgtgacccccaaggactcagggacaacagtcagttctgcttgcaaaggatccaccagggtctgattttttatgagaagctgctaggatcggatattttcacaggggagccttctctgctccctgatagccctgtgggccagcttcatgcctccctactgggcctcagccaactcctgcagcctgagggtcaccactgggagactcagcagattccaagcctcagtcccagccagccatggcagcgtctccttctccgcttcaaaatccttcgcagcctccaggcctttgtggctgtagccgcccgggtctttgcccatggagcagcaaccctgagtccctaaaggcagcagctcaaggatggcactcagatctccatggcccagcaaggccaagataaatctaccaccccaggcacctgtgagccaacaggttaattagtccattaattttagtgggacctgcatatgttgaaaattaccaatactgactgacatgtgatgctgacctatgataaggttgagtatttattagatgggaagggaaatttggggattatttatcctcctggggacagtttggggaggattatttattgtatttatattgaattatgtacttttttcaataaagtcttatttttgtggctaaaaaaaa

SEQ ID NO: 34 - DNA, 1486 bp, Homo sapiens
gactccgggtggcaggcgcccgggggaatcccagctgactcgctcactgccttcgaagtccggcgccccccgggagggaactgggtggccgcaccctcccggctgcggtggctgtcgccccccaccctgcagccaggactcgatggagaatccattccaatatatggccatgtggctctttggagcaatgttccatcatgttccatgctgctgacgtcacatggagcacagaaatcaatgttagcagatagccagcccatacaagatcgtattgtattgtaggaggcattgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgccagtgtggaaccactgactactggctctcattgacttccttactaagcatagcaaacagaggaagaatttgttatcagtaagaaaaagaagaactatatgtgaatcctcttctttatactgtaatttagttattgatgtataaagcaactgttatgaaataaagaaattgcaataactggcaaaaaaaaaaaaaaaaaa

SEQ ID NO: 35 - DNA, 23 bp, Artificial Sequence
  Synthetic sequence
cagatacatcccattccttccta

SEQ ID NO: 36 - DNA, 2537 bp, Homo sapiens
gtccaggattctggctcagagttgcaccactgggttttatattcacttggatctttagttgttttggcgcctactgaggtctgaagtttgaatcctgcagtcaattgggatggtggcttgtaccccaaagtgccattgcaacccttgtccttcctgaggaaagggtggcagttgccctgtggaattcctgccctgctccccgtgggtgtccaggctgacagaagttgggactgtgtctggctggccgtaggaggagtgttcagtggtgcgccgtatcccaacccgaggccacaaaatgcttccaatggcaaaggaatatgagaaaagtgcgtggccctcctgtcagctgcataaagagagactcccccatccagtgtatccaggccattgcggaaaacagggccgatgctgtgacccttgatggtggtttcatatacgaggcaggcctggccccctacaaactgcgacctgtagcggcggaagtctacgggaccgaaagacagccacgaactcactattatgccgtggctgtggtgaagaagggcggcagctttcagctgaacgaactgcaaggtctgaagtcctgccacacaggccttcgcaggaccgctggatggaatgtccctatagggacacttcgtccattcttgaattggacgggtccacctgagcccattgaggcagctgtggccaggttcttctcagccagctgtgttcccggtgcagataaaggacagttccccaacctgtgtcgcctgtgtgcggggacaggggaaaacaaatgtgccttctcctcccaggaaccgtacttcagctactctggtgccttcaagtgtctgagagacggggctggagacgtggcttttatcagagagagcacagtgtttgaggacctgtcagacgaggctgaaagggacgagtatgagttactctgcccagacaacactcggaagccagtggacaagttcaaagactgccatctggcccgggtcccttctcatgccgttgtggcacgaagtgtgaatggcaaggaggatgccatctggaatcttctccgccaggcacaggaaaagtttggaaaggacaagtcaccgaaattccagctctttggctcccctagtgggcagaaagatctgctgttcaaggactctgccattgggttttcgagggtgcccccgaggatagattctgggctgtaccttggctccggctacttcactgccatccagaacttgaggaaaagtgaggaggaagtggctgcccggcgtgcgcgggtcgtgtggtgtgcggtgggcgagcaggagctgcgcaagtgtaaccagtggagtggcttgagcgaaggcagcgtgacctgctcctcggcctccaccacagaggactgcatcgccctggtgctgaaaggagaagctgatgccatgagtttggatggaggatatgtgtacactgcaggcaaatgtggtttggtgcctgtcctggcagagaactacaaatcccaacaaagcagtgaccctgatcctaactgtgtggatagacctgtggaaggatatcttgctgtggcggtggttaggagatcagacactagccttacctggaactctgtgaaaggcaagaagtcctgccacaccgccgtggacaggactgcaggctggaatatccccatgggcctgctcttcaaccagacgggctcctgcaaatttgatgaatatttcagtcaaagctgtgcccctgggtctgacccgagatctaatctctgtgctctgtgtattggcgacgagcagggtgagaataagtgcgtgcccaacagcaacgagagatactacggctacactggggctttccggtgcctggctgagaatgctggagacgttgcatttgtgaaagatgtcactgtcttgcagaacactgatggaaataacaatgaggcatgggctaaggatttgaagctggcagactttgcgctgctgtgcctcgatggcaaacggaagcctgtgactgaggctagaagctgccatcttgccatggccccgaatcatgccgtggtgtctcggatggataaggtggaacgcctgaaacaggtgttgctccaccaacaggctaaatttgggagaaatggatctgactgcccggacaagttttgcttattccagtctgaaaccaaaaaccttctgttcaatgacaacactgagtgtctggccagactccatggcaaaacaacatatgaaaaatatttgggaccacagtatgtcgcaggcattactaatctgaaaaagtgctcaacctcccccctcctggaagcctgtgaattcctcaggaagtaaaaccgaagaagatggcccagctccccaagaaagcctcagccattcactgcccccagctcttctccccaggtgtgttggggccttggcctcccctgctgaaggtggggattgcccatccatctgcttacaattccctgctgtcgtcttagcaagaagtaaaatgagaaattttgttgatattctctccttaaaaaaaaaaaaaaaaaaaaaaaaaaaaaa

SEQ ID NO: 37 - DNA, 1616 bp, Homo sapiens
ctccctgtgttggtggaggatgtctgcagcagcatttaaattctgggagggcttggttgtcagcagcagcaggaggaggcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaatttggtggtgtcaattgcttatttgttttcccacggttgtccagcaattaataaaacataaccttttttactgcctaaaaaaaaaaaaaaaaaaaaaaaaaaaaaaaaaa

SEQ ID NO: 38 - DNA, 1717 bp, Homo sapiens
ctgatggtatctctgtttcaggagtggtgacgcctaagctatcactggacatatcaaggacttcactaaattagcaggtaccactggtcttcttgtgcttatccgggcaagaacttatcgaaatacaatagaagtttttacttagaagagattttcagggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaatacaataagttaactattttattttctaatgtgcctagttctttccctgcttaatgaaaagcttgttttttcagtgtgaataaataatcgtaagcaaca

SEQ ID NO: 39 - DNA, 1786 bp, Homo sapiens
ctgatggtatctctgtttcaggagtggtgacgcctaagctatcactggacatatcaaggacttcactaaattagcaggtaccactggtcttcttgtgcttatccgggcaagaacttatcgaaatacaatagaagtttttacttagaagagattttcagctgctgtggattggattatgccatttggaataagaatgctgttaagagcacacaagccaggttcctcaaggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaatacaataagttaactattttattttctaatgtgcctagttctttccctgcttaatgaaaagcttgttttttcagtgtgaataaataatcgtaagcaaca

SEQ ID NO: 40 - DNA, 1777 bp, Homo sapiens
ctgatggtatctctgtttcaggagtggtgacgcctaagctatcactggacatatcaaggacttcactaaattagcaggtaccactggtcttcttgtgcttatccgggcaagaacttatcgaaatacaatagaagtttttacttagaagagattttcagctgctgtggattggattatgccatttggaataagaatgctgttaagagcacacaagccagggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaatacaataagttaactattttattttctaatgtgcctagttctttccctgcttaatgaaaagcttgttttttcagtgtgaataaataatcgtaagcaaca

SEQ ID NO: 41 - DNA, 4745 bp, Homo sapiens
ttttctgcccttctttgctttggtggcttccttgtggttcctcagtggtgcctgcaacccctggttcacctccttccaggttctggctccttccagccatggctctcagagtccttctgttaacagccttgaccttatgtcatgggttcaacttggacactgaaaacgcaatgaccttccaagagaacgcaaggggcttcgggcagagcgtggtccagcttcagggatccagggtggtggttggagccccccaggagatagtggctgccaaccaaaggggcagcctctaccagtgcgactacagcacaggctcatgcgagcccatccgcctgcaggtccccgtggaggccgtgaacatgtccctgggcctgtccctggcagccaccaccagcccccctcagctgctggcctgtggtcccaccgtgcaccagacttgcagtgagaacacgtatgtgaaagggctctgcttcctgtttggatccaacctacggcagcagccccagaagttcccagaggccctccgagggtgtcctcaagaggatagtgacattgccttcttgattgatggctctggtagcatcatcccacatgactttcggcggatgaaggagtttgtctcaactgtgatggagcaattaaaaaagtccaaaaccttgttctctttgatgcagtactctgaagaattccggattcactttaccttcaaagagttccagaacaaccctaacccaagatcactggtgaagccaataacgcagctgcttgggcggacacacacggccacgggcatccgcaaagtggtacgagagctgtttaacatcaccaacggagcccgaaagaatgcctttaagatcctagttgtcatcacggatggagaaaagtttggcgatcccttgggatatgaggatgtcatccctgaggcagacagagagggagtcattcgctacgtcattggggtgggagatgccttccgcagtgagaaatcccgccaagagcttaataccatcgcatccaagccgcctcgtgatcacgtgttccaggtgaataactttgaggctctgaagaccattcagaaccagcttcgggagaagatctttgcgatcgagggtactcagacaggaagtagcagctcctttgagcatgagatgtctcaggaaggcttcagcgctgccatcacctctaatggccccttgctgagcactgtggggagctatgactgggctggtggagtctttctatatacatcaaaggagaaaagcaccttcatcaacatgaccagagtggattcagacatgaatgatgcttacttgggttatgctgccgccatcatcttacggaaccgggtgcaaagcctggttctgggggcacctcgatatcagcacatcggcctggtagcgatgttcaggcagaacactggcatgtgggagtccaacgctaatgtcaagggcacccagatcggcgcctacttcggggcctccctctgctccgtggacgtggacagcaacggcagcaccgacctggtcctcatcggggccccccattactacgagcagacccgagggggccaggtgtccgtgtgccccttgcccagggggcagagggctcggtggcagtgtgatgctgttctctacggggagcagggccaaccctggggccgctttggggcagccctaacagtgctgggggacgtaaatggggacaagctgacggacgtggccattggggccccaggagaggaggacaaccggggtgctgtttacctgtttcacggaacctcaggatctggcatcagcccctcccatagccagcggatagcaggctccaagctctctcccaggctccagtattttggtcagtcactgagtgggggccaggacctcacaatggatggactggtagacctgactgtaggagcccaggggcacgtgctgctgctcaggtcccagccagtactgagagtcaaggcaatcatggagttcaatcccagggaagtggcaaggaatgtatttgagtgtaatgatcaggtggtgaaaggcaaggaagccggagaggtcagagtctgcctccatgtccagaagagcacacgggatcggctaagagaaggacagatccagagtgttgtgacttatgacctggctctggactccggccgcccacattcccgcgccgtcttcaatgagacaaagaacagcacacgcagacagacacaggtcttggggctgacccagacttgtgagaccctgaaactacagttgccgaattgcatcgaggacccagtgagccccattgtgctgcgcctgaacttctctctggtgggaacgccattgtctgctttcgggaacctccggccagtgctggcggaggatgctcagagactcttcacagccttgtttccctttgagaagaattgtggcaatgacaacatctgccaggatgacctcagcatcaccttcagtttcatgagcctggactgcctcgtggtgggtgggccccgggagttcaacgtgacagtgactgtgagaaatgatggtgaggactcctacaggacacaggtcaccttcttcttcccgcttgacctgtcctaccggaaggtgtccacgctccagaaccagcgctcacagcgatcctggcgcctggcctgtgagtctgcctcctccaccgaagtgtctggggccttgaagagcaccagctgcagcataaaccaccccatcttcccggaaaactcagaggtcacctttaatatcacgtttgatgtagactctaaggcttcccttggaaacaaactgctcctcaaggccaatgtgaccagtgagaacaacatgcccagaaccaacaaaaccgaattccaactggagctgccggtgaaatatgctgtctacatggtggtcaccagccatggggtctccactaaatatctcaacttcacggcctcagagaataccagtcgggtcatgcagcatcaatatcaggtcagcaacctggggcagaggagcctccccatcagcctggtgttcttggtgcccgtccggctgaaccagactgtcatatgggaccgcccccaggtcaccttctccgagaacctctcgagtacgtgccacaccaaggagcgcttgccctctcactccgactttctggctgagcttcggaaggcccccgtggtgaactgctccatcgctgtctgccagagaatccagtgtgacatcccgttctttggcatccaggaagaattcaatgctaccctcaaaggcaacctctcgtttgactggtacatcaagacctcgcataaccacctcctgatcgtgagcacagctgagatcttgtttaacgattccgtgttcaccctgctgccgggacagggggcgtttgtgaggtcccagacggagaccaaagtggagccgttcgaggtccccaaccccctgccgctcatcgtgggcagctctgtcgggggactgctgctcctggccctcatcaccgccgcgctgtacaagctcggcttcttcaagcggcaatacaaggacatgatgagtgaagggggtcccccgggggccgaaccccagtagcggctccttcccgacagagctgcctctcggtggccagcaggactctgcccagaccacacgtagcccccaggctgctggacacgtcggacagcgaagtatccccgacaggacgggcttgggcttccatttgtgtgtgtgcaagtgtgtatgtgcgtgtgtgcaagtgtctgtgtgcaagtgtgtgcacatgtgtgcgtgtgcgtgcatgtgcacttgcacgcccatgtgtgagtgtgtgcaagtatgtgagtgtgtccaagtgtgtgtgcgtgtgtccatgtgtgtgcaagtgtgtgcatgtgtgcgagtgtgtgcatgtgtgtgctcaggggcgtgtggctcacgtgtgtgactcagatgtctctggcgtgtgggtaggtgacggcagcgtagcctctccggcagaagggaactgcctgggctcccttgtgcgtgggtgaagccgctgctgggttttcctccgggagaggggacggtcaatcctgtgggtgaagacagagggaaacacagcagcttctctccactgaaagaagtgggacttcccgtcgcctgcgagcctgcggcctgctggagcctgcgcagcttggatggagactccatgagaagccgtgggtggaaccaggaacctcctccacaccagcgctgatgcccaataaagatgcccactgaggaatgatgaagcttcctttctggattcatttattatttcaatgtgactttaattttttggatggataagcttgtctatggtacaaaaatcacaaggcattcaagtgtacagtgaaaagtctccctttccagatattcaagtcacctccttaaaggtagtcaagattgtgttttgaggtttccttcagacagattccaggcgatgtgcaagtgtatgcacgtgtgcacacacaccacacatacacacacacaagcttttttacacaaatggtagcatactttatattggtctgtatcttgctttttttcaccaatatttctcagacatcggttcatattaagacataaattactttttcattcttttataccgctgcatagtattccattgtgtgagtgtaccataatgtatttaaccagtcttcttttgatatactattttcattctcttgttattgcatcaatgctgagttaataaatcaaatatatgtcatttttgcatatatgtaaggataa

SEQ ID NO: 42 - DNA, 2932 bp, Homo sapiens
atccagggtgaggaaggcagcccacacttttcttggagacacatccccaaagaagtcctcacgtggctccgtttgggcagaaaccatgaattgaacgggaaaagaaatatgtcaagtatcagaaagaagagtggcatgctttgacagcaagtggactccgagtccagggcagagcctcagttagggacatgctgggcctgcgccccccactgctcgccctggtggggctgctctccctcgggtgcgtcctctctcaggagtgcacgaagttcaaggtcagcagctgccgggaatgcatcgagtcggggcccggctgcacctggtgccagaagctgaacttcacagggccgggggatcctgactccattcgctgcgacacccggccacagctgctcatgaggggctgtgcggctgacgacatcatggaccccacaagcctcgctgaaacccaggaagaccacaatgggggccagaagcagctgtccccacaaaaagtgacgctttacctgcgaccaggccaggcagcagcgttcaacgtgaccttccggcgggccaagggctaccccatcgacctgtactatctgatggacctctcctactccatgcttgatgacctcaggaatgtcaagaagctaggtggcgacctgctccgggccctcaacgagatcaccgagtccggccgcattggcttcgggtccttcgtggacaagaccgtgctgccgttcgtgaacacgcaccctgataagctgcgaaacccatgccccaacaaggagaaagagtgccagcccccgtttgccttcaggcacgtgctgaagctgaccaacaactccaaccagtttcagaccgaggtcgggaagcagctgatttccggaaacctggatgcacccgagggtgggctggacgccatgatgcaggtcgccgcctgcccggaggaaatcggctggcgcaacgtcacgcggctgctggtgtttgccactgatgacggcttccatttcgcgggcgacgggaagctgggcgccatcctgacccccaacgacggccgctgtcacctggaggacaacttgtacaagaggagcaacgaattcgactacccatcggtgggccagctggcgcacaagctggctgaaaacaacatccagcccatcttcgcggtgaccagtaggatggtgaagacctacgagaaactcaccgagatcatccccaagtcagccgtgggggagctgtctgaggactccagcaatgtggtccaactcattaagaatgcttacaataaactctcctccagggtcttcctggatcacaacgccctccccgacaccctgaaagtcacctacgactccttctgcagcaatggagtgacgcacaggaaccagcccagaggtgactgtgatggcgtgcagatcaatgtcccgatcaccttccaggtgaaggtcacggccacagagtgcatccaggagcagtcgtttgtcatccgggcgctgggcttcacggacatagtgaccgtgcaggttcttccccagtgtgagtgccggtgccgggaccagagcagagaccgcagcctctgccatggcaagggcttcttggagtgcggcatctgcaggtgtgacactggctacattgggaaaaactgtgagtgccagacacagggccggagcagccaggagctggaaggaagctgccggaaggacaacaactccatcatctgctcagggctgggggactgtgtctgcgggcagtgcctgtgccacaccagcgacgtccccggcaagctgatatacgggcagtactgcgagtgtgacaccatcaactgtgagcgctacaacggccaggtctgcggcggcccggggagggggctctgcttctgcgggaagtgccgctgccacccgggctttgagggctcagcgtgccagtgcgagaggaccactgagggctgcctgaacccgcggcgtgttgagtgtagtggtcgtggccggtgccgctgcaacgtatgcgagtgccattcaggctaccagctgcctctgtgccaggagtgccccggctgcccctcaccctgtggcaagtacatctcctgcgccgagtgcctgaagttcgaaaagggcccctttgggaagaactgcagcgcggcgtgtccgggcctgcagctgtcgaacaaccccgtgaagggcaggacctgcaaggagagggactcagagggctgctgggtggcctacacgctggagcagcaggacgggatggaccgctacctcatctatgtggatgagagccgagagtgtgtggcaggccccaacatcgccgccatcgtcgggggcaccgtggcaggcatcgtgctgatcggcattctcctgctggtcatctggaaggctctgatccacctgagcgacctccgggagtacaggcgctttgagaaggagaagctcaagtcccagtggaacaatgataatccccttttcaagagcgccaccacgacggtcatgaaccccaagtttgctgagagttaggagcacttggtgaagacaaggccgtcaggacccaccatgtctgccccatcacgcggccgagacatggcttgccacagctcttgaggatgtcaccaattaaccagaaatccagttattttccgccctcaaaatgacagccatggccggccgggtgcttctgggggctcgtcggggggacagctccactctgactggcacagtctttgcatggagacttgaggagggagggcttgaggttggtgaggttaggtgcgtgtttcctgtgcaagtcaggacatcagtctgattaaaggtggtgccaatttatttacatttaaacttgtcagggtataaaatgacatcccattaattatattgttaatcaatcacgtgtatagaaaaaaaataaaacttcaatacaggctgtccatggaaaaaaaaaaaaaaaaaaaaaa

SEQ ID NO: 43 - DNA, 2429 bp, Homo sapiens
ctcttttctaagcttgtctcttaaaacccactggacgttggcacagtgctgggatgactatggagacccaaatgtctcagaatgtatgtcccagaaacctgtggctgcttcaaccattgacagttttgctgctgctggcttctgcagacagtcaagctgcagctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatggggatcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaaagagtaacgttatgccatgtggtcatactctcagcttgctgagtggatgacaaaaagaggggaattgttaaaggaaaatttaaatggagactggaaaaatcctgagcaaacaaaaccacctggcccttagaaatagctttaactttgcttaaactacaaacacaagcaaaacttcacggggtcatactacatacaagcataagcaaaacttaacttggatcatttctggtaaatgcttatgttagaaataagacaaccccagccaatcacaagcagcctactaacatataattaggtgactagggactttctaagaagatacctacccccaaaaaacaattatgtaattgaaaaccaaccgattgcctttattttgcttccacattttcccaataaatacttgcctgtgacattttgccactggaacactaaacttcatgaattgcgcctcagatttttcctttaacatcttttttttttttgacagagtctcaatctgttacccaggctggagtgcagtggtgctatcttggctcactgcaaacccgcctcccaggtttaagcgattctcatgcctcagcctcccagtagctgggattagaggcatgtgccatcatacccagctaatttttgtattttttattttttttttttagtagagacagggtttcgcaatgttggccaggccgatctcgaacttctggcctctagcgatctgcccgcctcggcctcccaaagtgctgggatgaccagcatcagccccaatgtccagcctctttaacatcttctttcctatgccctctctgtggatccctactgctggtttctgccttctccatgctgagaacaaaatcacctattcactgcttatgcagtcggaagctccagaagaacaaagagcccaattaccagaaccacattaagtctccattgttttgccttgggatttgagaagagaattagagaggtgaggatctggtatttcctggactaaattccccttggggaagacgaagggatgctgcagttccaaaagagaaggactcttccagagtcatctacctgagtcccaaagctccctgtcctgaaagccacagacaatatggtcccaaatgactgactgcaccttctgtgcctcagccgttcttgacatcaagaatcttctgttccacatccacacagccaatacaattagtcaaaccactgttattaacagatgtagcaacatgagaaacgcttatgttacaggttacatgagagcaatcatgtaagtctatatgacttcagaaatgttaaaatagactaacctctaacaacaaattaaaagtgattgtttcaaggtgatgcaattattgatgacctattttatttttctataatgatcatatattacctttgtaataaaacattataaccaaaaca

SEQ ID NO: 44 - DNA, 1510 bp, Homo sapiens
agactccagaatttgtttgccctctagggtagaatccgccaagctttgagagaaggctgtgactgctgtgctctgggcgccagctcgctccagggagtgatgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaagaggactctgtgactctgacatgccgggggactcacagccctgagagcgactccattccgtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagccaattccactgatcctgtgaaggctgcccaatttgagccacctggacgtcaaatgattgccatcagaaagagacaacctgaagaaaccaacaatgactatgaaacagctgacggcggctacatgactctgaaccccagggcacctactgacgatgataaaaacatctacctgactcttcctcccaacgaccatgtcaacagtaataactaaagagtaacgttatgccatgtggtcacactctcagcttgctgagtggatgacaaaaagaggggaattgttaaaggaaaatttaaatggagactggaaaaattcctgagcaaacaaaaccacctggcccttagaaatagctttaactttgcttaaactacaaacacaagcaaaacttcacggggtcatactacatacaagcataagcaaaacttaacttggatgatttctggtaaatgcttatgttagaaataagacaaccccagccaatcacaagcagcctactaacatataattaggtgactagggactttctaagaagatacctacccccaaaaaacaattatgtaattgaaaacccatcgattgcctttattttgcttccacattttcccaataaatacttgcctgtgacattttgccactggaacactaaacttca

SEQ ID NO: 45 - DNA, 2333 bp, Homo sapiens
gttgggactccgggtggcaggcgcccgggggaatcccagctgactcgctcactgccttcgaagtccggcgccccccgggagggaactgggtggccgcaccctcccggctgcggtggctgtcgccccccaccctgcagccaggactcgatggagaatccattccaatatatggccatgtggctctttggagcaatgttccatcatgttccatgctgctgacgtcacatggagcacagaaatcaatgttagcagatagccagcccatacaagatcgttttcaactagtggccccactgtgtccggaattgatgggttcttggtctcactgacttcaagaatgaagccgcggaccctcgcggtgagtgttacagctcttaaggtggcgcatctggagtttgttccttctgatgttcggatgtgttcggagtttcttccttctggtgggttcgtggtctcgctggctcaggagtgaagctacagaccttcgcggaggcattgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggatgcagctaatatacccagttggcccaaagcacctaacctatagttatataatctgactctcagttcagttttactctactaatgccttcatggtattgggaaccatagatttgtgcagctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgccagtgtggaaccactgactactggctctcattgacttccttactaagcatagcaaacagaggaagaatttgttatcagtaagaaaaagaagaactatatgtgaatcctcttctttatactgtaatttagttattgatgtataaagcaactgttatgaaataaagaaattgcaataactggcatataatgtccatcagtaaatcttggtggtggtggcaataataaacttctactgataggtagaatggtgtgcaagcttgtccaatcacggattgcaggccacatgcggcccaggacaactttgaatgtggcccaacacaaattcataaactttcatacatctcgtttttagctcatcagctatcattagcggtagtgtatttaaagtgtggcccaagacaattcttcttattccaatgtggcccagggaaatcaaaagattggatgcccctggtatagaaaactaatagtgacagtgttcatatttcatgctttcccaaatacaggtattttattttcacattctttttgccatgtttatataataataaagaaaaaccctgttgatttgttggagccattgttatctgacagaaaataattgtttatattttttgcactacactgtctaaaattagcaagctctcttctaatggaactgtaagaaagatgaaatatttttgttttattataaatttatttcaccttaaaaaaaaaaaa

SEQ ID NO: 46 - DNA, 2493 bp, Homo sapiens
gggatgttgggactccgggtggcaggcgcccgggggaatcccagctgactcgctcactgccttcgaagtccggcgccccccgggagggaactgggtggccgcaccctcccggctgcggtggctgtcgccccccaccctgcagccaggactcgatggaggtacagagctcggcttctttgccttgggaggggagtggtggtggttgaaagggcgatggaattttccccgaaagcctacgcccagggcccctcccagctccagcgttaccctccggtctatcctactggccgagctgccccgccttctcatggggaaaacttagccgcaacttcaatttttggtttttcctttaatgacacttctgaggctctcctagccatcctcccgcttccggaggagcgcagatcgcaggtccctttgcccctggcgtgcgactccctactgcgctgcgctcttacggcgttccaggctgctggctagcgcaaggcgggccgggcaccccgcgctccgctgggagggtgagggacgcgcgtctggcggccccagccaagctgcgggtttctgagaagacgctgtcccgcagccctgagggctgagttctgcacccagtcaagctcaggaaggccaagaaaagaatccattccaatatatggccatgtggctctttggagcaatgttccatcatgttccatgctgctgacgtcacatggagcacagaaatcaatgttagcagatagccagcccatacaagatcgtattgtattgtaggaggcattgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgccagtgtggaaccactgactactggctctcattgacttccttactaagcatagcaaacagaggaagaatttgttatcagtaagaaaaagaagaactatatgtgaatcctcttctttatactgtaatttagttattgatgtataaagcaactgttatgaaataaagaaattgcaataactggcatataatgtccatcagtaaatcttggtggtggtggcaataataaacttctactgataggtagaatggtgtgcaagcttgtccaatcacggattgcaggccacatgcggcccaggacaactttgaatgtggcccaacacaaattcataaactttcatacatctcgtttttagctcatcagctatcattagcggtagtgtatttaaagtgtggcccaagacaattcttcttattccaatgtggcccagggaaatcaaaagattggatgcccctggtatagaaaactaatagtgacagtgttcatatttcatgctttcccaaatacaggtattttattttcacattctttttgccatgtttatataataataaagaaaaaccctgttgatttgttggagccattgttatctgacagaaaataattgtttatattttttgcactacactgtctaaaattagcaagctctcttctaatggaactgtaagaaagatgaaatatttttgttttattataaatttatttcaccttaaaaaaaaaaaa

SEQ ID NO: 47 - protein, 300 aa, Homo sapiens
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 48 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 49 - protein, 287 aa, Homo sapiens
MRIAVICFCLLGITCAIPVKQADSGSSEEKQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 50 - protein, 335 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 51 - protein, 281 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHERSLWGSWLPCKSTTALRPPCCEEAQATHV

SEQ ID NO: 52 - protein, 290 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEVLELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 53 - protein, 502 aa, Homo sapiens
MSLVLLSLAALCRSAVPREPTVQCGSETGPSPEWMLQHDLIPGDLRDLRVEPVTTSVATGDYSILMNVSWVLRADASIRLLKATKICVTGKSNFQSYSCVRCNYTEAFQTQTRPSGGKWTFSYIGFPVELNTVYFIGAHNIPNANMNEDGPSMSVNFTSPGCLDHIMKYKKKCVKAGSLWDPNITACKKNEETVEVNFTTTPLGNRYMALIQHSTIIGFSQVFEPHQKKQTRASVVIPVTGDSEGATVQLTPYFPTCGSDCIRHKGTVVLCPQTGVPFPLDNNKSKPGGWLPLLLLSLLVATWVLVAGIYLMWRHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 54 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 55 - protein, 356 aa, Homo sapiens
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 56 - protein, 375 aa, Homo sapiens
MERASCLLLLLLPLVHVSATTPEPCELDDEDFRCVCNFSEPQPDWSEAFQCVSAVEVEIHAGGLNLEPFLKRVDADADPRQYADTVKALRVRRLTVGAAQVPAQLLVGALRVLAYSRLKELTLEDLKITGTMPPLPLEATGLALSSLRLRNVSWATGRSWLAELQQWLKPGLKVLSIAQAHSPAFSCEQVRAFPALTSLDLSDNPGLGERGLMAALCPHKFPAIQNLALRNTGMETPTGVCAALAAAGVQPHSLDLSHNSLRATVNPSAPRCMWSSALNSLNLSFAGLEQVPKGLPAKLRVLDLSCNRLNRAPQPDELPEVDNLTLDGNPFLVPGTALPHEGSMNSGVVPACARSTLSVGVSGTLVLLQGARGFA

SEQ ID NO: 57 - protein, 711 aa, Homo sapiens [sequence omitted]

SEQ ID NO: 58 - protein, 666 aa, Homo sapiens
MRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 59 - protein, 198 aa, Homo sapiens
MPLGLLWLGLALLGALHAQAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 60 - protein, 1153 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGQRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 61 - protein, 1152 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 62 - protein, 769 aa, Homo sapiens
MLGLRPPLLALVGLLSLGCVLSQECTKFKVSSCRECIESGPGCTWCQKLNFTGPGDPDSIRCDTRPQLLMRGCAADDIMDPTSLAETQEDHNGGQKQLSPQKVTLYLRPGQAAAFNVTFRRAKGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVQLIKNAYNKLSSRVFLDHNALPDTLKVTYDSFCSNGVTHRNQPRGDCDGVQINVPITFQVKVTATECIQEQSFVIRALGFTDIVTVQVLPQCECRCRDQSRDRSLCHGKGFLECGICRCDTGYIGKNCECQTQGRSSQELEGSCRKDNNSIICSGLGDCVCGQCLCHTSDVPGKLIYGQYCECDTINCERYNGQVCGGPGRGLCFCGKCRCHPGFEGSACQCERTTEGCLNPRRVECSGRGRCRCNVCECHSGYQLPLCQECPGCPSPCGKYISCAECLKFEKGPFGKNCSAACPGLQLSNNPVKGRTCKERDSEGCWVAYTLEQQDGMDRYLIYVDESRECVAGPNIAAIVGGTVAGIVLIGILLLVIWKALIHLSDLREYRRFEKEKLKSQWNNDNPLFKSATTTVMNPKFAES

SEQ ID NO: 63 - protein, 355 aa, Homo sapiens
MTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 64 - protein, 376 aa, Homo sapiens
MPFGIRMLLRAHKPGSSRRSEMTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 65 - protein, 373 aa, Homo sapiens
MPFGIRMLLRAHKPGRSEMTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 66 - protein, 135 aa, Homo sapiens
MVLGTIDLCSCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 67 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 68 - protein, 323 aa, Homo sapiens
MGILSFLPVLATESDWADCKSPQPWGHMLLWTAVLFLAPVAGTPAAPPKAVLKLEPQWINVLQEDSVTLTCRGTHSPESDSIPWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANSTDPVKAAQFEPPGRQMIAIRKRQPEETNNDYETADGGYMTLNPRAPTDDDKNIYLTLPPNDHVNSNN

SEQ ID NO: 69 - DNA, 3761 bp, Homo sapiens
aaatttagattttgcaaacctgtgcattgatgagagtgctattgaaacacattaagaaagattttcaacgcaggaatgtgtcatttcctttcttcatgtaccagatgctgaaatactatgagataaagattttaggtttcaattgtaaagagagagaagtggataaatcagtgctgctttctttaggacgaaagaagtatggagcagtgggatcactttcacaatcaacaggaggacactgatagctgctccgaatctgtgaaatttgatgctcgctcaatgacagctttgcttcctccgaatcctaaaaacagcccttcccttcaagagaaactgaagtccttcaaagctgcactgattgccctttacctcctcgtgtttgcagttctcatccctctcattggaatagtggcagctcaactcctgaagtgggaaacgaagaattgctcagttagttcaactaatgcaaatgatataactcaaagtctcacgggaaaaggaaatgacagcgaagaggaaatgagatttcaagaagtctttatggaacacatgagcaacatggagaagagaatccagcatattttagacatggaagccaacctcatggacacagagcatttccaaaatttcagcatgacaactgatcaaagatttaatgacattcttctgcagctaagtaccttgttttcctcagtccagggacatgggaatgcaatagatgaaatctccaagtccttaataagtttgaataccacattgcttgatttgcagctcaacatagaaaatctgaatggcaaaatccaagagaataccttcaaacaacaagaggaaatcagtaaattagaggagcgtgtttacaatgtatcagcagaaattatggctatgaaagaagaacaagtgcatttggaacaggaaataaaaggagaagtgaaagtactgaataacatcactaatgatctcagactgaaagattgggaacattctcagaccttgagaaatatcactttaattcaaggtcctcctggacccccgggtgaaaaaggagatcgaggtcccactggagaaagtggtccacgaggatttccaggtccaataggtcctccgggtcttaaaggtgatcggggagcaattggctttcctggaagtcgaggactcccaggatatgccggaaggccaggaaattctggaccaaaaggccagaaaggggaaaaggggagtggaaacacattaactccatttacgaaagttcgactggtcggtgggagcggccctcacgaggggagggtggagatactccacagcggccagtggggtacaatttgtgacgatcgctgggaagtgcgcgttggacaggtcgtctgtaggagcttgggatacccaggtgttcaagccgtgcacaaggcagctcactttggacaaggtactggtccaatatggctgaatgaagtgttttgttttgggagagaatcatctattgaagaatgtaaaattcggcaatgggggacaagagcctgttcacattctgaagatgctggagtcacttgcactttataatgcatcatattttcattcacaactatgaaatcgctgctcaaaaatgattttattaccttgttcctgtaaaatccatttaatcaatatttaagagattaagaatattgcccaaataatattttagattacaggattaatatattgaacaccttcatgcttactattttatgtctatatttaaatcattttaacttctataggtttttaaatggaattttctaatataatgacttatatgctgaattgaacattttgaagtttatagcttccagattacaaaggccaagggtaatagaaatgcataccagtaattggctccaattcataatatgttcaccaggagattacaattttttgctcttcttgtctttgtaatctatttagttgattttaattactttctgaataacggaagggatcagaagatatcttttgtgcctagattgcaaaatctccaatccacacatattgttttaaaataagaatgttatccaactattaagatatctcaatgtgcaataacttgtgtattagatatcaatgttaatgatatgtcttggccactatggaccagggagcttatttttcttgtcatgtactgacaactgtttaattgaatcatgaagtaaattgaaagcaggacatatgagaaaactgaccatcagtatatttgtccagataattggtggatcaaaaatgccacttaacaggaagtttagtttgttatgcactttaaatggaataattagcttgttacaattctaggacatggtgtttaaaatttaaatctgattaatccattttaacaaacaatgcaaacatcttcagtgcagaaggaagagtggtttcaactgtttggagtcttttatgaagtcagtcaacatttacaaccaaagggcgggggggggggtggggggtgcgtctttagtcctaaagggacaataactctgagcatgccccaaaaaagtagtttagcaaccttttgttggtagtcaacccatccccagggccatagtgtagagtgtgaaaagctaccctgaaacccagtaattctaccctgaaagtgactgcctgcagaaagaccagcagttgatattaaagcgcaaatgaattcaacctcagccctgaaaataacagaattctgaagtttcctatgactaattcacaaaaaaagtaattgtaaactagtactattatggaattactctactgttctttctttaatagtggcaaatgaaagcataagcttaagcattttttcatattctgaagtctcaccacacataataaccaagtggtagactcacagccgtccaacttaaaaaggcaaaaccttaccttggaattggaattactgtaaacagcctactgaaaatgcatttttatcatgtaacattcttctacttgtttaacattgctgattttctctggcagcataattttgtggttaagagaatgaattctgaatgtacactttctgtctcaaaccctggctgtaatttcagctagttaataattctttgtgttcagttccactatctaggtattttcttcaaaaggtaaatacaatggtttctgaaagaatcatttgcattatcagcctgtttgggatgtctgagatcagtgcctctgggttgttaatactgtattgctgtatggtatatgtatgctgatttactacttatgcgtaagtggtatgcatgggatgtctgaaatcagtgcctatgggttgtcaatagtattaactattagtgttaactgttagtattaactattagtattattaacactaataatagtactattactattactatttttattttaaaataaaatttacctttaaaataataatagtactattgctagtactagtactattgctattactagtactattactagtactagtactatgacactgttaatagtactattaacaacccataggcacttgggatgtctgagatcagtgcctatgggttgttaatactatattgctgtatggtatatgcatgctgatttaccacttatgcatagatatatctttaataagtaatctaaaaatcctttttgtatttgagagaatctactaagttcagtccagtcaagaaaagaacctaatagcaccaatacaaattgaggacttaatttactttggaatgttgaattgcatttgttccattaaaaaaaacagaaatttgcga

SEQ ID NO: 70 - DNA, 1648 bp, Homo sapiens
cagagaaggcttaggctcccgagtcaacagggcattcaccgcctggggcgcctgagtcatcaggacactgccaggagacacagaaccctagatgccctgcagaatccttcctgttacggtccccctccctgaaacatccttcattgcaatatttccaggaaaggaagggggctggctcggaggaagagaggtggggaggtgatcagggttcacagaggagggaactgaatgacatcccaggattacataaactgtcagaggcagccgaagagttcacaagtgtgaagcctggaagccggcgggtgccgctgtgtaggaaagaagctaaagcacttccagagcctgtccggagctcagaggttcggaagacttatcgaccatggagcgcgcgtcctgcttgttgctgctgctgctgccgctggtgcacgtctctgcgaccacgccagaaccttgtgagctggacgatgaagatttccgctgcgtctgcaacttctccgaacctcagcccgactggtccgaagccttccagtgtgtgtctgcagtagaggtggagatccatgccggcggtctcaacctagagccgtttctaaagcgcgtcgatgcggacgccgacccgcggcagtatgctgacacggtcaaggctctccgcgtgcggcggctcacagtgggagccgcacaggttcctgctcagctactggtaggcgccctgcgtgtgctagcgtactcccgcctcaaggaactgacgctcgaggacctaaagataaccggcaccatgcctccgctgcctctggaagccacaggacttgcactttccagcttgcgcctacgcaacgtgtcgtgggcgacagggcgttcttggctcgccgagctgcagcagtggctcaagccaggcctcaaggtactgagcattgcccaagcacactcgcctgccttttcctgcgaacaggttcgcgccttcccggcccttaccagcctagacctgtctgacaatcctggactgggcgaacgcggactgatggcggctctctgtccccacaagttcccggccatccagaatctagcgctgcgcaacacaggaatggagacgcccacaggcgtgtgcgccgcactggcggcggcaggtgtgcagccccacagcctagacctcagccacaactcgctgcgcgccaccgtaaaccctagcgctccgagatgcatgtggtccagcgccctgaactccctcaatctgtcgttcgctgggctggaacaggtgcctaaaggactgccagccaagctcagagtgctcgatctcagctgcaacagactgaacagggcgccgcagcctgacgagctgcccgaggtggataacctgacactggacgggaatcccttcctggtccctggaactgccctcccccacgagggctcaatgaactccggcgtggtcccagcctgtgcacgttcgaccctgtcggtgggggtgtcgggaaccctggtgctgctccaaggggcccggggctttgcctaagatccaagacagaataatgaatggactcaaactgccttggcttcaggggagtcccgtcaggacgttgaggacttttcgaccaattcaaccctttgccccacctttattaaaatcttaaacaacgggtcaaaaaaaaaaaaa

SEQ ID NO: 71 - DNA, 4006 bp, Homo sapiens
gaagggcagacagagtgtccaaaagcgtgagagcacgaagtgaggagaaggtggagaagagagaagaggaagaggaagaggaagagaggaagcggagggaactgcggccaggctaaaaggggaagaagaggatcagcccaaggaggaggaagaggaaaacaagacaaacagccagtgcagaggagaggaacgtgtgtccagtgtcccgatccctgcggagctagtagctgagagctctgtgccctgggcaccttgcagccctgcacctgcctgccacttccccaccgaggccatgggcccaggagttctgctgctcctgctggtggccacagcttggcatggtcagggaatcccagtgatagagcccagtgtccctgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacaccaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctgcaggagcccacacgcatcccccggatgagttcctcttcacaccagtggtggtcgcctgcatgtccatcatggccttgctgctgctgctgctcctgctgctattgtacaagtataagcagaagcccaagtaccaggtccgctggaagatcatcgagagctatgagggcaacagttatactttcatcgaccccacgcagctgccttacaacgagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaaggctgaggccatgctgggacccagcctgagccccggccaggaccccgagggaggcgtcgactataagaacatccacctcgagaagaaatatgtccgcagggacagtggcttctccagccagggtgtggacacctatgtggagatgaggcctgtctccacttcttcaaatgactccttctctgagcaagacctggacaaggaggatggacggcccctggagctccgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggactataccaatctgccgagcagcagcagaagcggtggcagcggcagcagcagcagtgagctggaggaggagagctctagtgagcacctgacctgctgcgagcaaggggatatcgcccagcccttgctgcagcccaacaactatcagttctgctgaggagttgacgacagggagtaccactctcccctcccacaaacttcaactcctccatggatggggcgacacggggagaacatacaaactctgccttcggtcatttcactcaacagctcggcccagctctgaaacttgggaaggtgagggattcaggggaggtcagaggatcccacttcctgagcatgggccatcactgccagtcaggggctgggggctgagccctcacccccccctcccctactgttctcatggtgttggcctcgtgtttgctatgccaactagtagaaccttctttcctaatccccttatcttcatggaaatggactgactttatgcctatgaagtccccaggagctacactgatactgagaaaaccaggctctttggggctagacagactggcagagagtgagatctccctctctgagaggagcagcagatgctcacagaccacactcagctcaggccccttggagcaggatggctcctctaagaatctcacaggacctcttagtctctgccctatacgccgccttcactccacagcctcacccctcccacccccatactggtactgctgtaatgagccaagtggcagctaaaagttgggggtgttctgcccagtcccgtcattctgggctagaaggcaggggaccttggcatgtggctggccacaccaagcaggaagcacaaactcccccaagctgactcatcctaactaacagtcacgccgtgggatgtctctgtccacattaaactaacagcattaatgcagtcaaaaaaaaaaaaaaaaaa

SEQ ID NO: 72 - DNA, 6736 bp, Homo sapiens
atgggcttcttgcccaagcttctcctcctggcctcattcttcccagcaggccaggcctcatggggcgtctccagtccccaggacgtgcagggtgtgaaggggtcttgcctgcttatcccctgcatcttcagcttccctgccgacgtggaggtgcccgacggcatcacggccatctggtactacgactactcgggccagcggcaggtggtgagccactcggcggaccccaagctggtggaggcccgcttccgcggccgcaccgagttcatggggaaccccgagcacagggtgtgcaacctgctgctgaaggacctgcagcccgaggactctggttcctacaacttccgcttcgagatcagtgaggtcaaccgctggtcagatgtgaaaggcaccttggtcacagtaacagaggagcccagggtgcccaccattgcctccccggtggagcttctcgagggcacagaggtggacttcaactgctccactccctacgtatgcctgcaggagcaggtcagactgcagtggcaaggccaggaccctgctcgctctgtcaccttcaacagccagaagtttgagcccaccggcgtcggccacctggagaccctccacatggccatgtcctggcaggaccacggccggatcctgcgctgccagctctccgtggccaatcacagggctcagagcgagattcacctccaagtgaagtatgcccccaagggtgtgaagatcctcctcagcccctcggggaggaacatccttccaggtgagctggtcacactcacctgccaggtgaacagcagctaccctgcagtcagttccattaagtggctcaaggatggggtacgcctccaaaccaagactggtgtgctgcacctgccccaggcagcctggagcgatgctggcgtctacacctgccaagctgagaacggcgtgggctctttggtctcaccccccatcagcctccacatcttcatggctgaggtccaggtgagcccagcaggtcccatcctggagaaccagacagtgacactagtctgcaacacacccaatgaggcacccagtgatctccgctacagctggtacaagaaccatgtcctgctggaggatgcccactcccataccctccggctgcacttggccactagggctgatactggcttctacttctgtgaggtgcagaacgtccatggcagcgagcgctcgggccctgtcagcgtggtagtcaaccacccgcctctcactccagtcctgacagccttcctggagacccaggcgggacttgtgggcatccttcactgctctgtggtcagtgagcccctggccacactggtgctgtcacatgggggtcatatcctggcctccacctccggggacagtgatcacagcccacgcttcagtggtacctctggtcccaactccctgcgcctggagatccgagacctggaggaaactgacagtggggagtacaagtgctcagccaccaactcccttggaaatgcaacctccaccctggacttccatgccaatgccgcccgtctcctcatcagcccggcagccgaggtggtggaaggacaggcagtgacactgagctgcagaagcggcctaagccccacacctgatgcccgcttctcctggtacctgaatggagccctgcttcacgagggtcccggcagcagcctcctgctccccgcggcctccagcactgacgccggctcataccactgccgggcccgggacggccacagtgccagtggcccctcttcgccagctgttctcactgtgctctacccccctcgacaaccaacattcaccaccaggctggaccttgatgccgctggggccggggctggacggcgaggcctccttttgtgccgtgtggacagcgacccccccgccaggctgcagctgctccacaaggaccgtgttgtggccacttccctgccatcagggggtggctgcagcacctgtgggggctgttccccacgcatgaaggtcaccaaagcccccaacttgctgcgtgtggagattcacaaccctttgctggaagaggagggcttgtacctctgtgaggccagcaatgccctgggcaacgcctccacctcagccaccttcaatggccaggccactgtcctggccattgcaccatcacacacacttcaggagggcacagaagccaacttgacttgcaacgtgagccgggaagctgctggcagccctgctaacttctcctggttccgaaatggggtgctgtgggcccagggtcccctggagaccgtgacactgctgcccgtggccagaactgatgctgccctttacgcctgccgcatcctgactgaggctggtgcccagctctccactcccgtgctcctgagtgtactctatcccccggaccgtccaaagctgtcagccctcctagacatgggccagggccacatggctctgttcatctgcactgtggacagccgccccctggccttgctggccttgttccatggggagcacctcctggccaccagcctgggtccccaggtcccatcccatggtcggttccaggctaaagctgaggccaactccctgaagttagaggtccgagaactgggccttggggactctggcagctaccgctgtgaggccacaaatgttcttggatcatccaacacctcactcttcttccaggtccgaggagcctgggtccaggtgtcaccatcacctgagctccaagagggccaggctgtggtcctgagctgccaggtacacacaggagtcccagaggggacctcatatcgttggtatcgggatggccagcccctccaggagtcgacctcggccacgctccgctttgcagccataactttgacacaagctggggcctatcattgccaagcccaggccccaggctcagccaccacgagcctagctgcacccatcagcctccacgtgtcctatgccccacgccacgtcacactcactaccctgatggacacaggccctggacgactgggcctcctcctgtgccgtgtggacagtgaccctccggcccagctgcggctgctccacggggatcgccttgtggcctccaccctacaaggtgtggggggacccgaaggcagctctcccaggctgcatgtggctgtggcccccaacacactgcgtctggagatccacggggctatgctggaggatgagggtgtctatatctgtgaggcctccaacaccctgggccaggcctcggcctcagctgacttcgacgctcaagctgtgaatgtgcaggtgtggcccggggctaccgtgcgggaggggcagctggtgaacctgacctgccttgtgtggaccactcacccggcccagctcacctacacatggtaccaggatgggcagcagcgcctggatgcccactccatccccctgcccaacgtcacagtcagggatgccacctcctaccgctgcggtgtgggcccccctggtcgggcaccccgcctctccagacctatcaccttggacgtcctctacgcgccccgcaacctgcgcctgacctacctcctggagagccatggcgggcagctggccctggtactgtgcactgtggacagccgcccgcccgcccagctggccctcagccacgccggtcgcctcttggcctcctcgacagcagcctctgtccccaacaccctgcgcctggagctgcgagggccacagcccagggatgagggtttctacagctgctctgcccgcagccctctgggccaggccaacacgtccctggagctgcggctggagggtgtgcgggtgatcctggctccggaggctgccgtgcctgaaggtgcccccatcacagtgacctgtgcggaccctgctgcccacgcacccacactctatacttggtaccacaacggtcgttggctgcaggagggtccagctgcctcactctcattcctggtggccacgcgggctcatgcaggcgcctactcttgccaggcccaggatgcccagggcacccgcagctcccgtcctgctgccctgcaagtcctctatgcccctcaggacgctgtcctgtcctccttccgggactccagggccagatccatggctgtgatacagtgcactgtggacagtgagccacctgctgagctggccctatctcatgatggcaaggtgctggccacgagcagcggggtccacagcttggcatcagggacaggccatgtccaggtggcccgaaacgccctacggctgcaggtgcaagatgtgcctgcaggtgatgacacctatgtttgcacagcccaaaacttgctgggctcaatcagcaccatcgggcggttgcaggtagaaggtgcacgcgtggtggcagagcctggcctggacgtgcctgagggcgctgccctgaacctcagctgccgcctcctgggtggccctgggcctgtgggcaactccacctttgcatggttctggaatgaccggcggctgcacgcggagcctgtgcccactctcgccttcacccacgtggctcgtgctcaagctgggatgtaccactgcctggctgagctccccactggggctgctgcctctgctccagtcatgctccgtgtgctctaccctcccaagacgcccaccatgatggtcttcgtggagcctgagggtggcctccggggcatcctggattgccgagtggacagcgagccgctcgccagcctgactctccaccttggcagtcgactggtggcctccagtcagccccagggtgctcctgcagagccacacatccatgtcctggcttcccccaatgccctgagggtggacatcgaggcgctgaggcccagcgaccaaggggaatacatctgttctgcctcaaatgtcctgggctctgcctctacctccacctactttggggtcagagccctgcaccgcctgcatcagttccagcagctgctctgggtcctgggactgctggtgggcctcctgctcctgctgttgggcctgggggcctgctacacctggagaaggaggcgtgtttgtaagcagagcatgggcgagaattcggtggagatggcttttcagaaagagaccacgcagctcattgatcctgatgcagccacatgtgagacctcaacctgtgccccacccctgggctgaccagtggtgttgcctgccctccggaggagaaagtggccagaatctgtgatgactccagcctatgaatgtgaatgaggcagtgttgagtcctgcccgcctctacgaaaacagctctgtgacatctgactttttatgacctggccccaagcctcttgcccccccaaaaatgggtggtgagaggtctgcccaggagggtgttgaccctggaggacactgaagagcactgagctgatctcgctctctcttctctggatctcctcccttctctccatttctccctcaaaggaagccctgccctttcacatccttctcctcgaaagtcaccctggactttggttggattgcagcatcctgcatcctcagaggctcaccaaggcattctgtattcaacagagtatcagtcagcctgctctaacaagagaccaaatacagtgacttcaacatgatagaattttatttttctctcccacgctagtctggctgttacgatggtttatgatgttggggctcaggatccttctatcttccttttctctatccctaaaatgatgcctttgattgtgaggctcaccatggccccgctttgtccacatgccctccagccagaagaaggaagagtggaggtagaagcacacccatgcccatggtggacgcaactcagaagctgcacaggacttttccactcacttcccattggctggagtattgtcacatggctactgcaagctacaagggagactgggaaatgtagtttttattttgagtccagaggacatttggaattggacttccaaaggactcccaactgtgagctcatccctgagacttttgacattgttgggaatgccaccagcaggccatgttttgtctcagtgcccatctactgagggccagggtgtgcccctggccattctggttgtgggcttcctggaagaggtgatcactctcacactaagactgaggaaataaaaaaggtttggtgttttcctagggagagagcatgccaggcagtggagttgcctaagcagacatccttgtgccagatttggcccctgaaagaagagatgccctcattcccaccaccaccccccctacccccagggactgggtactaccttactggcccttacaagagtggagggcagacacagatgttgtcagcatccttattcctgctccagatgcatctctgttcatgactgtgtgagctcctgtccttttcctggagaccctgtgtcgggctgttaaagagaatgagttaccaagaaggaatgacgtgcccctgcgaatcagggaccaacaggagagagctcttgagtgggctagtgactccccctgcagcctggtggagatggtgtgaggagcgaagagccctctgctctaggatttgggttgaaaaacagagagagaagtggggagttgccacaggagctaacacgctgggaggcagttgggggcgggtgaactttgtgtagccgaggccgcaccctccctcattccaggctcattcattttcatgctccattgccagactcttgctgggagcccgtccagaatgtcctcccaataaaactccatcctatgacgcaaaaaaaaaaaaaaaaaa

SEQ ID NO: 73 - DNA, 2960 bp, Homo sapiens
aaatttagattttgcaaacctgtgcattgatgagagtgctattgaaacacattaagaaagattttcaacgcaggaatgtgtcatttcctttcttcatgtaccagatgctgaaatactatgagataaagattttaggtttcaattgtaaagagagagaagtggataaatcagtgctgctttctttaggacgaaagaagtatggagcagtgggatcactttcacaatcaacaggaggacactgatagctgctccgaatctgtgaaatttgatgctcgctcaatgacagctttgcttcctccgaatcctaaaaacagcccttcccttcaagagaaactgaagtccttcaaagctgcactgattgccctttacctcctcgtgtttgcagttctcatccctctcattggaatagtggcagctcaactcctgaagtgggaaacgaagaattgctcagttagttcaactaatgcaaatgatataactcaaagtctcacgggaaaaggaaatgacagcgaagaggaaatgagatttcaagaagtctttatggaacacatgagcaacatggagaagagaatccagcatattttagacatggaagccaacctcatggacacagagcatttccaaaatttcagcatgacaactgatcaaagatttaatgacattcttctgcagctaagtaccttgttttcctcagtccagggacatgggaatgcaatagatgaaatctccaagtccttaataagtttgaataccacattgcttgatttgcagctcaacatagaaaatctgaatggcaaaatccaagagaataccttcaaacaacaagaggaaatcagtaaattagaggagcgtgtttacaatgtatcagcagaaattatggctatgaaagaagaacaagtgcatttggaacaggaaataaaaggagaagtgaaagtactgaataacatcactaatgatctcagactgaaagattgggaacattctcagaccttgagaaatatcactttaattcaaggtcctcctggacccccgggtgaaaaaggagatcgaggtcccactggagaaagtggtccacgaggatttccaggtccaataggtcctccgggtcttaaaggtgatcggggagcaattggctttcctggaagtcgaggactcccaggatatgccggaaggccaggaaattctggaccaaaaggccagaaaggggaaaaggggagtggaaacacattaagaccagtacaactcactgatcatattagggcagggccctcttaagatcaggtgggttgggcgggacatcctctgctaccatctcattaaaaggcccttcacctctggacaagtcatctgcacaactgacttccaagatccttttgtgactcctccaaatgactttggttcccgtgttgtacctgacttccacatggccttctctcctggtccctggtgctgtttgggcctctgctcccatgctcatacctcttcttactccaattactccaccatcacctctctcccctatcacccccagcctggacacctctcatgcacggactggagggctgctccaaccagtcctcagttctctgccacccattgacctagagtcttgaacccaatttaatttattgggttctaggagaactgctgtgttctcaccctaacttggaagagtgatgtttcagtcaagcaaagcgattcctaccatacaatataacacttgtgtgaggctctgtcctaaatatctcaattaccaatatgtggtttggtagtatttctcgccatgctttgctcatgcgcaatgagactacaactagggtgtaaattttaagtatcccatctaaaactcatacaatgataggaaaaatccatttgtttttcatttgatttttactgaggaatcagctcaatcttcaatgaatactggtctctttccaaagcatttttgatcaaagtaaagactgagtcaagggctttttttttttctttttcttgttttaagagacagagccttgttctattgcacaggctggactacacgcattcacctagagtctagaacacaatttaatttattgggttctaggagaactgtcatgagtattgataatatgagagttctttatattcaaacattattctcaaccagagatagggatgtcatagaagaaaatccattcattcaatcattaattcacatgtccattatgtacctccatgagctggacataacagctaataagagataattgtctctggttttacagagctaattgtccctaagagatgtagacaaatgaacaagcaattacaatacatctaagctatactgggggaggaacagggctggataggtatgcagaggagataaaaaaattttaattccttagaatattttttaaaaattgattcttattttaccttctcatcttcttattttccaaattacagcatatatatatatatatatatatatatatatatatatatatatatatatattttttttttttttttttttttttttttaagttttgaagtgtagtcgagcttgggcaatttatccaacccatttaaaccaaaaataaaacttttcatgtattacctggtcatttcaaacaaaaatattttgatcatgaaaaagaataccaatattcttttgttctaaaaatctcttatgggattacatgttatatttttggtttctctctactgatcaacagactacattttcacaactcttctttcctttacgttttaacacacagacccaagattcatactattaagattctagtagaactctagatggtatgcctctgtgtatctcagcatttttattcccactcttgtataatgaacatgttaacacctacctcacagggttgttgtgaggatcaagtaagatattgtgtgtgtgaagatgctctgtgaaatcataaagtcctttaaagatgtaa

SEQ ID NO: 74 - DNA, 3572 bp, Homo sapiens
aaatttagattttgcaaacctgtgcattgatgagagtgctattgaaacacattaagaaagattttcaacgcaggaatgtgtcatttcctttcttcatgtaccagatgctgaaatactatgagataaagattttaggtttcaattgtaaagagagagaagtggataaatcagtgctgctttctttaggacgaaagaagtatggagcagtgggatcactttcacaatcaacaggaggacactgatagctgctccgaatctgtgaaatttgatgctcgctcaatgacagctttgcttcctccgaatcctaaaaacagcccttcccttcaagagaaactgaagtccttcaaagctgcactgattgccctttacctcctcgtgtttgcagttctcatccctctcattggaatagtggcagctcaactcctgaagtgggaaacgaagaattgctcagttagttcaactaatgcaaatgatataactcaaagtctcacgggaaaaggaaatgacagcgaagaggaaatgagatttcaagaagtctttatggaacacatgagcaacatggagaagagaatccagcatattttagacatggaagccaacctcatggacacagagcatttccaaaatttcagcatgacaactgatcaaagatttaatgacattcttctgcagctaagtaccttgttttcctcagtccagggacatgggaatgcaatagatgaaatctccaagtccttaataagtttgaataccacattgcttgatttgcagctcaacatagaaaatctgaatggcaaaatccaagagaataccttcaaacaacaagaggaaatcagtaaattagaggagcgtgtttacaatgtatcagcagaaattatggctatgaaagaagaacaagtgcatttggaacaggaaataaaaggagaagtgaaagtactgaataacatcactaatgatctcagactgaaagattgggaacattctcagaccttgagaaatatcactttaattcaaggtcctcctggacccccgggtgaaaaaggagatcgaggtcccactggagaaagtggtccacgaggatttccaggtccaataggtcctccgggtcttaaaggtgatcggggagcaattggctttcctggaagtcgaggactcccaggatatgccggaaggccaggaaattctggaccaaaaggccagaaaggggaaaaggggagtggaaacacattaagtactggtccaatatggctgaatgaagtgttttgttttgggagagaatcatctattgaagaatgtaaaattcggcaatgggggacaagagcctgttcacattctgaagatgctggagtcacttgcactttataatgcatcatattttcattcacaactatgaaatcgctgctcaaaaatgattttattaccttgttcctgtaaaatccatttaatcaatatttaagagattaagaatattgcccaaataatattttagattacaggattaatatattgaacaccttcatgcttactattttatgtctatatttaaatcattttaacttctataggtttttaaatggaattttctaatataatgacttatatgctgaattgaacattttgaagtttatagcttccagattacaaaggccaagggtaatagaaatgcataccagtaattggctccaattcataatatgttcaccaggagattacaattttttgctcttcttgtctttgtaatctatttagttgattttaattactttctgaataacggaagggatcagaagatatcttttgtgcctagattgcaaaatctccaatccacacatattgttttaaaataagaatgttatccaactattaagatatctcaatgtgcaataacttgtgtattagatatcaatgttaatgatatgtcttggccactatggaccagggagcttatttttcttgtcatgtactgacaactgtttaattgaatcatgaagtaaattgaaagcaggacatatgagaaaactgaccatcagtatatttgtccagataattggtggatcaaaaatgccacttaacaggaagtttagtttgttatgcactttaaatggaataattagcttgttacaattctaggacatggtgtttaaaatttaaatctgattaatccattttaacaaacaatgcaaacatcttcagtgcagaaggaagagtggtttcaactgtttggagtcttttatgaagtcagtcaacatttacaaccaaagggcgggggggggggtggggggtgcgtctttagtcctaaagggacaataactctgagcatgccccaaaaaagtagtttagcaaccttttgttggtagtcaacccatccccagggccatagtgtagagtgtgaaaagctaccctgaaacccagtaattctaccctgaaagtgactgcctgcagaaagaccagcagttgatattaaagcgcaaatgaattcaacctcagccctgaaaataacagaattctgaagtttcctatgactaattcacaaaaaaagtaattgtaaactagtactattatggaattactctactgttctttctttaatagtggcaaatgaaagcataagcttaagcattttttcatattctgaagtctcaccacacataataaccaagtggtagactcacagccgtccaacttaaaaaggcaaaaccttaccttggaattggaattactgtaaacagcctactgaaaatgcatttttatcatgtaacattcttctacttgtttaacattgctgattttctctggcagcataattttgtggttaagagaatgaattctgaatgtacactttctgtctcaaaccctggctgtaatttcagctagttaataattctttgtgttcagttccactatctaggtattttcttcaaaaggtaaatacaatggtttctgaaagaatcatttgcattatcagcctgtttgggatgtctgagatcagtgcctctgggttgttaatactgtattgctgtatggtatatgtatgctgatttactacttatgcgtaagtggtatgcatgggatgtctgaaatcagtgcctatgggttgtcaatagtattaactattagtgttaactgttagtattaactattagtattattaacactaataatagtactattactattactatttttattttaaaataaaatttacctttaaaataataatagtactattgctagtactagtactattgctattactagtactattactagtactagtactatgacactgttaatagtactattaacaacccataggcacttgggatgtctgagatcagtgcctatgggttgttaatactatattgctgtatggtatatgcatgctgatttaccacttatgcatagatatatctttaataagtaatctaaaaatcctttttgtatttgagagaatctactaagttcagtccagtcaagaaaagaacctaatagcaccaatacaaattgaggacttaatttactttggaatgttgaattgcatttgttccattaaaaaaaacagaaatttgcga

(19) United States
(12) Patent Application Publication
(10) Pub. No.: US 2013/0116166 A1
(43) Pub. Date: May 9, 2013

(54) BIOMARKERS FOR IDIOPATHIC PULMONARY FIBROSIS

(71) Applicants: Bela Desai, Saratoga, CA (US); Jeanine D. Mattson, Palo Alto, CA (US); Robert Fick, JR., Palo Alto, CA (US)

(72) Inventors: Bela Desai, Saratoga, CA (US); Jeanine D. Mattson, Palo Alto, CA (US); Robert Fick, JR., Palo Alto, CA (US)

(21) Appl. No.: 13/695,404

(22) PCT Filed: Apr. 25, 2011
(86) PCT No.: PCT/US11/33754

§ 371 Date: Oct. 30, 2012

Related U.S. Application Data
(60) Provisional application No. 61/329,780, filed on Apr. 30, 2010.

(51) Int. Cl. C12Q 1/68; G01N 33/68

(52) U.S. Cl. CPC C12Q 1/686; G01N 33/6854
USPC 514/1.1; 435/6.12; 435/7.1; 506/17

(57) ABSTRACT

Biomarkers, kits, and diagnostic and treatment methods for idiopathic pulmonary fibrosis are provided.

This application claims the benefit of U.S. provisional patent application No. 61/329,780, filed Apr. 30, 2010, which is herein incorporated by reference in its entirety.

### SEQUENCE LISTING

The instant application contains a Sequence Listing which has been submitted in ASCII format via EFS-Web and is hereby incorporated by reference in its entirety. Said ASCII copy, created on Apr. 21, 2011, is named DX20107142.txt and is 221,836 bytes in size.

### FIELD OF THE INVENTION

The present application relates to biomarkers, kits, and diagnostic and treatment methods for idiopathic pulmonary fibrosis.

### BACKGROUND OF THE INVENTION

Idiopathic Pulmonary Fibrosis (IPF) is a group of progressive interstitial lung diseases (ILD) with unknown etiology and poorly understood pathogenesis, characterized clinically by respiratory failure (the cause of death in 80% of patients) and a median survival of 3-5 years (1). IPF is the most common form of idiopathic interstitial pneumonia and is characterized by insidious onset followed by a relentless deterioration of pulmonary function and 50% mortality within 3-5 years. The primary histopathologic finding of IPF is that of typical interstitial pneumonia with temporal heterogeneity of alternating zones of interstitial fibrosis with fibroblastic foci (i.e., newer fibrosis), inflammation, honeycomb changes (i.e., older fibrosis) and normal lung architecture (i.e., no evidence of fibrosis). Additionally, IPF pathology is associated with evidence of aberrant vascular remodeling.

In the United States, IPF has an incidence of 6.8 per 100,000 and a prevalence of 14 per 100,000, based on specific symptomatic guidelines (1). Pathologically, IPF is characterized by fibroblast proliferation leading to distortion of the lung architecture and collagen deposition. The role of inflammation in the pathogenesis of IPF is inconclusive, and a dysregulated repair process is thought to contribute to the pathogenesis of the lung lesions (2). However, in other clinical settings inflammation can lead to fibrosis and it has been hypothesized that IPF is caused by an initiating injury, ensuing chronic lung inflammation, repetitive lung injury and subsequent fibrotic scarring (3). Diseased lung tissue samples from IPF patients display inflammatory infiltrates which are composed mainly of T lymphocytes and macrophages, with varying numbers of other cell types such as mast cells, neutrophils, eosinophils and B-lymphocytes depending on the stage of disease (4-7). A role for alveolar macrophages in initiating and modulating the lung inflammatory response has been reported for IPF (8). An imbalance between T helper type-1 (Th1) and T helper type-2 (Th2) cytokines has also been implicated in IPF pathogenesis (9). Soluble ST2, a serum protein expressed in Th2 cells, and the pro-inflammatory cytokines IL-1α and TNFα are reported to be increased during acute exacerbations of IPF (6).

Thus, the pathogenesis of IPF is complex and the specific cause remains unknown. Current therapeutics/treatments for IPF include corticosteroids such as prednisone, oxygen therapy, pulmonary rehabilitation, and lung transplant. However, outside of Japan, there are currently no approved medications for treating IPF and no known cure. Additionally, symptomatic treatments cannot reverse scarring that has already happened. As a result, diagnosing and treating IPF as early as possible, before a lot of scarring has taken place, is very important. Thus, diagnostic tools are needed for identifying IPF patients and initiating treatments as early as possible.

### SUMMARY OF THE INVENTION

In certain embodiments, the invention relates to a method of diagnosing idiopathic pulmonary fibrosis (IPF) in a mammalian subject, comprising determining that the level of expression of at least one nucleic acid selected from the group consisting of CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LIT (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A variant 1 (SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42) in a test sample obtained from said subject is higher relative to the level of expression of the at least one nucleic acid in a control, wherein said higher level of expression is indicative of the presence of IPF in the subject from which the test sample was obtained. In a preferred embodiment, the expression of CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A variant 1 (SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42) in a test sample obtained from the subject is determined to be expressed at a higher level relative to the level of expression of the nucleic acid expression levels in a control.

In certain embodiments, the invention relates to a method of diagnosing idiopathic pulmonary fibrosis (IPF) in a mammalian subject, comprising determining the level of expression of at least one nucleic acid selected from the group consisting of IL17RB (SEQ ID NO:28), IL 10 (SEQ ID NO:27), PDGFA (variant 1 SEQ ID NO:29), CD301/Clec10a (variants 1-2: (SEQ ID NO:30 and SEQ ID NO:31), CD25/IL-2RA (SEQ ID NO:32), IL23p19 (SEQ ID NO:33) and IL-15 (variants 1-3: SEQ ID NO:46, SEQ ID NO:45, and SEQ ID NO:34), in a test sample obtained from said subject is lower relative to the level of expression of at least one nucleic acid in a control, wherein said lower level of expression is indicative of the presence of IPF in the subject from which the sample was obtained.

In certain embodiments, the invention relates to a method of diagnosing idiopathic pulmonary fibrosis (IPF) in a mammalian subject, comprising determining the level of cell surface expression of IL17RB (SEQ ID NO:53) in PBMC's from a test sample obtained from said subject is higher relative to the level of cell surface expression of IL17RB (SEQ ID NO:53) in PBMC's from a control, wherein said higher level of cell surface expression is indicative of the presence of IPF in the subject from which the test sample was obtained.

In certain embodiments, the mammalian subject is a human patient. In certain embodiments, the test sample is a whole blood sample. In certain embodiments, expression level is determined by a gene expression profiling method. In certain embodiments, method is a PCR-based method. In certain embodiments, the method is a flow cytometry-based method.

In certain embodiments, the invention relates to a method of treating idiopathic pulmonary fibrosis (IPF) in a mammalian subject in need thereof, the method comprising the steps of:

a) determining that the level of expression of at least one nucleic acid selected from the group consisting of CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A variant 1 (SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42) in a test sample obtained from said subject is higher relative to the level of expression in a control, wherein said higher level of expression is indicative of the presence of IPF in the subject from which the sample was obtained; and

b) administering to said subject an effective amount of an IPF therapeutic agent.

In certain embodiments, the invention relates to a method of treating idiopathic pulmonary fibrosis (IPF) in a mammalian subject comprising:

a) measuring expression of at least one nucleic acid selected from the group consisting of CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A variant 1 (SEQ ID NO:18), CD32aIFCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42) in a blood sample from said subject;

b) determining that said subject exhibits at least about 2-fold higher expression of the at least one nucleic acid, or any combination thereof, compared to the expression in a normal blood sample, and

c) administering to said subject an effective amount of an IPF therapeutic agent.

In certain embodiments, the invention relates to an isolated plurality of genes selected from the group consisting of CD87/UPAR (variants 1-3: SEQ ID NO: 14, SEQ NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A variant 1 (SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42).

In certain embodiments, the invention relates to an isolated plurality of genes selected from the group consisting of IL17RB (SEQ ID NO:28), IL10 (SEQ ID NO:27), PDGFA variant 1 (SEQ ID NO:29), CD301/Clec10a (variants 1-2: SEQ ID NO:30 and SEQ ID NO:31), CD25/IL-2RA (SEQ ID NO:32), IL23p19 (SEQ ID NO:33) and IL-15 (variants 1-3: SEQ ID NO:46, SEQ ID NO:45, and SEQ ID NO:34).

In certain embodiments, the invention relates to an isolated plurality of genes comprising a first group and a second group of genes, wherein said first group comprises genes selected from the group consisting of CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A variant 1 (SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42),

and said second group comprises genes selected from the group consisting of IL17RB (SEQ ID NO:28), IL10 (SEQ ID NO:27), PDGFA variant 1 (SEQ ID NO:29), CD301/Clec10a (variants 1-2: SEQ ID NO:30 and SEQ ID NO:31), CD25/IL-2RA (SEQ ID NO:32), IL23p19 (SEQ ID NO:33) and IL-15 (variants 1-3: SEQ ID NO:46, SEQ ID NO:45, and SEQ ID NO:34).

In certain embodiments, the first group of genes is differentially expressed at a higher level in a test sample obtained from a mammalian subject relative to the level of expression in a control, wherein said higher level of expression is indicative of the presence of IPF in the subject from which the test sample was obtained, and wherein each gene in said second group is differentially expressed at a lower level in a test sample obtained from the mammalian subject relative to the level of expression in a control, wherein said lower level of expression is indicative of the presence of IPF in the subject from which the test sample was obtained.

In certain embodiments, the invention provides a kit comprising a plurality of genes selected from the group consisting of CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A variant 1 (SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITG132 (variants 1-2: SEQ ID NO:25 and SEQ ID NO:42).

In certain embodiments, the invention provides a kit comprising at least one gene selected from the group consisting of CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A variant 1 (SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42).

In certain embodiments, the invention provides a kit comprising a plurality of genes selected from the group consisting of IL17RB (SEQ ID NO:28), IL10 (SEQ ID NO:27), PDGFA variant 1 (SEQ ID NO:29), CD301/Clec10a (variants 1-2: SEQ ID NO:30 and SEQ ID NO:31), CD25/IL-2RA (SEQ ID NO:32), IL23p19 (SEQ ID NO:33) and IL-15 (variants 1-3: SEQ ID NO:46, SEQ ID NO:45, and SEQ ID NO:34).

In certain embodiments, the invention provides a kit comprising at least one gene selected from the group consisting of IL17RB (SEQ ID NO:28), IL10 (SEQ ID NO:27), PDGFA variant 1 (SEQ ID NO:29), CD301/Clec10a (variants 1-2: SEQ ID NO:30 and SEQ ID NO:31), CD25/IL-2RA (SEQ ID NO:32), IL23p19 (SEQ ID NO:33) and IL-15 (variants 1-3: SEQ ID NO:46, SEQ ID NO:45, and SEQ ID NO:34).

In certain embodiments, the invention provides a kit comprising a plurality of genes comprising a first group and a second group of genes, wherein said first group comprises genes selected from the group consisting of CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A variant 1 (SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42),

and said second group comprises genes selected from the group consisting of IL17RB (SEQ ID NO:28), IL10 (SEQ ID NO:27), PDGFA variant 1 (SEQ ID NO:29), CD301/Clec10a (variants 1-2: SEQ ID NO:30 and SEQ ID NO:31), CD25/IL-2RA (SEQ ID NO:32), IL23p19 (SEQ ID NO:33) and IL-15 (variants 1-3: SEQ ID NO:46, SEQ ID NO:45, and SEQ ID NO:34).

### BRIEF DESCRIPTION OF THE DRAWINGS

FIGS. 1A-B show expression of IL-17RB (SEQ ID NO:53) on CD14 cells in PBMC from IPF (n=18) and control (n=20) subjects. The data illustrate an increase in the percentage (p=0.008) and number (p=0.018) of IL-17RB+CD14+ in IPF subjects compared to control subjects.

FIGS. 2A-B show expression of CXCR4+ cells in PBMC from IPD (n=18) and control (n=20) subjects. The data illustrate a decrease in the CXCR4+ percent (p=0.0283) (probes detects SEQ ID NO:54 variant B or SEQ ID NO:55) and number (p=0.0476) of cells in IPF patients as compared with the control subjects.

FIGS. 3A-F show differential mRNA expression as measured by RT-qPCR in the whole blood of control (n=20) and IPF (n=18) patients. The data illustrate increased mRNA levels of CD87/UPAR (variants 1-3: SEQ ID NO: 14, SEQ ID NO:15 and SEQ ID NO:16), OPN (variants 1-3: SEQ ID NO:19 SEQ ID NO:20, and SEQ ID NO:37), LTF (variants 1-2: SEQ ID NO:13 and SEQ ID NO:36), LCN2 (SEQ ID NO:22), EMR1 (SEQ ID NO:17), CD11b/ITGAM (variants 1-2: SEQ ID NO:41 and SEQ ID NO:23), as a disease signature observed in the whole blood of IPF patients.

FIGS. 4A-E show differential mRNA expression as measured by RT-qPCR in the whole blood of control subjects (n=20) and IPF (n=18) patients. The results illustrate increased mRNA levels of CEACAM3/CD66d (SEQ ID NO:24), CCR3 (variants 1-4: SEQ ID NO:21, SEQ ID NO:38, SEQ ID NO:39 and SEQ ID NO:40), CD16a variant 1 (FCRGR3A) (SEQ ID NO:18), CD32a (FCGR2A) variants 1-2 & CD32c/FCGR2c (SEQ ID NO:43, SEQ ID NO:26, and SEQ ID NO:44), and CD18 (ITGB2) (variants 1-2: SEQ ID NO:25 and SEQ ID NO:42), as a disease signature observed in the whole blood of IPF patients.

FIGS. 5A-D show differential mRNA expression as measured by RT-qPCR in the whole blood of control subjects (n=20) and IPF (n=18) patients. The data illustrate decreased mRNA levels of IL-17RB (IL-25R) (SEQ ID NO:28), IL-10 (SEQ ID NO:27), IL-2RA (SEQ ID NO:32), PDGFA variant 1 (SEQ ID NO:29) and IL-15 (variants 1-3:SEQ ID NO:46, SEQ ID NO:45, and SEQ ID NO:34) in the whole blood of IPF patients.

FIGS. 6A-B show differential protein expression as measured by ELISA in the serum of controls (n=19) and IPF (n=11) patients. The data illustrate increased protein levels of OPN(SPP1) (SEQ ID NO:47 variant B, the probe detects all three isoforms including A, B, and C (SEQ ID NO:48, 47, and 49, respectively) and CD87 (UPAR) (SEQ ID NO:50 variant 1) (the probe detects all three 3 isoforms including isoforms 2 and 3, SEQ ID NO:51 and SEQ ID NO:52, respectively) in the sera of IPF patients.

FIGS. 7A-F show gene mRNA expression in sorted PBMC from healthy donors. The expression of mRNA of different genes was measured in unsorted, IL-17RB- and IL-17RB+ cell populations.

FIGS. 8A-D show representative FACS plots showing expression of IL-17RB (SEQ ID NO:53) in PBMC of two IPF patients and two control subjects.

### DETAILED DESCRIPTION

The present invention relates to a number of disease specific signatures for IPF. The results described herein were designed to detect blood related changes in IPF patients. The results have identified cellular phenotypic markers as well as gene expression profiles from peripheral blood of IPF patients. These markers will facilitate the diagnosis of IPF patients by using blood samples—which are easy to obtain and process. Tests utilizing any combination of the gene expression and phenotypic markers described herein will provide useful diagnostic tools for identification of IPF patients, providing an opportunity to initiate treatments and therapies to prevent further lung scarring.

Gene expression analyses identified 18 differentially expressed genes out of a pool of 195 tested genes. Of these, CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A valiant 1 (SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42), were up-regulated, while IL17RB (SEQ ID NO:28), IL10 (SEQ ID NO:27), PDGFA variant 1 (SEQ ID NO:29), CD301/Clec10a (variants 1-2: SEQ ID NO:30 and SEQ ID NO:31), CD25/IL-2RA (SEQ ID NO:32), IL23p19 (SEQ ID NO:33) and IL-15 (variants 1-3: SEQ ID NO:46, SEQ ID NO:45, and SEQ ID NO:34) were down-regulated in IPF samples. Differentially regulated genes were in the functional areas of inflammation and cell signaling. Additionally, the macrophage adhesion/activation proteins CD87/UPAR (SEQ ID NO: 50, SEQ ID NO:51, and SEQ ID NO:52, variants 1-3 respectively) and OPN (SEQ ID NO:47 variant B, along with variant A SEQ ID NO:48, and variant C SEQ ID NO:49) were analyzed by ELISA and were found to correlate with their higher gene expression level in IPF patient sera.

Purified IL-17RB+ cells from healthy human PBMC expressed monocyte/macrophage associated genes CD87/UPAR CD87/UPAR (variants 1-3: SEQ ID NO: 14, SEQ ID NO:15 and SEQ ID NO:16), CD11b (variants 1-2: SEQ ID NO:41 and SEQ ID NO:23), CD18 (variants 1-2: SEQ ID NO:25 and SEQ ID NO:42) and CD16a (variant 1 SEQ ID NO:18), indicating changes in gene expression markers in the monocyte population in IPF.

Differences in cell and molecular markers involved in monocyte/macrophage activation and migration in IPF patients were determined and identified. Thus, it is expected that a role for IL-17RB (SEQ ID NO:37) expressed on CD14+ cells in IPF is likely.

In accordance with the present invention there may be employed conventional molecular biology, microbiology, protein expression and purification, antibody, and recombinant DNA techniques within the skill of the art. Such techniques are explained fully in the literature. See, e.g. Sambrook et al. (2001) Molecular Cloning: A Laboratory Manual. 3^{rd} ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.; Nucleic Acid Hybridization, Hames & Higgins eds. (1985); Transcription And Translation, Hames & Higgins, eds. (1984); Animal Cell Culture Freshney, ed. (1986); Immobilized Cells And Enzymes, IRL Press (1986); Perbal, A Practical Guide To Molecular Cloning (1984); and Harlow and Lane. Antibodies: A Laboratory Manual (Cold Spring Harbor Laboratory Press: 1988).

### DEFINITIONS

The following definitions are provided for clarity and illustrative purposes only, and are not intended to limit the scope of the invention.

As used herein, including the appended claims, the singular forms of words such as “a,” “an,” and “the” include their corresponding plural references unless the context clearly dictates otherwise. All references cited herein are incorporated by reference to the same extent as if each individual publication, patent application, or patent, was specifically and individually indicated to be incorporated by reference.

### Peripheral Blood Mononuclear Cell

A peripheral blood mononuclear cell (PBMC) is any blood cell having a round nucleus, and includes for example: a lymphocyte, a monocyte or a macrophage. These blood cells are an important component in the immune system to fight infection and adapt to intruders. The lymphocyte population contains a mixture of T cells (CD4 and CD8 positive ˜75%), B cells and NK cells (−25% combined).

PBMC cells are often extracted from whole blood using ficoll, a hydrophilic polysaccharide that separates layers of blood, with monocytes and lymphocytes forming a buffy coat under a layer of plasma. This bully coat contains the PBMCs. PBMC's can be extracted from whole blood using a hypotonic lysis which will preferentially lyse red blood cells.

### About or Approximately

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Unless otherwise stated, the term ‘about’ means within an acceptable error range for the particular value.

### Administration

In the case of the present invention, parenteral routes of administration are also possible. Such routes include intravenous, intra-arteriole, intramuscular, intradermal, subcutaneous, intraperitoneal, transmucosal, intranasal, rectal, vaginal, or transdermal routes. If desired, inactivated therapeutic formulations may be injected, e.g., intravascular, intratumor, subcutaneous, intraperitoneal, intramuscular, etc. In a preferred embodiment, the route of administration is oral. Although there are no physical limitations to delivery of the formulation, oral delivery is preferred because of its ease and convenience, and because oral formulations readily accommodate additional mixtures, such as milk and infant formula.

### Adjuvant

As used herein, the term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, and potentially useful human adjuvants such as N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, BCG (bacille Calmette-Guerin) and Corynebacterium parvum. Preferably, the adjuvant is pharmaceutically acceptable.

Amplification

“Amplification” of DNA as used herein denotes the use of polymerase chain reaction (PCR) to increase the concentration of a particular DNA sequence within a mixture of DNA sequences. For a description of PCR see Saiki et al., Science 1988, 239:487.

“Binding composition” refers to a molecule, small molecule, macromolecule, antibody, a fragment or analogue thereof, or soluble receptor, capable of binding to a target. “Binding composition” also may refer to a complex of molecules, e.g., a non-covalent complex, to an ionized molecule, and to a covalently or non-covalently modified molecule, e.g., modified by phosphorylation, acylation, cross-linking, cyclization, or limited cleavage, which is capable of binding to a target. “Binding composition” may also refer to a molecule in combination with a stabilizer, excipient, salt, buffer, solvent, or additive, capable of binding to a target. “Binding” may be defined as an association of the binding composition with a target where the association results in reduction in the normal Brownian motion of the binding composition, in cases where the binding composition can be dissolved or suspended in solution.

“Bispecific antibody” generally refers to a covalent complex, but may refer to a stable non-covalent complex of binding fragments from two different antibodies, humanized binding fragments from two different antibodies, or peptide mimetics derived from binding fragments from two different antibodies. Each binding fragment recognizes a different target or epitope, e.g., a different receptor, e.g., an inhibiting receptor and an activating receptor. Bispecific antibodies normally exhibit specific binding to two different antigens.

Endpoints in activation or inhibition can be monitored as follows. Activation, inhibition, and response to treatment, e.g., of a cell, tissue, keratinocyte, physiological fluid, organ, and animal or human subject, can be monitored by an endpoint. The endpoint may comprise a predetermined quantity or percentage of, e.g., an indicia of inflammation, oncogenicity, or cell degranulation or secretion, such as the release of a cytokine, toxic oxygen, or a protease. The endpoint may comprise, e.g., a predetermined quantity of ion flux or transport; cell migration; cell adhesion; cell proliferation; potential for metastasis; cell differentiation; and change in phenotype, e.g., change in expression of gene relating to inflammation, apoptosis, transformation, cell cycle, or metastasis (see, e.g., Knight (2000) Ann. Clin. Lab. Sci. 30:145-158; Hood and Cheresh (2002) Nature Rev. Cancer 2:91-100; Timme, et al. (2003) Curr. Drug Targets 4:251-261; Robbins and Itzkowitz (2002) Med. Clin. North Am. 86:1467-1495; Grady and Markowitz (2002) Annu. Rev. Genomics Hum. Genet. 3:101-128; Bauer, et al. (2001) Glia 36:235-243; Stanimirovic and Satoh (2000) Brain Pathol. 10:113-126).

To examine the extent of inhibition, for example, samples or assays comprising a given, e.g., protein, gene, cell, or organism, are treated with a potential activator or inhibitor and are compared to control samples without the inhibitor. Control samples, i.e., not treated with antagonist, are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 25%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

### Carrier

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E.W. Martin.

### Coding Sequence or A Sequence Encoding an Expression Product

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

### Dosage

The dosage of a therapeutic formulation will vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. In some cases, oral administration will require a higher dose than if administered intravenously.

“Exogenous” refers to substances that are produced outside an organism, cell, or human body, depending on the context. “Endogenous” refers to substances that are produced within a cell, organism, or human body, depending on the context.

### Expression Construct

By “expression construct” is meant a nucleic acid sequence comprising a target nucleic acid sequence or sequences whose expression is desired, operatively associated with expression control sequence elements which provide for the proper transcription and translation of the target nucleic acid sequence(s) within the chosen host cells. Such sequence elements may include a promoter and a polyadenylation signal. The “expression construct” may further comprise “vector sequences.” By “vector sequences” is meant any of several nucleic acid sequences established in the art which have utility in the recombinant DNA technologies of the invention to facilitate the cloning and propagation of the expression constructs including (but not limited to) plasmids, cosmids, phage vectors, viral vectors, and yeast artificial chromosomes.

Expression constructs of the present invention may comprise vector sequences that facilitate the cloning and propagation of the expression constructs. A large number of vectors, including plasmid and fungal vectors, have been described for replication and/or expression in a variety of eukaryotic and prokaryotic host cells. Standard vectors useful in the current invention are well known in the art and include (but are not limited to) plasmids, cosmids, phage vectors, viral vectors, and yeast artificial chromosomes. The vector sequences may contain a replication origin for propagation in E. coli; the SV40 origin of replication; an ampicillin, neomycin, or puromycin resistance gene for selection in host cells; and/or genes (e.g., dihydrofolate reductase gene) that amplify the dominant selectable marker plus the gene of interest.

### Express and Expression

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “transfection” means the introduction of a foreign nucleic acid into a cell. The term “transformation” means the introduction of a “foreign” (i.e. extrinsic or extracellular) gene, DNA or RNA sequence to a cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cells genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

### Expression System

The term “expression system” means a host cell and compatible vector under suitable conditions, e.g. for the expression of a protein coded for by foreign DNA carried by the vector and introduced to the host cell.

Gene or Structural Gene

The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription.

A coding sequence is “under the control of” or “operatively associated with” expression control sequences in a cell when RNA polymerase transcribes the coding sequence into RNA, particularly mRNA, which is then trans-RNA spliced (if it contains introns) and translated into the protein encoded by the coding sequence.

The term “expression control sequence” refers to a promoter and any enhancer or suppression elements that combine to regulate the transcription of a coding sequence. In a preferred embodiment, the element is an origin of replication.

A “plurality of genes” as used herein refers to a group of identified or isolated genes whose levels of expression vary in different tissues, cells or under different conditions or biological states. The different conditions may be caused by exposure to certain agents)—whether exogenous or endogenous—which include hormones, receptor ligands, chemical compounds, etc. The expression of a plurality of genes demonstrates certain patterns. That is, each gene in the plurality is expressed differently in different conditions or with or without exposure to a certain endogenous or exogenous agents. The extent or level of differential expression of each gene may vary in the plurality and may be determined qualitatively and/or quantitatively according to this invention. A gene expression profile, as used herein, refers to a plurality of genes that are differentially expressed at different levels, which constitutes a “pattern” or a “profile.” As used herein, the term “expression profile,” “profile,” “expression pattern,” “pattern,” “gene expression profile,” and “gene expression pattern” are used interchangeably.

Gene expression profiles may be measured, according to this invention, by using nucleotide or microarrays. These arrays allow tens of thousands of genes to be surveyed at the same time.

As used herein, the term “microarray” refers to nucleotide arrays that can be used to detect biomolecules, for instance to measure gene expression. “Array,” “slide,” and “chip” are used interchangeably in this disclosure. Various kinds of arrays are made in research and manufacturing facilities worldwide, some of which are available commercially. There are, for example, two main kinds of nucleotide arrays that differ in the manner in which the nucleic acid materials are placed onto the array substrate: spotted arrays and in situ synthesized arrays. One of the most widely used oligonucleotide arrays is GeneChip™ made by Affymetrix, Inc. The oligonucleotide probes that are 20- or 25-base long are synthesized in silica on the array substrate. These arrays tend to achieve high densities (e.g., more than 40,000 genes per cm²). The spotted arrays, on the other hand, tend to have lower densities, but the probes, typically partial cDNA molecules, usually are much longer than 20- or 25-mers. A representative type of spotted cDNA array is LifeArray made by Incyte Genomics. Pre-synthesized and amplified cDNA sequences are attached to the substrate of these kinds of arrays.

In one embodiment, the nucleotide is an array (i.e., a matrix) in which each position represents a discrete binding site for a product encoded by a gene (e.g., a protein or RNA), and in which binding sites are present for products of most or almost all of the genes in the organism's genome. In one embodiment, the “binding site” (hereinafter, “site”) is a nucleic acid or nucleic acid analogue to which a particular cognate cDNA can specifically hybridize. The nucleic acid or analogue of the binding site can be, e.g., a synthetic oligomer, a full-length cDNA, a less-than full length cDNA, or a gene fragment.

Although the microarray may contain binding sites for products of all or almost all genes in the target organism's genome, such comprehensiveness is not necessarily required. Usually the microarray will have binding sites corresponding to at least about 50% of the genes in the genome, often at least about 75%, more often at least about 85%, even more often more than about 90%, and most often at least about 99%. Preferably, the microarray has binding sites for genes relevant to the action of the gene expression modulating agent of interest or in a biological pathway of interest.

The nucleic acid or analogue are attached to a “solid support,” which may be made from glass, plastic (e.g., polypropylene, nylon), polyacrylamide, nitrocellulose, or other materials. A preferred method for attaching the nucleic acids to a surface is by printing on glass plates, as is described generally by Schena et al., 1995, Quantitative monitoring of gene expression patterns with a complementary DNA microarray, Science 270:467-470. This method is especially useful for preparing microarrays of cDNA. See also DeRisi et al., 1996, Use of a cDNA microarray to analyze gene expression patterns in human cancer, Nature Genetics 14:457-460; Shalon et al., 1996, A DNA microarray system for analyzing complex DNA samples using two-color fluorescent probe hybridization, Genome Res. 6:639-645; and Schena et al., 1995, Parallel human genome analysis; microarray-based expression of 1000 genes, Proc. Natl. Acad. Sci. USA 93:10539-11286.

In a preferred embodiment, the microarray is a high-density oligonucleotide array, as described above. In a particularly preferred embodiment, the nucleotide arrays are the MG_U74 and MGU74v2 arrays from Affymetrix.

“Polymerase Chain Reaction” or “PCR” is an amplification-based assay used to measure the copy number of the gene. In such assays, the corresponding nucleic acid sequences act as a template in an amplification reaction. In a quantitative amplification, the amount of amplification product will be proportional to the amount of template in the original sample. Comparison to appropriate controls provides a measure of the copy number of the gene, corresponding to the specific probe used, according to the principle discussed above. Methods of “real-time quantitative PCR” using Taqman probes are well known in the art. Detailed protocols for real-time quantitative PCR are provided, for example, for RNA in: Gibson et al., 1996, A novel method for real time quantitative RT-PCR. Genome Res. 10:995-1001; and for DNA in: Heid et al., 1996, Real time quantitative PCR. Genome Res. 10:986-994.

A TaqMan-based assay can also be used to quantify polynucleotides. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification.

Other suitable amplification methods include, but are not limited to, ligase chain reaction (LCR) (see, Wu and Wallace, 1989, Genomics 4: 560; Landegren et al., 1988 Science 241: 1077; and Barringer et al., 1990, Gene 89: 117), transcription amplification (Kwoh et al., 1989, Proc. Natl. Acad. Sci. USA 86: 1173), self-sustained sequence replication (Guatelli et al., 1990, Proc. Nat. Acad. Sci. USA 87: 1874), dot PCR, and linker adapter PCR, etc.

The “level of mRNA” in a biological sample refers to the amount of mRNA transcribed from a given gene that is present in a cell or a biological sample. One aspect of the biological state of a biological sample (e.g. a cell or cell culture) usefully measured in the present invention is its transcriptional state. The transcriptional state of a biological sample includes the identities and abundances of the constituent RNA species, especially mRNAs, in the cell under a given set of conditions. Preferably, a substantial fraction of all constituent RNA species in the biological sample are measured, but at least a sufficient fraction is measured to characterize the action of an agent or gene modulator of interest. The level of mRNA may be quantified by methods described herein or may be simply detected, by visual detection by a human, with or without comparison to a level from a control sample or a level expected of a control sample.

A “biological sample,” as used herein refers to any sample taken from a biological subject, in vivo or in situ. A biological sample may be a sample of biological tissue, or cells or a biological fluid. Biological samples may be taken, according to this invention, from any kind of biological species, any types of tissues, and any types of cells, among other things. Cell samples may be isolated cells, primary cell cultures, or cultured cell lines according to this invention. Biological samples may be combined or pooled as needed in various embodiments. Preferred samples include whole blood. Alternatively, samples may include induced sputum, bronchoalveolar lavage (BAL) fluid, and lung biopsies.

“Modulation of gene expression,” as this term is used herein, refers to the induction or inhibition of expression of a gene. Such modulation may be assessed or measured by assays. Typically, modulation of gene expression may be caused by endogenous or exogenous factors or agents. The effect of a given compound can be measured by any means known to those skilled in the art. For example, expression levels may be measured by PCR, Northern blotting, Primer Extension, Differential Display techniques, etc.

“Induction of expression” as used herein refers to any observable or measurable increase in the levels of expression of a particular gene, either qualitatively or quantitatively. The measurement of levels of expression may be carried out according to this invention using any techniques that are capable of measuring RNA transcripts in a biological sample. Examples of these techniques include, as discussed above, PCR, TaqMan, Primer Extension, Differential display and nucleotide arrays, among other things.

“Repression of expression.” “Repression” or “inhibition” of expression, are used interchangeably according to this disclosure. It refers to any observable or measurable decrease in the levels of expression of a particular gene, either qualitatively or quantitatively. The measurement of levels of expression may be carried out using any techniques that are capable of measuring RNA transcripts in a biological sample. The examples of these techniques include, as discussed above, PCR, TaqMan, Primer Extension, Differential Display, and nucleotide arrays, among other things.”

A “gene chip” or “DNA chip” is described, for instance, in U.S. Pat. Nos. 5,412,087, 5,445,934 and 5,744,305 and is useful for screening gene expression at the mRNA level. Gene chips are commercially available.

A “kit” is one or more of containers or packages, containing at least one “plurality of genes,” as described above. In certain embodiments, any desired combination of the genes are provided on a solid support. Such kits also may contain various reagents or solutions, as well as instructions for use and labels.

A “detectable label” or a “detectable moiety” is a composition that when linked with a nucleic acid or a protein molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes, biotin, digoxigenenin or haptens. A “labeled nucleic acid or oligonucleotide probe” is one that is bound, either covalently, through a linker or a chemical bond, or noncovalently through ionic, vander Waals, electrostatic, hydrophobic interactions, or hydrogen bonds, to a label such that the presence of the nucleic acid or probe may be detected by detecting the presence of the label bound to the nucleic acid or probe.

A “nucleic acid probe” is a nucleic acid capable of binding to a target nucleic acid or complementary sequence through one or more types of chemical bond, usually through complementary base pairing usually through hydrogen bond formation. As used herein, a probe may include natural (i.e., A, G, C, or T or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences that lack complete complementarity with the probe sequence depending upon the stringency of the hybridization conditions. The probes are preferably directly labeled with isotopes, for example, chromophores, luminphores, chromogens, or indirectly labeled with biotin to which a strepavidin complex may later bind. By assaying the presence or absence of the probe, one can detect the presence or absence of a target gene of interest.

“In situ hybridization” is a methodology for determining the presence of or the copy number of a gene in a sample, for example, fluorescence in situ hybridization (FISH) (see Angerer, 1987 Meth. Enzymol 152: 649). Generally, in situ hybridization comprises the following major steps: (1) fixation of tissue or biological structure to be analyzed; (2) prehybridization treatment of the biological structure to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions.

Hybridization protocols suitable for use with the methods of the invention are described, for example, in Albertson (1984) EMBO J. 3:1227-1234; Pinkel (1988) Proc. Natl. Acad. Sci. USA 85:9138-9142; EPO Pub. No. 430:402; Methods in Molecular Biology, Vol. 33: In Situ Hybridization Protocols, Chao, ed., Humana Press, Totowa, N.J. (1994); etc.

### Heterologous

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. For example, the present invention includes chimeric DNA molecules that comprise a DNA sequence and a heterologous DNA sequence which is not part of the DNA sequence. A heterologous expression regulatory element is such an element that is operatively associated with a different gene than the one it is operatively associated with in nature. In the context of the present invention, a gene encoding a protein of interest is heterologous to the vector DNA in which it is inserted for cloning or expression, and it is heterologous to a host cell containing such a vector, in which it is expressed.

### Homologous

The term “homologous” as used in the art commonly refers to the relationship between nucleic acid molecules or proteins that possess a “common evolutionary origin,” including nucleic acid molecules or proteins within superfamilies (e.g., the immunoglobulin superfamily) and nucleic acid molecules or proteins from different species (Reeck et al., Cell 1987; 50: 667). Such nucleic acid molecules or proteins have sequence homology, as reflected by their sequence similarity, whether in terms of substantial percent similarity or the presence of specific residues or motifs at conserved positions.

### Host Cell

The term “host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells can further be used for screening or other assays that are described infra. Host cells may be cultured in vitro or one or more cells in a non-human animal (e.g., a transgenic animal or a transiently transfected animal). Suitable host cells include but are not limited to Streptomyces species and E. coli.

### Immune Response

An “immune response” refers to the development in the host of a cellular and/or antibody-mediated immune response to a composition or vaccine of interest. Such a response usually consists of the subject producing antibodies, B cells, helper T cells, suppressor T cells, and/or cytotoxic T cells directed specifically to an antigen or antigens included in the composition or vaccine of interest.

### Isolated

As used herein, the term “isolated” means that the referenced material is removed from the environment in which it is normally found. Thus, an isolated biological material can be free of cellular components, i.e., components of the cells in which the material is found or produced. Isolated nucleic acid molecules include, for example, a PCR product, an isolated mRNA, a cDNA, or a restriction fragment. Isolated nucleic acid molecules also include, for example, sequences inserted into plasmids, cosmids, artificial chromosomes, and the like. An isolated nucleic acid molecule is preferably excised from the genome in which it may be found, and more preferably is no longer joined to non-regulatory sequences, non-coding sequences, or to other genes located upstream or downstream of the nucleic acid molecule when found within the genome. An isolated protein may be associated with other proteins or nucleic acids, or both, with which it associates in the cell, or with cellular membranes if it is a membrane-associated protein.

### Mutant

As used herein, the terms “mutant” and “mutation” refer to any detectable change in genetic material (e.g., DNA) or any process, mechanism, or result of such a change. This includes gene mutations, in which the structure (e.g., DNA sequence) of a gene is altered, any gene or DNA arising from any mutation process, and any expression product (e.g., protein or enzyme) expressed by a modified gene or DNA sequence. As used herein, the term “mutating” refers to a process of creating a mutant or mutation.

### Nucleic Acid Hybridization

The term “nucleic acid hybridization” refers to anti-parallel hydrogen bonding between two single-stranded nucleic acids, in which A pairs with T (or U if an RNA nucleic acid) and C pairs with G. Nucleic acid molecules are “hybridizable” to each other when at least one strand of one nucleic acid molecule can form hydrogen bonds with the complementary bases of another nucleic acid molecule under defined stringency conditions. Stringency of hybridization is determined, e.g., by (i) the temperature at which hybridization and/or washing is performed, and (ii) the ionic strength and (iii) concentration of denaturants such as formamide of the hybridization and washing solutions, as well as other parameters. Hybridization requires that the two strands contain substantially complementary sequences. Depending on the stringency of hybridization, however, some degree of mismatches may be tolerated. Under “low stringency” conditions, a greater percentage of mismatches are tolerable (i.e., will not prevent formation of an anti-parallel hybrid). See Molecular Biology of the Cell, Alberts et al., 3^{rd} ed., New York and London: Garland Publ., 1994, Ch. 7.

Typically, hybridization of two strands at high stringency requires that the sequences exhibit a high degree of complementarity over an extended portion of their length. Examples of high stringency conditions include: hybridization to filter-bound DNA in 0.5 M NaHPO₄, 7% SDS, 1 mM EDTA at 65° C., followed by washing in 0.1×SSC/0.1% SDS at 68° C. (where 1×SSC is 0.15M NaCl, 0.15M Na citrate) or for oligonucleotide molecules washing in 6×SSC/0.5% sodium pyrophosphate at about 37° C. (for 14 nucleotide-long oligos), at about 48° C. (for about 17 nucleotide-long oligos), at about 55° C. (for 20 nucleotide-long oligos), and at about 60° C. (for 23 nucleotide-long oligos)). Accordingly, the term “high stringency hybridization” refers to a combination of solvent and temperature where two strands will pair to form a “hybrid” helix only if their nucleotide sequences are almost perfectly complementary (see Molecular Biology of the Cell, Alberts et al., 3′^{d} ed., New York and London: Garland Publ., 1994, Ch. 7).

Conditions of intermediate or moderate stringency (such as, for example, an aqueous solution of 2×SSC at 65° C.; alternatively, for example, hybridization to filter-bound DNA in 0.5 M NaHPO₄, 7% SDS, 1 mM EDTA at 65° C., and washing in 0.2×SSC/0.1% SDS at 42° C.) and low stringency (such as, for example, an aqueous solution of 2×SSC at 55° C.), require correspondingly less overall complementarity for hybridization to occur between two sequences. Specific temperature and salt conditions for any given stringency hybridization reaction depend on the concentration of the target DNA and length and base composition of the probe, and are normally determined empirically in preliminary experiments, which are routine (see Southern, J. Mol. Biol. 1975; 98: 503; Sambrook et al., Molecular Cloning: A Laboratory Manual, 2^{nd} ed., vol. 2, ch. 9.50, CSH Laboratory Press, 1989; Ausubel et al. (eds.), 1989, Current Protocols in Molecular Biology, Vol. I, Green Publishing Associates, Inc., and John Wiley & Sons, Inc., New York, at p. 2.10.3).

As used herein, the term “standard hybridization conditions” refers to hybridization conditions that allow hybridization of sequences having at least 75% sequence identity. According to a specific embodiment, hybridization conditions of higher stringency may be used to allow hybridization of only sequences having at least 80% sequence identity, at least 90% sequence identity, at least 95% sequence identity, or at least 99% sequence identity.

Nucleic acid molecules that “hybridize” to any desired nucleic acids of the present invention may be of any length. In one embodiment, such nucleic acid molecules are at least 10, at least 15, at least 20, at least 30, at least 40, at least 50, and at least 70 nucleotides in length. In another embodiment, nucleic acid molecules that hybridize are of about the same length as the particular desired nucleic acid.

### Nucleic Acid Molecule

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

### Orthologs

As used herein, the term “orthologs” refers to genes in different species that apparently evolved from a common ancestral gene by speciation. Normally, orthologs retain the same function through the course of evolution. Identification of orthologs can provide reliable prediction of gene function in newly sequenced genomes. Sequence comparison algorithms that can be used to identify orthologs include without limitation BLAST, FASTA, DNA Strider, and the GCG pileup program. Orthologs often have high sequence similarity. The present invention encompasses all orthologs of the desired protein.

### Operatively Associated

By “operatively associated with” is meant that a target nucleic acid sequence and one or more expression control sequences (e.g., promoters) are physically linked so as to permit expression of the polypeptide encoded by the target nucleic acid sequence within a host cell.

### Patient or Subject

“Patient” or “subject” refers to mammals and includes human and veterinary subjects.

### Percent Sequence Similarity or Percent Sequence Identity

The terms “percent (%) sequence similarity”, “percent (%) sequence identity”, and the like, generally refer to the degree of identity or correspondence between different nucleotide sequences of nucleic acid molecules or amino acid sequences of proteins that may or may not share a common evolutionary origin (see Reeck et al., supra). Sequence identity can be determined using any of a number of publicly available sequence comparison algorithms, such as BLAST, PASTA, DNA Strider, GCG (Genetics Computer Group, Program Manual for the GCG Package, Version 7, Madison, Wis.), etc.

To determine the percent identity between two amino acid sequences or two nucleic acid molecules, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are, or are about, of the same length. The percent identity between two sequences can be determined using techniques similar to those described below, with or without allowing gaps. In calculating percent sequence identity, typically exact matches are counted.

The determination of percent identity between two sequences can be accomplished using a mathematical algorithm. A non-limiting example of a mathematical algorithm utilized for the comparison of two sequences is the algorithm of Karlin and Altschul, Proc. Natl. Acad, Sci. USA 1990, 87:2264, modified as in Karlin and Altschul, Proc. Natl. Acad. Sci. USA 1993, 90:5873-5877. Such an algorithm is incorporated into the NBLAST and XBLAST programs of Altschul et al., J. Mol. Biol. 1990; 215: 403. BLAST nucleotide searches can be performed with the NBLAST program, score=100, wordlength=12, to obtain nucleotide sequences homologous to sequences of the invention. BLAST protein searches can be performed with the XBLAST program, score=50, wordlength=3, to obtain amino acid sequences homologous to protein sequences of the invention. To obtain gapped alignments for comparison purposes, Gapped BLAST can be utilized as described in Altschul et al., Nucleic Acids Res. 1997, 25:3389. Alternatively, PSI-Blast can be used to perform an iterated search that detects distant relationship between molecules. See Altschul et al. (1997) supra. When utilizing BLAST, Gapped BLAST, and PSI-Blast programs, the default parameters of the respective programs (e.g., XBLAST and NBLAST) can be used. See ncbi.nlm.nih.gov/BLAST/ on the WorldWideWeb. Another non-limiting example of a mathematical algorithm utilized for the comparison of sequences is the algorithm of Myers and Miller, CABIOS 1988; 4: 11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

In a preferred embodiment, the percent identity between two amino acid sequences is determined using the algorithm of Needleman and Wunsch (J. Mol. Biol. 1970, 48:444-453), which has been incorporated into the GAP program in the GCG software package (Accelrys, Burlington, Mass.; available at accehys.com on the WorldWideWeb), using either a Blossum 62 matrix or a PAM250 matrix, a gap weight of 16, 14, 12, 10, 8, 6, or 4, and a length weight of 1, 2, 3, 4, 5, or 6. In yet another preferred embodiment, the percent identity between two nucleotide sequences is determined using the GAP program in the GCG software package using a NWSgapdna.CMP matrix, a gap weight of 40, 50, 60, 70, or 80, and a length weight of 1, 2, 3, 4, 5, or 6. A particularly preferred set of parameters (and the one that can be used if the practitioner is uncertain about what parameters should be applied to determine if a molecule is a sequence identity or homology limitation of the invention) is using a Blossum 62 scoring matrix with a gap open penalty of 12, a gap extend penalty of 4, and a frameshift gap penalty of 5.

In addition to the cDNA sequences encoding various desired proteins, the present invention further provides polynucleotide molecules comprising nucleotide sequences having certain percentage sequence identities to any of the aforementioned sequences. Such sequences preferably hybridize under conditions of moderate or high stringency as described above, and may include species orthologs.

### Pharmaceutically Acceptable

When formulated in a pharmaceutical composition, a therapeutic compound can be admixed with a pharmaceutically acceptable carrier or excipient. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human.

### Pharmaceutical Compositions and Administration

While it is possible to use a composition for therapy as is, it may be preferable to administer it in a pharmaceutical formulation, e.g., in admixture with a suitable pharmaceutical excipient, diluent or carrier selected with regard to the intended route of administration and standard pharmaceutical practice. Accordingly, in one aspect, the present invention provides a pharmaceutical composition or formulation comprising at least one active composition, or a pharmaceutically acceptable derivative thereof, in association with a pharmaceutically acceptable excipient, diluent and/or carrier. The excipient, diluent and/or carrier must be “acceptable” in the sense of being compatible with the other ingredients of the formulation and not deleterious to the recipient thereof.

The therapeutic compositions can be formulated for administration in any convenient way for use in human or veterinary medicine. The invention therefore includes within its scope pharmaceutical compositions comprising a product of the present invention that is adapted for use in human or veterinary medicine, including treating food allergies and related immune disorders.

In a preferred embodiment, the pharmaceutical composition is conveniently administered as an oral formulation. Oral dosage forms are well known in the art and include tablets, caplets, gelcaps, capsules, and medical foods. Tablets, for example, can be made by well-known compression techniques using wet, dry, or fluidized bed granulation methods.

Such oral formulations may be presented for use in a conventional manner with the aid of one or more suitable excipients, diluents, and carriers. Pharmaceutically acceptable excipients assist or make possible the formation of a dosage form for a bioactive material and include diluents, binding agents, lubricants, glidants, disintegrants, coloring agents, and other ingredients. Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used. An excipient is pharmaceutically acceptable if, in addition to performing its desired function, it is non-toxic, well tolerated upon ingestion, and does not interfere with absorption of bioactive materials.

Acceptable excipients, diluents, and carriers for therapeutic use are well known in the pharmaceutical art, and are described, for example, in Remington: The Science and Practice of Pharmacy. Lippincott Williams & Wilkins (A.R. Gennaro edit. 2005). The choice of pharmaceutical excipient, diluent, and carrier can be selected with regard to the intended route of administration and standard pharmaceutical practice.

The term “therapeutically effective amount” is used herein to mean an amount or dose sufficient to modulate, e.g., increase or decrease a desired activity e.g., by about 10 percent, preferably by about 50 percent, and more preferably by about 90 percent. Preferably, a therapeutically effective amount is sufficient to cause an improvement in a clinically significant condition in the host following a therapeutic regimen involving one or more therapeutic agents. The concentration or amount of the active ingredient depends on the desired dosage and administration regimen, as discussed below. Suitable dosages may range from about 0.01 mg/kg to about 100 mg/kg of body weight per day, week, or month. The pharmaceutical compositions may also include other biologically active compounds.

A therapeutically effective amount of the desired active agent can be formulated in a pharmaceutical composition to be introduced parenterally, transmucosally, e.g., orally, nasally, or rectally, or transdermally. Preferably, administration is parenteral, e.g., via intravenous injection, and also including, but is not limited to, intra-arteriole, intramuscular, intradermal, subcutaneous, intraperitoneal, intraventricular, and intracranial administration.

In another embodiment, the active ingredient can be delivered in a vesicle, in particular a liposome (see Langer, Science, 1990; 249:1527-1533; Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.).

In yet another embodiment, the therapeutic compound(s) can be delivered in a controlled release system. For example, a polypeptide may be administered using intravenous infusion with a continuous pump, in a polymer matrix such as poly-lactic/glutamic acid (PLGA), a pellet containing a mixture of cholesterol and the active ingredient (Silastic™; Dow Corning, Midland, Mich.; see U.S. Pat. No. 5,554,601) implanted subcutaneously, an implantable osmotic pump, a transdermal patch, liposomes, or other modes of administration.

The effective amounts of compounds containing active agents include doses that partially or completely achieve the desired therapeutic, prophylactic, and/or biological effect. The actual amount effective for a particular application depends on the condition being treated and the route of administration. The effective amount for use in humans can be determined from animal models. For example, a dose for humans can be formulated to achieve circulating and/or gastrointestinal concentrations that have been found to be effective in animals.

Polynucleotide or Nucleotide Sequence

A “polynucleotide” or “nucleotide sequence” is a series of nucleotide bases (also called “nucleotides”) in a nucleic acid, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide (although only sense stands are being represented herein). This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′-and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

### Promoter

The promoter sequences may be endogenous or heterologous to the host cell to be modified, and may provide ubiquitous (i.e.+, expression occurs in the absence of an apparent external stimulus) or inducible (i.e., expression only occurs in presence of particular stimuli) expression. Promoters which may be used to control gene expression include, but are not limited to, cytomegalovirus (CMV) promoter (U.S. Pat. No. 5,385,839 and No. 5,168,062), the SV40 early promoter region (Benoist and Chambon, Nature 1981; 290:304-310), the promoter contained in the 3′ long terminal repeat of Rous sarcoma virus (Yamamoto, et al., Cell 1980; 22:787-797), the herpes thymidine kinase promoter (Wagner et al., Proc. Natl. Acad. Sci. USA 1981; 78:1441-1445), the regulatory sequences of the metallothionein gene (Brinster et al., Nature 1982; 296:39-42); prokaryotic promoters such as the alkaline phosphatase promoter, the trp-lac promoter, the bacteriophage lambda P_{L} promoter, the T7 promoter, the beta-lactamase promoter (VIIIa-Komaroff, et al., Proc. Natl. Acad. Sci. USA 1978; 75:3727-3731), or the tac promoter (DeBoer, et al., Proc. Natl. Acad. Sci. USA 1983; 80:21-25); see also “Useful proteins from recombinant bacteria” in Scientific American 1980; 242:74-94; promoter elements from yeast or other fungi such as the Gal4 promoter, the ADC (alcohol dehydrogenase) promoter, and the PGK (phosphoglycerol kinase) promoter.

### Small Molecule

The term “small molecule” refers to a compound that has a molecular weight of less than about 2000 Daltons, less than about 1000 Daltons, or less than about 500 Daltons. Small molecules, without limitation, may be, for example, nucleic acids, peptides, polypeptides, peptide nucleic acids, peptidomimetics, carbohydrates, lipids, or other organic (carbon containing) or inorganic molecules and may be synthetic or naturally occurring or optionally derivatized. Such small molecules may be a therapeutically deliverable substance or may be further derivatized to facilitate delivery or targeting.

### Substantially Homologous or Substantially Similar

In a specific embodiment, two DNA sequences are “substantially homologous” or “substantially similar” when at least about 80%, and most preferably at least about 90% or 95% of the nucleotides match over the defined length of the DNA sequences, as determined by sequence comparison algorithms, such as BLAST, FASTA, DNA Strider, etc. An example of such a sequence is an allelic or species variant of the specific genes of the invention. Sequences that are substantially homologous can be identified by comparing the sequences using standard software available in sequence data banks, or in a Southern hybridization experiment under, for example, stringent conditions as defined for that particular system.

Similarly, in a particular embodiment, two amino acid sequences are “substantially homologous” or “substantially similar” when greater than 80% of the amino acids are identical, or greater than about 90% are similar. Preferably, the amino acids are functionally identical. Preferably, the similar or homologous sequences are identified by alignment using, for example, the GCG (Genetics Computer Group, Program Manual for the GCG Package, Version 10, Madison, Wis.) pileup program, or any of the programs described above (BLAST, FASTA, etc.).

### Substantially Identical

By “substantially identical” is meant a polypeptide or nucleic acid molecule exhibiting at least 80%, more preferably at least 90%, and most preferably at least 95% identity in comparison to a reference amino acid or nucleic acid sequence. For polypeptides, the length of sequence comparison will generally be at least 20 amino acids, preferably at least 30 amino acids, more preferably at least 40 amino acids, and most preferably at least 50 amino acids. For nucleic acid molecules, the length of sequence comparison will generally be at least 60 nucleotides, preferably at least 90 nucleotides, and more preferably at least 120 nucleotides.

The degree of sequence identity between any two nucleic acid molecules or two polypeptides may be determined by sequence comparison and alignment algorithms known in the art, including but not limited to BLAST, FASTA, DNA Strider, and the GCG Package (Madison, Wis.) pileup program (see, for example, Gribskov and Devereux Sequence Analysis Primer (Stockton Press: 1991) and references cited therein). The percent similarity between two nucleotide sequences may be determined, for example, using the Smith-Waterman algorithm as implemented in the BESTFIT software program using default parameters.

### Therapeutically Effective Amount

A “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

### Transfection

By “transfection” is meant the process of introducing one or more of the expression constructs of the invention into a host cell by any of the methods well established in the art, including (but not limited to) microinjection, electroporation, liposome-mediated transfection, calcium phosphate-mediated transfection, or virus-mediated transfection.

### Treating or Treatment

“Treating” or “treatment” of a state, disorder or condition includes:

(1) preventing or delaying the appearance of clinical or sub-clinical symptoms of the state, disorder or condition developing in a mammal that may be afflicted with or predisposed to the state, disorder or condition but does not yet experience or display clinical or subclinical symptoms of the state, disorder or condition; or

(2) inhibiting the state, disorder or condition, i.e., arresting, reducing or delaying the development of the disease or a relapse thereof (in case of maintenance treatment) or at least one clinical or sub-clinical symptom thereof; or

(3) relieving the disease, i.e., causing regression of the state, disorder or condition or at least one of its clinical or sub-clinical symptoms.

The benefit to a subject to be treated is either statistically significant or at least perceptible to the patient or to the physician.

### Vaccine

As used herein, the term “vaccine” refers to a composition comprising a cell or a cellular antigen, and optionally other pharmaceutically acceptable carriers, administered to stimulate an immune response in an animal, preferably a mammal, most preferably a human, specifically against the antigen and preferably to engender immunological memory that leads to mounting of a protective immune response should the subject encounter that antigen at some future time. Vaccines often comprise an adjuvant.

### Variant

The term “variant” may also be used to indicate a modified or altered gene, DNA sequence, enzyme, cell, etc., i.e., any kind of mutant.

### Vector, Cloning Vector and Expression Vector

The terms “vector”, “cloning vector” and “expression vector” refer to the vehicle by which DNA can be introduced into a host cell, resulting in expression of the introduced sequence. In one embodiment, vectors comprise a promoter and one or more control elements (e.g., enhancer elements) that are heterologous to the introduced DNA but are recognized and used by the host cell. In another embodiment, the sequence that is introduced into the vector retains its natural promoter that may be recognized and expressed by the host cell (Bormann et al., J. Bacteriol. 1996; 178:1216-1218).

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”. A common type of vector is a “plasmid”, which generally is a self-contained molecule of double-stranded DNA, usually of bacterial origin, that can readily accept additional (foreign) DNA and which can readily be introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. Recombinant cloning vectors will often include one or more replication systems for cloning or expression, one or more markers for selection in the host, e.g. antibiotic resistance, and one or more expression cassettes. Vector constructs may be produced using conventional molecular biology and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); F. M. Ausubel et al. (eds.), Current Protocols in Molecular Biology, John Wiley & Sons, Inc. (1994).

The abbreviations in the specification correspond to units of measure, techniques, properties or compounds as follows: “min” means minutes, “h” means hour(s), “μl” or “μL” means microliter(s), “ml” or “mL” means milliliter(s), “m114” means millimolar, “M” means molar, “mmole” means millimole(s), “kb” means kilobase, “bp” means base pair(s), and “IU” means International Units. “Polymerase chain reaction” is abbreviated PCR; “Reverse transcriptase polymerase chain reaction” is abbreviated RT-PCR; “DNA binding domain” is abbreviated DBD; “Untranslated region” is abbreviated UTR; “Sodium dodecyl sulfate” is abbreviated SDS; and “High Pressure Liquid Chromatography” is abbreviated HPLC.

### General Methods

Whole blood samples in the amount of about 30 ml were obtained for characterization from IPF patients (n=30-40) determined to meet ATS diagnostic criteria for IPF and with previous confirmation of diagnosis from an earlier lung biopsy. For certain experiments about 5 ml of whole blood is drawn into PAXgene tubes. RNA is extracted using RNA STAT-60 and treated with DNAse (Roche Molecular Biochemicals, Indianapolis, Ind.). cDNA is generated with Superscript II (Gibco/BRL) reverse transcriptase and screened by RTqPCR for expression of genes for inflammatory cytokines and chemokines in a 384-well form a using SYBR green and TaqMan assays according to the standard ABI protocol for the RT-PCR, with the following cycling conditions:

Stage 1: 50° C. 2 minutes-1 cycle

Stage 2: 95° C. 10 minutes-1 cycle

Stage 3: 95° C. 15 seconds-60° C. 1 minute-40 cycles

Stage 4: 95° C. 15 seconds—60° C. 1 minute—95° C. 15 seconds-1 cycle

25 μl volume with dissociation step added.

Gene expression will be normalized by ubiquitin levels.

For the generation of serum, about 5 ml aliquot of whole blood sample will be drawn into SST tubes for storage and further testing of biomarkers by immunoassay.

In additional experiments, about 20 ml of the whole blood samples is drawn into heparinized CPT tubes. These heparinized samples are used to isolate peripheral blood mononuclear cells (PBMCs) (typically, 10 ml of heparinized sample yields about 10-20×10⁶ cells). FACS analysis of the cell surface markers of the isolated peripheral blood mononuclear cells is performed (1×10⁵ cells/marker). Antibodies conjugated with florescent markers specific for: T cell markers (CD3, CD4, CD8, CD25, DR5), monocytes (CD14, CD64, class II), B cells (CD19, CD38, CD86), and NK cells (CD56) are included for the FACS analysis.

### Subjects

Eighteen subjects with IPF and 20 subjects without structural lung disease (control group) were enrolled in this study. Criteria for enrollment included a diagnosis of IPF according to American Thoracic Society/European Respiratory Society consensus classification (15). IPF Subjects complained of dyspnea and physical examination revealed finger clubbing and diffuse crackles. The high resolution CT showed bilateral subpleural reticular or ground glass changes. Such symptoms are typical for subjects with IPF. Subjects were gender-(male) and age-matched (61-83 years for the control group, and 47-81 years for the IPF group). For subsequent evaluation of sorted cell populations, blood was obtained from non-age, non-sex matched healthy donors for use as control/references for determination of baseline expression levels.

### Analysis of Peripheral Biomarkers of Inflammation

Whole blood was drawn from all subjects and collected in BD Diagnostics Vacutainer CPT cell separation tubes for FACS analysis, BD Vacutainer serum separation tubes for serum ELISA (Becton-Dickinson, San Jose, Calif.), and PAXgene Blood RNA System tubes (PreAnalytiX, Valencia, Calif.) for mRNA analysis.

### Flow Cytometric Analysis

PBMC's isolated from control subjects (n=20) and IPF patients (n=18) were stained with monoclonal antibodies conjugated with fluorescent dyes. Briefly, 0.2 to 0.5 million cells re-suspended in Phosphate Buffered Saline (PBS; Mediatech, Herndon, Va.) buffer with 1% Bovine Serum Albumin (Sigma, St Louis, Mo.) were added to each well of a V bottom 96 well microtiter plate (Fisher Scientific, Pittsburgh, Pa.). The cells were centrifuged for 5 min at 1000 rpm at room temperature, after which the supernatant was removed. The cells were stained, at the concentrations suggested by the manufacturer, with directly-conjugated cell surface antibodies (Becton Dickinson, San Jose, Calif.) against: CD29, CD36, CD44, CD49e, CD54 (receptors for adhesion molecules); CD13, CD14, CD64, CD86 (monocyte markers); CD2, CD3, CD4, CD8, CD25, CD69 (T cell markers); CD56 (NK cell marker); and the chemokine receptors CCR1, CCR2, CCR5, and CXCR4. A polyclonal antibody against the IL-25 receptor, IL-17RB, was obtained from R&D Systems, Minneapolis, Minn. The microtiter plate was gently vortexed, and incubated for 30 min at 4° C. The cells were then re-suspended in PBS/1% BSA buffer and pelleted at 1000 rpm for 5 min. at room temperature. After re-suspension in a 1% fixative solution of para-formaldehyde (Electron Microscopy Sciences, Ft. Washington, Pa.; freshly prepared from a stock solution of 16%), the cells were transferred to 5 ml polystyrene round bottom tubes. Data was acquired using BD LSR II flow cytometer equipped with FACS DiVa acquisition software for LSR II, version 4.1 (Becton Dickinson, San Jose, Calif.). A total of 30,000 events were recorded per sample.

For purification of IL17RB+ cells, PBMC were isolated from healthy human buffy coats. Cells were stained with a polyclonal antibody against IL-17RB (SEQ ID NO:53) (R&D Systems, Minneapolis, Minn.) at the manufacturer's suggested concentration, and IL-17RB+ and IL-17RB-cell populations were sorted using a BD FACS Aria I flow cytometer (Becton Dickinson, San Jose, Calif.).

### RNA Isolation

Aliquots were taken from the IPF and healthy whole blood samples and transferred to PAXgene Blood RNA System tubes (PreAnalytiX, Valencia, Calif.). Total RNA was isolated from the whole blood samples using the RNeasy method (Qiagen, Valencia, Calif.) according to the manufacturers' protocols. Total RNA (−5 μg) was subjected to treatment with DNase (Roche Molecular Biochemicals, Indianapolis, Ind., USA) according to manufacturer's instructions to eliminate possible genomic DNA contamination.

### Real-Time Quantitative PCR(RT-qPCR) for Gene Expression

DNase-treated total RNA was reverse-transcribed using Superscript II (Invitrogen, Carlsbad, Calif.) according to the manufacturer's instructions. Primers were designed using Primer Express software (Applied Biosystems, Foster City, Calif.) or obtained commercially from Applied Biosystems (ABI). Real-time quantitative PCR was performed on 10 ng of cDNA from each sample using either of two methods. In the first, 400 nM each of two gene-specific unlabelled primers was used in an ABI SYBR Green real-time quantitative PCR assay in an ABI 5700, 7000, 7300, 7700, or 7900 instrument. In the second method, 900 nM each of two unlabelled primers was used with 250 nM of FAM-labeled probe (Applied Biosystems) in a TaqMan real-time quantitative PCR reaction in an ABI 7000, 7300, or 7700 instrument. The absence of genomic DNA contamination was confirmed using primers that recognize the genomic region of the CD4 promoter. Ubiquitin levels were measured in a separate reaction and used to normalize the data by the Δ-Δ Ct method. Using the mean cycle threshold value for ubiquitin and the gene of interest for each sample, the equation 1.8 e (Ct ubiquitin minus Ct gene of interest)×10⁴ was used to obtain the normalized values. The following standard ABI cycling conditions and primers were used for RT-qPCR:

Stage 1: 50° C. 2 min.-1 cycle

Stage 2: 95° C. 10 min.-1 cycle

Stage 3: 95° C. 15 seconds-60° C. 1 min.-40 cycles

Stage 4: 95° C. 15 seconds-60° C. 1 min. 95° C. 15 seconds-1 cycle 25 μl volume with dissociation step added.

For group 1: CCR3 (Forward primer sequence: GGCACTTGCTCATGCACCT (SEQ ID NO: 1), and reverse primer sequence: GGATGGAGAGACAGAGCTGGTT (SEQ ID NO: 2)) and probe primer sequence: CAGATACATCCCATTCCTTCCTA (SEQ ID NO:35, CD87 v1-3 (PLAUR) (ABI assay, Hs00182181_ml), OPN v1-3 (SPP1) (ABI assay, Hs00959010_ml), LTF v1-2 (ABI assay, Hs00914330 ml), LCN2 (ABI assay, Hs00194353 ml), CD66d (CEACAM3) (ABI assay, Hs00174351_ml), EMR1, (ABI assay, Hs00892590_ml), CD16a (FCGR3A) (ABI assay, Hs02388314_ml), CD32a (FCGR2A) variants 1-2 and CD32c (FCGR2c) (ABI assay, Hs00234969 ml), CD11b variants 1-2 (ITGAM) (ABI assay, Hs00355885 ml), CD18 variants 1-2 (ITGB2) (ABI assay, Hs01051739_ml).

For group 2 IL-17RB (Forward primer sequence: TACGGTGCAGCTGACTCCATAT (SEQ ID NO: 3), and reverse primer sequence: GGCAGAGCACAACTGTTCCTT (SEQ ID NO: 4)), for IL-10 (Forward primer sequence: GAGATCTCCGAGATGCCTTCA (SEQ ID NO: 5), and reverse primer sequence: CAAGGACTCCTTTAACAACAAGTTGT (SEQ ID NO: 6)), for CD25 (IL2RA) (Forward primer sequence: AGATCCCACACGCCACATTC (SEQ ID NO: 7), and reverse primer sequence: TGCGGAAACCTCTCTTGCAT (SEQ ID NO: 8)), IL-23p19 (Forward primer sequence: GAACAACTGAGGGAACCAAACC (SEQ ID NO: 9), and reverse primer sequence: GCAGCAACAGCAGCATTACAG (SEQ ID NO: 10)), IL-15 (Forward primer sequence: TCCATCCAGTGCTACTTGTGTTTAC (SEQ ID NO: 11), and reverse primer sequence: CACTGAAACAGCCCAAAATGAA (SEQ ID NO: 12)), PDGFA variant 1 (ABI assay, Hs00236997_ml) and CD301 (Clec10a) (ABI assay, Hs00197107 ml),

### Pathway Analysis

Functional relationships between differentially expressed genes were identified using the Ingenuity Pathway Analysis (IPA) database (Ingenuity Systems, Redwood City, Calif.).

### Immunoassays

Human serum (30 μl) was diluted 4-fold in 1% BSA for the measurement of osteopontin using the Human osteopontin Duo Set ELISA Development kit (R&D Systems, Minneapolis, Minn.) according to the manufacturer's instructions. Human serum (˜20 μl) was diluted 5 fold in calibrator diluent for the ELISA measurement of urokinase-type plasminogen activator receptor (uPAR) using the Human uPAR Quantikine kit (R&D Systems, Minneapolis, Minn.). Human plasma (˜2 μl) was diluted 50-fold in diluent buffer for the measurement of lactoferrin using the Human lactoferrin ELISA kit (Hycult Biotechnology, Uden, The Netherlands) according to the manufacturer's instructions. Human serum (−0.20) was diluted 500-fold in MED buffer (PBS with 0.5% BSA, 0.05% Tween-20, 0.35M NaCl, 0.25% CHAPS and 5 mM EDTA) for the measurement of lipocalin 2 using the Human lipocalin 2 Duo Set ELISA Development kit (R&D Systems, Minneapolis, Minn.). Serum (100 μl) was used neat for the measurement of ST2 using the Human ST2 μL-1R4Duo Set ELISA Development kit (R&D Systems, Minneapolis, Minn.) according to the manufacturer's instructions.

### Statistical Analysis

Quantitative measures of gene expression changes and cell surface marker expression by FACS analysis were statistically evaluated using Splus software (Insightful Inc., Seattle, Wash.). Differences between the control and IPF groups were assessed using the Mann-Whitney unpaired t-test.

### Subjects

The mean age of the IPF subjects was 72.4 years, with a standard deviation (S.D.) of 6.5, while the mean age of the control subjects was 60.4 years with an S.D. of 8.9. Pulmonary physiology (FVC and D_{L}CO) contributed to assessment of disease severity and clinical prognosis (16, 17).

### EXAMPLES

### Example 1

### Phenotypic Analysis of Cell Surface Markers by Flow Cytometry on PBMC of control and IPF Subjects

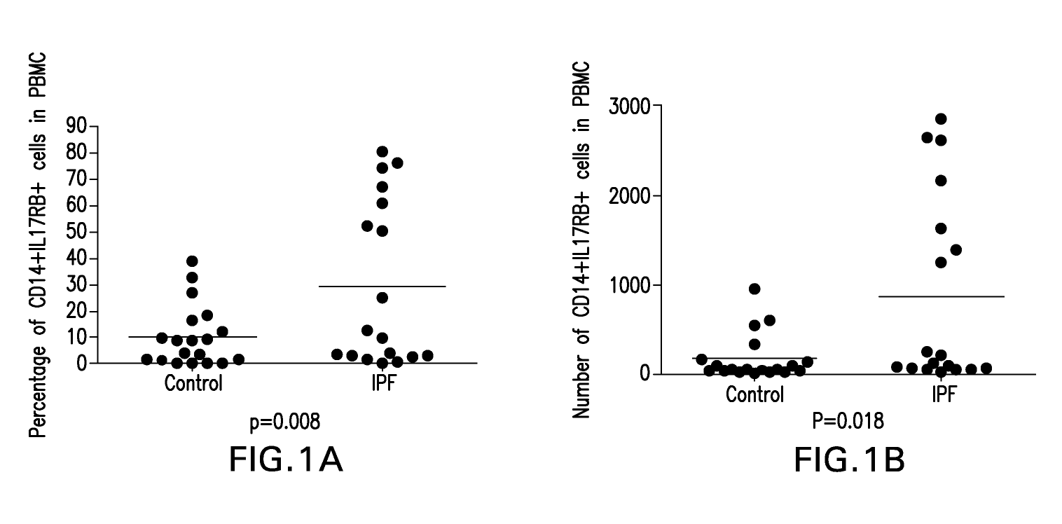

Phenotypic analysis of cell surface markers on peripheral blood cells was carried out for control and IPF subjects using multi-parametric flow cytometry as described above. The receptor for the cytokine IL-25 (IL-17RB) (SEQ ID NO:53) was found in significantly higher amounts on the monocytes/macrophage (CD14⁺ cells, SEQ ID NO:56—there are four CD14 transcript variants that all encode the same amino acid sequence) subpopulation from IPF patients (FIGS. 1A-B) than on the monocytes/macrophage (CD14⁺ cells) subpopulation from controls. FIGS. 1A-B are graphs showing expression of IL-17RB (SEQ ID NO:53) on CD14 cells in PBMC from IPF (n=18) and control (n=20) subjects. An increase in the percentage (FIG. 1A; p=0.008) and number (FIG. 1B; p=0.018) of IL-17RB+ CD14+ in IPF subjects compared to control subjects is shown.

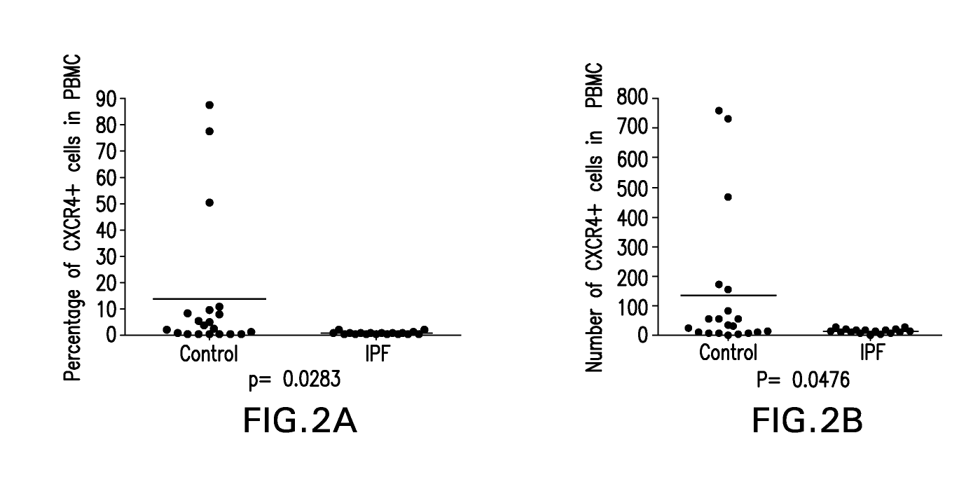

The graphs in FIGS. 2A-B show expression of CXCR4+ (SEQ ID NO:54 variant B—the probe for CXCR4 would also detect variant A SEQ ID NO:55) cells in PBMC from blood samples isolated from IPD (n=18) and control (n=20) subjects. A decrease in the CXCR4+ percent (SEQ ID NO:54, variant B or SEQ ID NO:55, variant A) (p=0.0283) and number (p=0.0476) of cells was observed in IPF patients as compared with the control subjects.

No significant difference was determined for the markers CD3, CD4, CD8 or CD56 lymphocytes in IPF patients versus control subjects.

### Example 2

### Gene Expression Analysis of Whole Blood from Control and IPF Subjects

To assess molecular changes attributable to disease status, RT-qPCR analysis of 195 selected genes was performed on RNA from whole blood samples from control (n=20) and IPF subjects (n=18). Test genes with links to inflammation, tissue remodeling, cell markers, cytokines and other chemokines of interest and their receptors, were selected for differential expression in IPF patients. Given the expected degree of variation among individuals, a nonparametric Mann-Whitney median analysis was conducted, and genes whose median levels were at least two-fold different were considered significant. Eleven genes with higher expression than control samples were detected in the IPF subjects, while seven genes had lower expression in the IPF subjects (shown in Table 1) compared to control samples.

TABLE 1
DIFFERENTIAL EXPRESSION OF GENES
IN WHOLE BLOOD OF IPF PATIENTS

| | | IPF patients vs. Control* |
|---|---|---|
| Gene Name | RefSeq Identification | patients “p Value |
| Increased in IPF | | |
| LTF variants 1-2 | NM_002343 (variant 1, SEQ | 0.001 |
| detected by primer set | ID NO: 13): lactotransferrin | |
| | (LF; HLF2; GIG12)/ | |
| | NM_001199149.1 (variant 2, | |
| | SEQ ID NO: 36) | |
| UPAR (CD87; PLAUR) | NM_001005376 (SEQ ID NO: | 0.002 |
| variants 1-3 | 14)/NM_001005377 (SEQ ID | |
| primer set detects variants | NO: 15)/NM_002659 (SEQ | |
| | ID NO: 16): plasminogen | |
| | activator, urokinase receptor | |
| | (CD87; PLAUR; UPAR; | |
| | URKR) | |
| EMR1 | NM_001974 (SEQ ID NO: | 0.004 |
| | 17): egf-like module | |
| | containing mucin-like, | |
| | hormone receptor-like 1 | |
| CD16a (FCGR3A) variant 1 | NM_000569 (SEQ ID NO: | 0.004 |
| | 18): Fc fragment of IgG, low | |
| | affinity IIIa, receptor (CD16A; | |
| | FCGR3; IGFR3; FCR-10) | |
| OPN (SPP1) variants 1-3 | NM_001040058 (variant 1, | 0.005 |
| primer set detects variants | SEQ ID NO: 19)/ | |
| | NM_001040060 (variant 2 | |
| | SEQ ID NO: 20): osteopontin; | |
| | secreted phosphoprotein 1 | |
| | (OPN; SPP1; BNSP; BSPI; | |
| | ETA-1) | |
| | NM_000582.2 (variant 3, SEQ | |
| | ID NO: 37) | |
| CCR3 variants 1-4 | NM_001837 (SEQ ID NO: | 0.009 |
| primer set detects variants | 21): chemokine (C-C motif) | |
| | receptor 3 (CCR3; CD193; | |
| | CMKBR3; CC-CKR-3) | |
| | NM_178329.2 (variant 2, | |
| | SEQ ID NO: 38) | |
| | NM_178328.1 (variant 3; | |
| | SEQ ID NO: 39) | |
| | NM_001164680.1 (variant 4, | |
| | SEQ ID NO: 40) | |
| LCN2 | NM_005564 (SEQ ID NO: | 0.009 |
| | 22); neutrophil gelatinase- | |
| | associated lipocalin; oncogene | |
| | 24p3; siderocalin (NGAL) | |
| CD11b (ITGAM) variants 1-2 | NM_000632 (variant 2, SEQ | 0.011 |
| primer set detects variants | ID NO: 23); integrin, alpha M | |
| | (complement component 3 | |
| | receptor 3 subunit) (CR3A; | |
| | MAC-1; MAC1A) | |
| | NM_001145808.1 (variant 1, | |
| | SEQ ID NO: 41) | |
| CEACAM3 (CD66D) | NM_001815 (SEQ ID NO: | 0.014 |
| | 24); carcinoembryonic | |
| | antigen-related cell adhesion | |
| | molecule 3 (CEA; CD66D) | |
| ITGB2 (CD18) variants 1-2 | NM_000211 (variant 1, SEQ | 0.019 |
| primer set detects variants | ID NO: 25): integrin beta 2 | |
| | (complement component 3 | |
| | receptor 3 and 4 subunit | |
| | (CD18; LFA-1; MAC-1) | |
| | NM_001127491.1 (variant 2, | |
| | SEQ ID NO: 42) | |
| FCGR2 (CD32a) variants 1-2 | NM_021642 (variant 2, SEQ | 0.052† |
| FCGR2 (CD32c) | ID NO: 26): Fc fragment of | |
| primer set detects variants | IgG, low affinity IIa receptor | |
| | (FCG2; FcGR; CD32A; | |
| | CDw32; IGFR2) | |
| | NM_001136219.1 variant 1 | |
| | (SEQ ID NO: 43) | |
| | NM_201563.4 (SEQ ID | |
| | NO: 44) | |
| Decreased in IPF | | |
| IL10‡ | NM_000572 (SEQ ID NO: | <0.0001‡ |
| | 27): interleukin 10 (IL10) | |
| IL-17RB (IL-25R)‡ | NM_018725 (SEQ ID NO: | 0.002‡ |
| | 28): interleukin 17 receptor B | |
| | (CRL4; EVI27; IL17BR; | |
| | IL17RH1) | |
| PDGFA variant 1‡ | NM_002607 (SEQ ID NO: | 0.002‡ |
| | 29): platelet- derived growth | |
| | factor alpha polypeptide | |
| | (PDGF1; PDGF-A) | |
| Clec10a (CD301) variants | NM_006344 (SEQ ID NO: | 0.003‡ |
| 1-2‡ | 30)/NM_182906 (SEQ ID | |
| primer set detects variants | NO: 31): C-type lectin domain | |
| | family 10, member A (HML; | |
| | HML2; CLECSF13; | |
| | CLECSF14) | |
| IL-2RA (CD25)‡ | NM_000417 (SEQ ID NO: | 0.007‡ |
| | 32): interleukin 2 receptor | |
| | alpha (CD25; IL2R; TCGFR) | |
| IL23p19‡ | NM_016584 (SEQ ID NO: | 0.012‡ |
| | 33); interleukin 23, alpha | |
| | subunit p19 (IL23A) | |
| IL-15 variants 1-3‡ | NM_000585 (variant 3, SEQ | 0.028‡ |
| primer set detects variants | ID NO: 34): interleukin 15 | |
| | (IL15) | |
| | NM_172175.2 (variant 2, | |
| | SEQ ID NO: 45) | |
| | NR_037840.1 (variant 1, non- | |
| | coding RNA, SEQ ID NO: 46) | |

*Individuals demonstrating no evidence of structural lung disease.
“p value was determined by Mann-Whitney, nonparametric T-test.
†Considered statistically significant for this study.
‡Gene expression was lower in IPF patients.

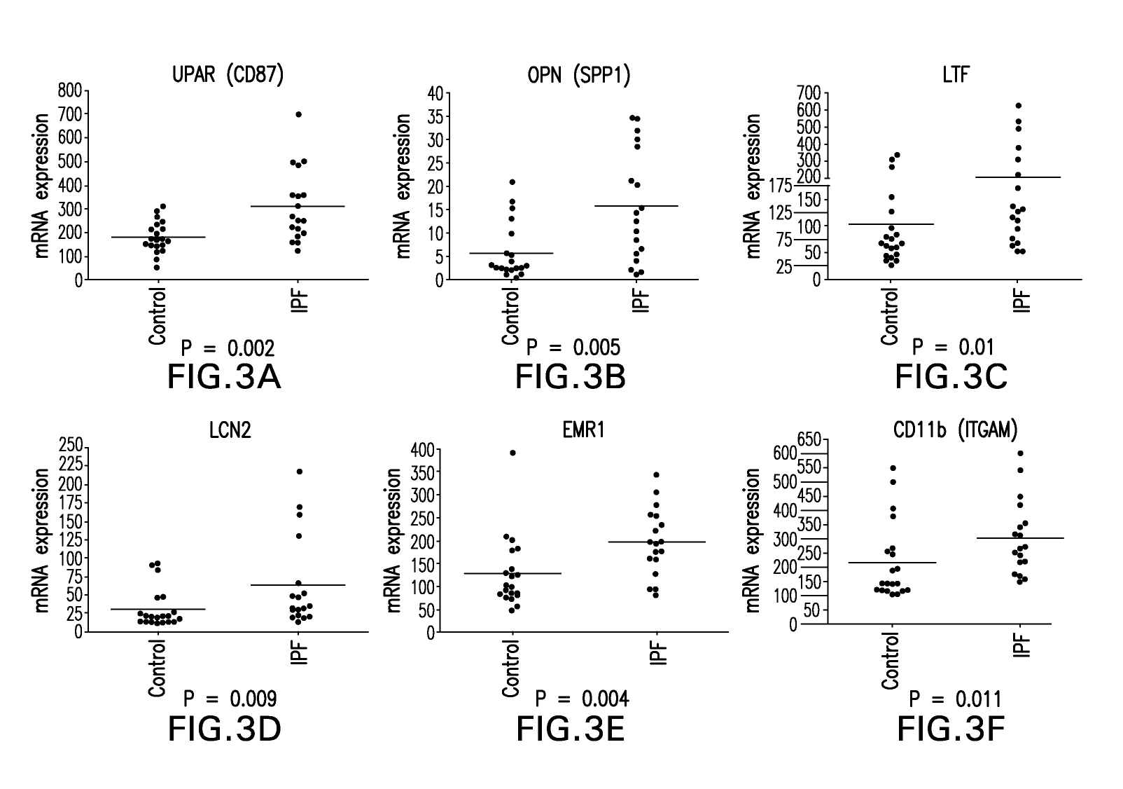

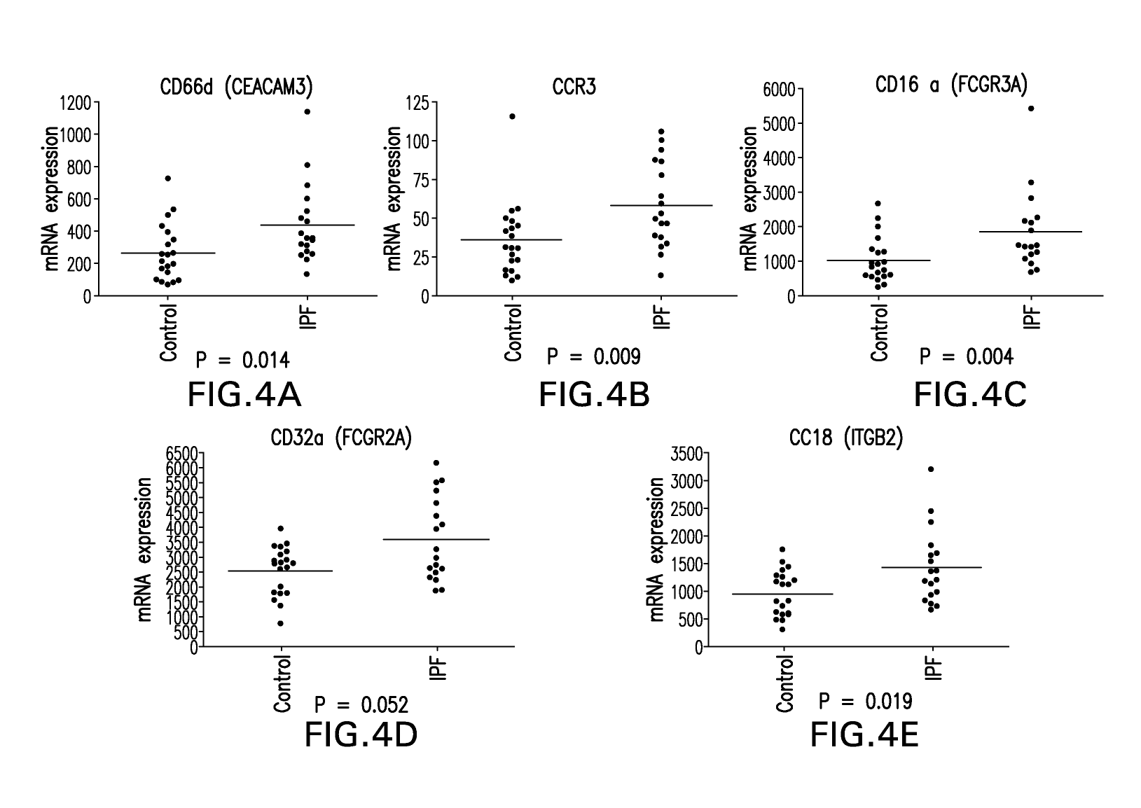

As described above in Table 1, the eleven genes found to have increased mRNA levels in the IPF patients were CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD 16a/FCGR3A variant 1 (SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42) as also shown in FIGS. 3A-F and FIG. 4A-E. The graphs in FIGS. 3A-F illustrate the differential mRNA expression as measured by RT-qPCR in the whole blood of control (n=20) and IPF (n=18) patients. Shown are increased mRNA levels of CD87 (UPAR) (SEQ ID NO:14-16) (FIG. 3A), OPN (variants 1-3: SEQ ID NO:19, SEQ ID NO:20, and SEQ ID NO:37) (FIG. 3B), LTF (variants 1-2: SEQ ID NO:13 and SEQ ID NO:36) (FIG. 3C), LCN2 (SEQ ID NO:22) (FIG. 3D), EMR1 (SEQ ID NO:17) (FIG. 3E), and CD11b (variants 1-2: SEQ ID NO:41 and SEQ ID NO:23) (ITGAM) (FIG. 3F), as examples of a disease signature observed in the whole blood of IPF patients.

The graphs in FIGS. 4A-E illustrate the differential mRNA expression as measured by RT-qPCR in the whole blood of control subjects (n=20) and IPF (n=18) patients. Shown are increased mRNA levels of CD66d (CEACAM3) (SEQ ID NO:24) (FIG. 4A), CCR3 (variants 1-4: SEQ ID NO:21, SEQ ID NO:38, SEQ ID NO:39, and SEQ ID NO:40) (FIG. 4B), CD16a (FCRGR3A) variant 1 (SEQ ID NO:18) (FIG. 4C), CD32a (FCGR2) variants 1-2 and CD32c/FCGR2c (SEQ ID NO:43 SEQ ID NO:26, and SEQ ID NO:44) (FIG. 4D), and CD18 (ITGB2) (variants 1-2: SEQ ID NO:25 and SEQ ID NO:42)(FIG. 4E), as examples of a disease signature observed in the whole blood of IPF patients.

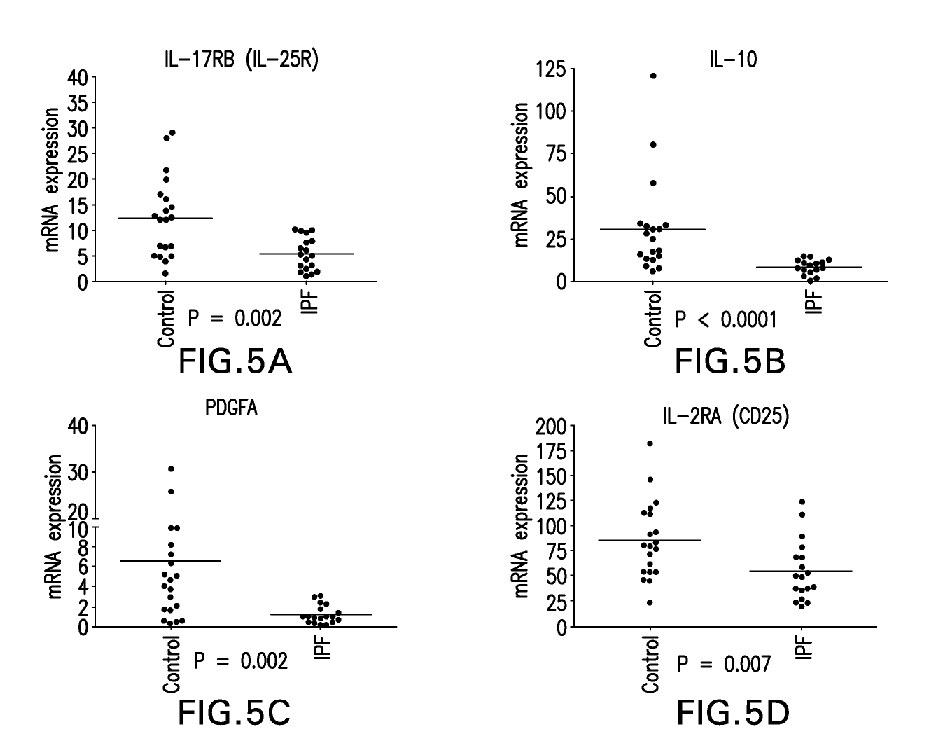

As described in Table 1, and in FIGS. 5A-D, seven genes exhibited decreased mRNA levels in the IPF patients: IL-10 (SEQ ID NO:27) (FIG. 5B), IL-17RB (IL-25R) (SEQ ID NO:28) (FIG. 5A), Clec10a (CD301) (SEQ ID NO:30/SEQ ID NO:31), IL-2RA (SEQ ID NO:32) (FIG. 5D), IL-23p19 (SEQ ID NO:33), PDGFA variant 1 (SEQ ID NO:29)(FIG. 5C), and IL-15 (variants 1-3: SEQ ID NO:46, SEQ ID NO:45 and SEQ ID NO:34).

### Example 3

### Analysis of Serum Proteins from IPF and Control Subjects

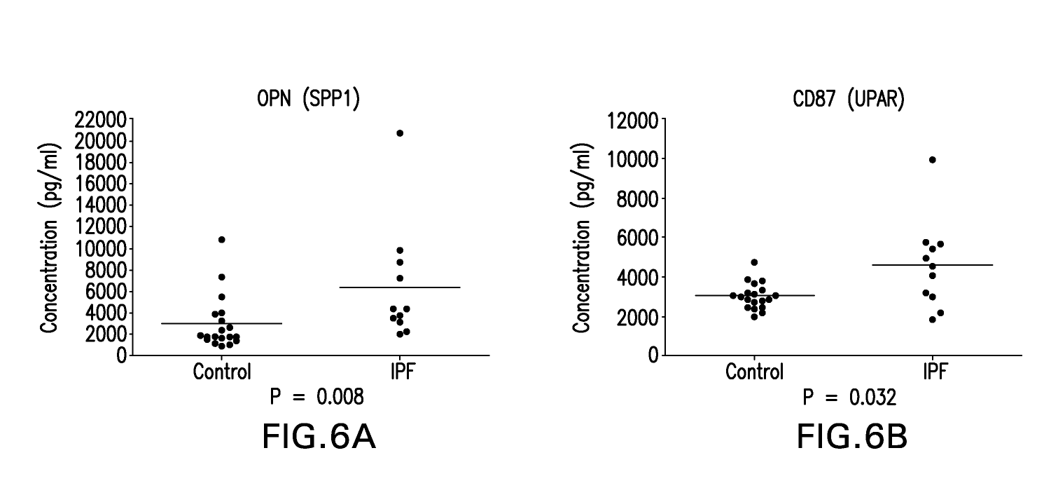

In order to correlate the observed increased gene expression levels from whole blood with protein levels in serum, a selected number of markers were analyzed for both gene expression and the corresponding protein level. ELISA Assays were performed to measure serum concentrations of OPN(SPP1) (probe detects (SEQ ID NO:47 variant B, as well as SEQ ID NO:48 variant A), LTF (probe detects SEQ ID NO:57 variant 1 and SEQ ID NO:58 variant 2), LCN2 (SEQ ID NO:59), CD87 (UPAR) (probe detects SEQ ID NO:50 variant 1, SEQ ID NO:51 variant 2, or SEQ ID NO: 52, variant 3) and soluble ST2 on eleven IPF subjects and all control subjects. Differential protein expression was measured by ELISA in the serum of controls (n=19) and IPF (n=11) patients. Of these, OPN(SPP1) and CD87 (UPAR) were elevated in IPF patients relative to the control subjects, as shown in FIGS. 6A-B, respectively. Thus, an ELISA test with antibodies specific for OPN (recognizing any of the variants SEQ ID NO:50 variant B, SEQ ID NO:48 variant A, or SEQ ID NO:49 variant C) alone or in combination with CD87 (SEQ ID NO:50 variant 1, SEQ ID NO:51 variant 2, or SEQ ID NO: 52, variant 3) would be a specific and accurate test for IPF.

### Example 4

Analysis of enriched IL-17RB+ cells from healthy human PBMC

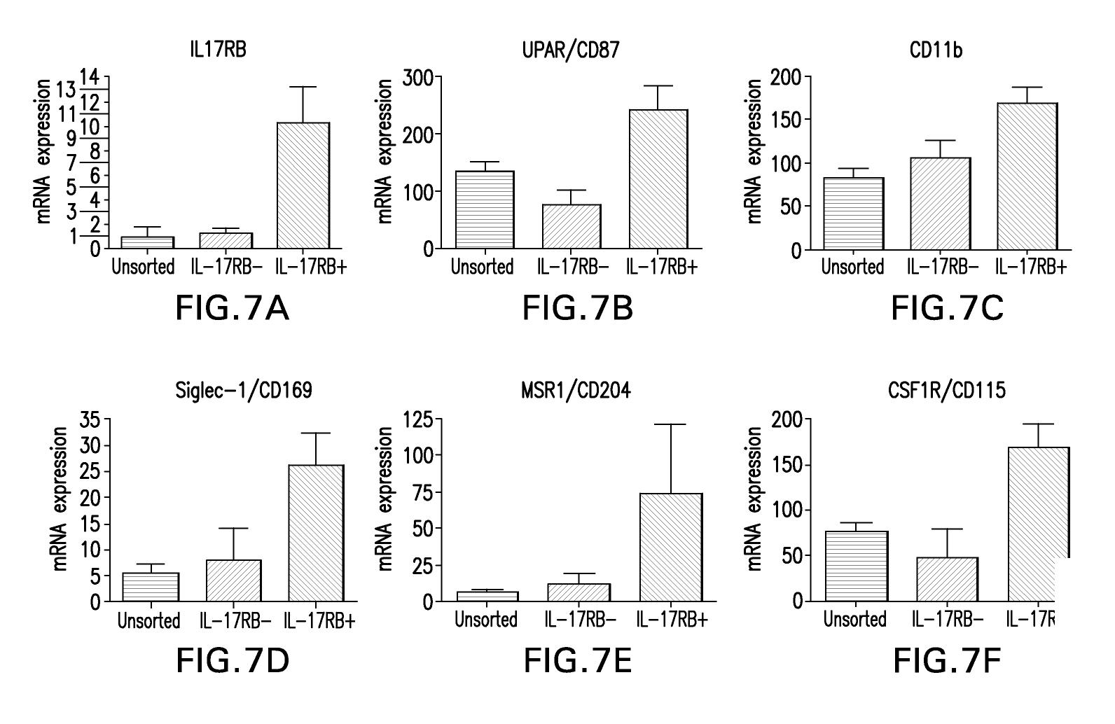

To further examine the elevated cell surface associated IL-17RB (SEQ ID NO:53) observed in IPF patients, PBMC from healthy normal donors was obtained in order to characterize these cell subsets at the molecular level. An IL-17RB+ cell population was purified from healthy human PBMC by fluorescence activated cell sorting (FACS) using a polyclonal antibody against IL-17RB (SEQ ID NO:53). Expression of IL-17RB (SEQ ID NO:28), UPAR/CD87 (SEQ ID NOs:14, 15, 16), CD11b (variants 1-2 (n=8) SEQ ID NO:41 and SEQ ID NO:23), Siglec-1/CD169 (SEQ ID NO:72), MSR1/CD204 (variants AI-AIII SEQ ID NOs:69, 73, and 74), and CSF1R/CD115 (SEQ ID NO:71) (n=4) is shown (FIGS. 7A-F). As expected, the isolated cells had higher expression of IL-17RB mRNA (SEQ ID NO:28, FIG. 7A), compared with unsorted cell populations from healthy donors. In addition, this purified cell population had elevated mRNA levels of CD87 (UPAR/PLAUR) (SEQ ID NOs:14, 15, 16) (FIG. 7B); Cd11b (variants 1-2 SEQ ID NO:41 and SEQ ID NO:23) (FIG. 7C); Siglec-1/CD169 (SEQ ID NO:72)(FIG. 7D); MSR1/CD204 (variants AI-AIII SEQ ID NOs:69, 73, and 74) (FIG. 7E); and CSF1R/CD115 (SEQ ID NO:71) (FIG. 7F) which are markers associated with monocyte/macrophage activation.

The cytokine receptor IL-17RB expressed on CD14+ cells, and associated genes CD87/UPAR(SEQ ID NOs:14, 15, 16), MSR1 (variants AI-ATH SEQ ID NOs:69, 73, and 74), CSF1R (SEQ ID NO:71) and Siglec-1 (SEQ ID NO:72), may serve as candidate cellular markers for this disease. The MSR1 (variants AI-AIII (SEQ ID NOs:69, 73, and 74))(ABI assay, Hs00234007 ml), CSF1R (SEQ ID NO:71) (ABI assay, Hs00234617_ml) and Siglec-1 (SEQ ID NO:72) (ABI assay, Hs00224991_ml) differential mRNA analysis was done on four of eight IL-17RB+ sorted cell populations from healthy donors with these 3 genes being differential in three of those sorts. These three genes, MSR1 (variants AI-AIH, SEQ ID NOs:69, 73, and 74), CSF1R (SEQ ID NO:71) and Siglec-1 (SEQ ID NO:72), were not differential in the whole blood mRNA analysis of the IPF patients. However, the 3 sorts from healthy donors that show MSR1 (variants AI-AIII, SEQ ID NOs:69, 73, and 74), CSF1R (SEQ ID NO:71) and Siglec-1 (SEQ ID NO:72) as differential in the IL-17RB+ cells also showed that the following genes had higher levels of mRNA: CD11b (variants 1-2 (ITGAM) SEQ ID NO:41 and SEQ ID NO:23), CD32a (FCGR2A) (variants 1-2 and CD32c/FCGR2c, SEQ ID NOs:43, 26, and 44), CD87 (SEQ ID NOs:14, 15, 16), CD14 (variants 1-4 (SEQ ID NO:70, SEQ ID NO:75, SEQ ID NO:76, SEQ ID NO:77), IL-17RB (SEQ ID NO:28), LCN2 (SEQ ID NO:22), EMR1 (SEQ ID NO:17), and CD66d (SEQ ID NO:24) (Ceacam3 a neutrophil marker; would suggest that IL-17RB cells were not strictly mature monocyte population). SPP1 (OPN) (variants 1-3, SEQ ID NOs:19, 20, and 37) and LTF (variants 1-2 SEQ ID NOs:13 and 36) were not detected in these 3 sorts. CD16a (variant 1 SEQ ID NO:18) and CD18 (variants 1-2 SEQ ID NOs:25 and 42) had higher levels of mRNA in 2 of the 3 sorts. IL-17RA, also an IL-25R, had higher levels of mRNA in 2 of the 3 sorts, but it is not differential in the blood of IPF patients.

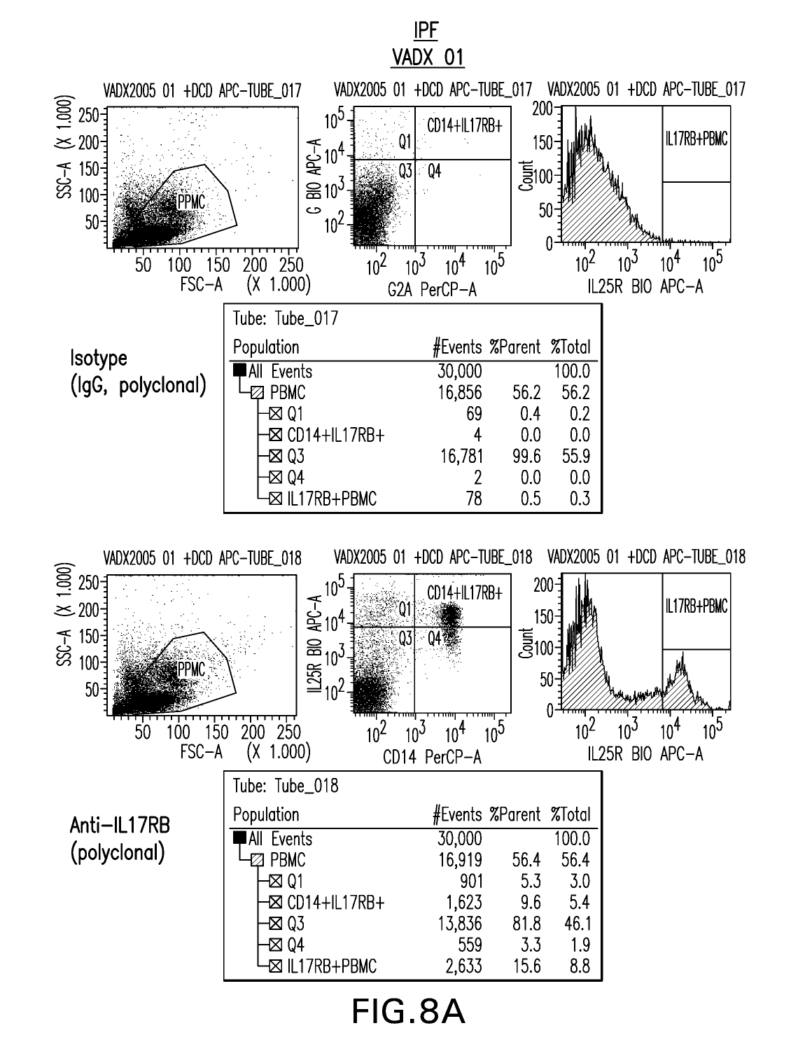

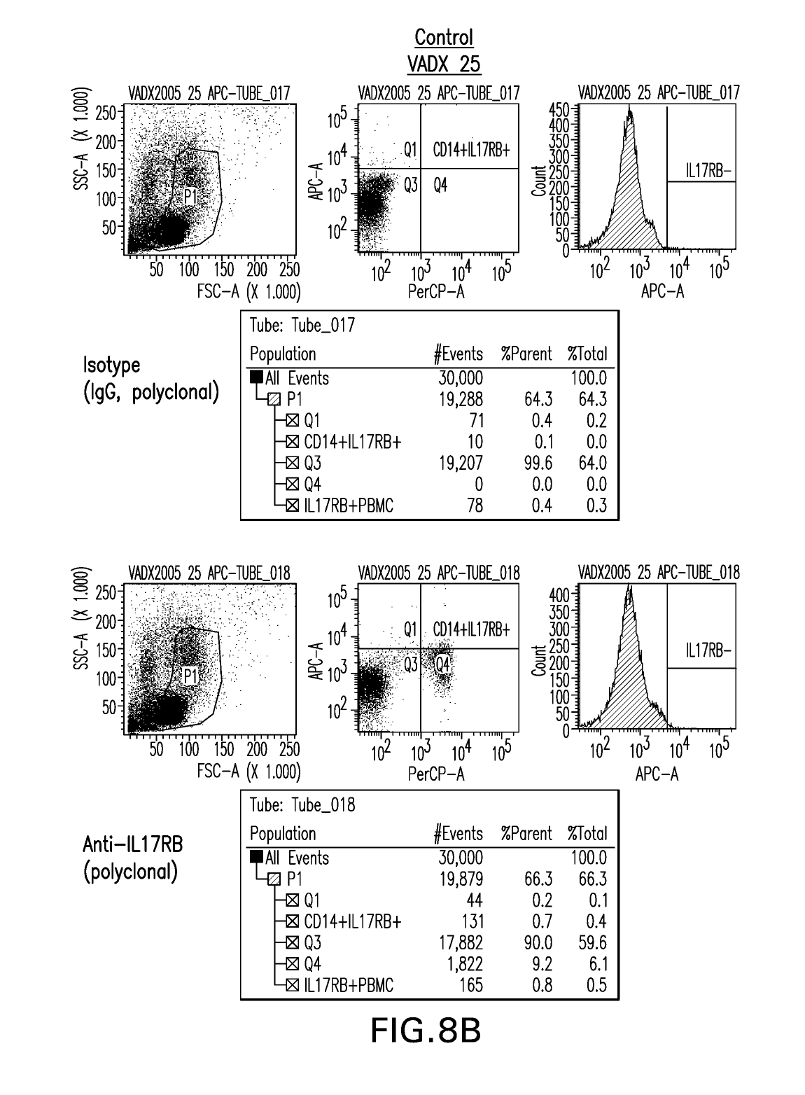

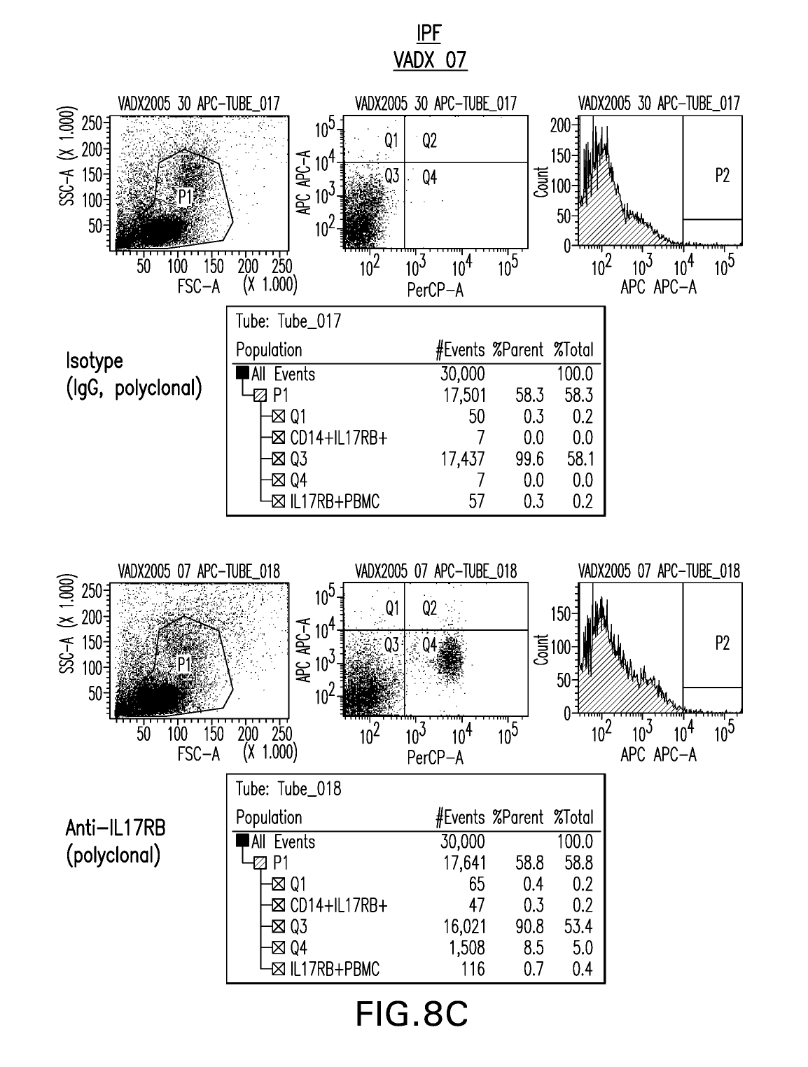

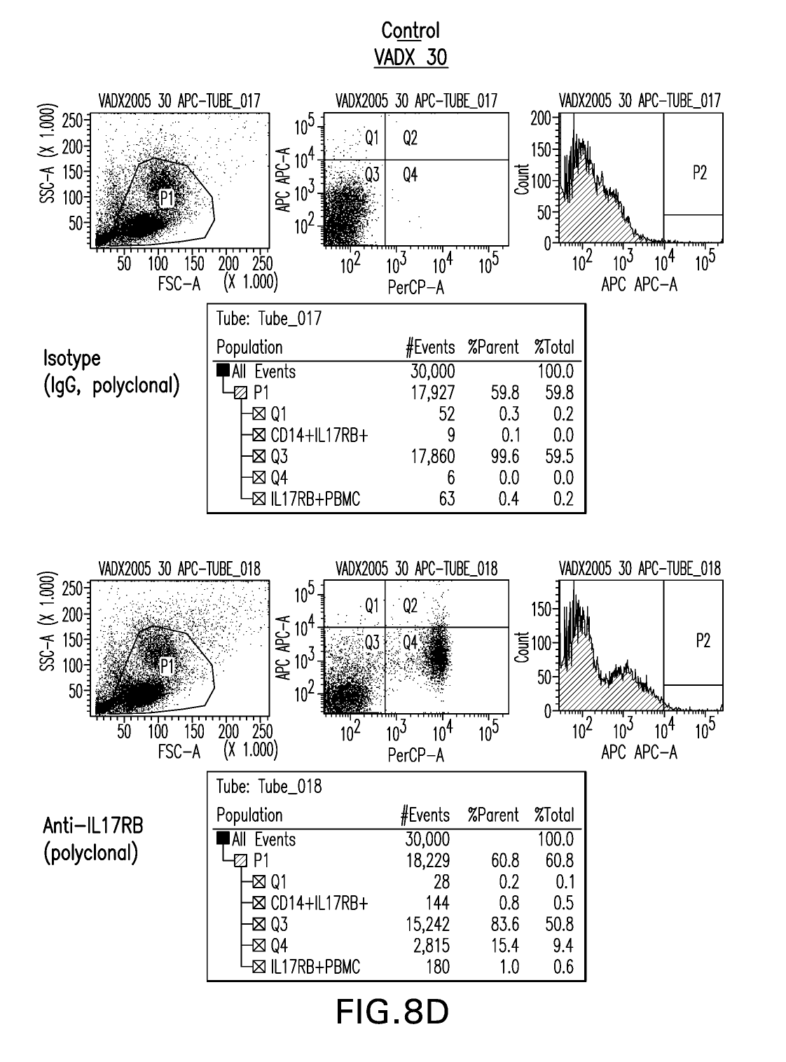

FIGS. 8A-D show representative FACS plots showing expression of IL-17RB (SEQ ID NO:53 in PBMC of two IPF patients and two control subjects. Dot plots showing percentage of CD14+IL-17RB+ cells gated on cells from PBMC gated on mononuclear cell scatter. Expression of IL-17RB (SEQ ID NO:53) was significantly higher in patient VADX 01 (FIG. 8A) compared to the control subject VADX 25 (FIG. 8B), while the expression of IL-17RB was similar in patient VADX 07 (FIG. 8C) and control VADX 30 (FIG. 8D). These data illustrate variable IL-17RB expression in IPF patients.

### CONCLUSIONS

Statistically significant changes in certain cellular and molecular markers were detected in blood samples of IPF patients when compared to control samples. Additionally, the receptor for the cytokine IL-25 (IL-17RB) (SEQ ID NO:53), was significantly higher in CD14⁺ PBMC from IPF patients. The expression of the chemokine receptor CXCR4 (probe detects both variant B SEQ ID NO:54 and variant A SEQ ID NO:55)) was lower in IPF patient PBMC.

Gene expression analyses identified 18 differentially expressed genes (and various isoforms thereof) out of a pool of 195 tested genes. Of these, CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A 9variant 1 SEQ ID NO:18), CD32aIFCGR2A (variants 1-2 and CD32c/FCGR2c SEQ ID NO:43, SEQ ID NO:26 and SEQ ID NO:44), CD11b/ITGAM (variants 1-2 SEQ ID NO:41 and SEQ ID NO:23) and CD18/ITGB2 (variants 1-2 SEQ ID NO:25 and SEQ ID NO:42) were up-regulated, while IL17RB (SEQ ID NO:28), IL10 (SEQ ID NO:27), PDGFA (variant 1 SEQ ID NO:29), CD301/Clec10a (variants 1-2: SEQ ID NO:30 and SEQ ID NO:31), CD25/IL-2RA (SEQ ID NO:32), IL23p19 (SEQ ID NO:33) and IL-15 (variants 1-3 SEQ ID NOs: 46, 45, and 34) were down-regulated in IPF samples. Differentially regulated genes were in the functional areas of inflammation and cell signaling. Additionally, the macrophage adhesion/activation proteins CD87/UPAR and OPN were analyzed by ELISA and were found to correlate with their higher gene expression level in IPF patient sera.

Purified IL-17RB+ cells from healthy human PBMC expressed monocyte/macrophage associated genes CD87/UPAR (SEQ ID NOs:14, 15, 16), CD11b variants1-2 (SEQ ID NO:41 and SEQ ID NO:23), CD18 variants 1-2 (SEQ ID NO:25 and SEQ ID NO:42) and CD16a variant 1 (SEQ ID NO: 18), indicating changes in gene expression markers in the monocyte population in IPF.

Differences in cell and molecular markers involved in monocyte/macrophage activation and migration in IPF patients were determined and identified. Thus, it is expected that a role for IL-17RB (SEQ ID NO:53) expressed on CD14+ cells in IPF is likely.

### DISCUSSION

The data obtained from these studies have revealed a number of disease specific signatures for IPF. The studies described herein were designed to detect blood related changes in IPF patients. The results have identified cellular phenotypic markers as well as gene expression profiles from peripheral blood of IPF patients. These markers will facilitate the diagnosis of IPF patients by using blood samples—which are easy to obtain and process. Tests utilizing any combination of the gene expression and phenotypic markers described herein will provide useful diagnostic tools for identification of IPF patients, providing an opportunity to initiate treatments and therapies to prevent further lung scarring.

In particular, FACS analysis was utilized to determine that an increased number of CD14+ cells (marker of monocyte/macrophage lineage) expressing the IL-25 receptor IL-17RB (SEQ ID NO:53) were found in the blood of IPF patients. While IL17RB has been reported to be expressed predominantly in CD14+ cells (11), prior to the present results, there has been no association of an increased number of CD14+ cells expressing IL-17RB in the blood of IPF patients.

Interleukin 25 is known to play an important role in augmentation of Th2-cell mediated inflammatory responses, and elevated expression of IL-25 and IL-17RB has been observed in asthmatic lung tissues (10). Gratchev et al. have reported a strong up-regulation of IL-17RB gene expression in human alternatively activated macrophages (22). Additionally, activation of alveolar macrophages by the alternative pathway has been reported in herpesvirus-induced murine model of progressive pulmonary fibrosis as well as in IPF patients (23). The elevated level of IL-17RB+/CD14+ cells described herein indicates that there is likely a role for this cell type in IPF.

Additionally, the FACS analysis showed a decrease in the percentage of PBMC expressing CXCR4 (probe detects SEQ ID NO:54 variant B and SEQ ID NO:55 variant A) in IPF patients, when compared with control PBMC samples. The CXCR4 chemokine receptor, expressed on Th2 cells, has been associated with Th2 cell-mediated allergic diseases such as asthma (12, 13). Studies have shown that blocking CXCR4 results in an inhibition of airway hyperreactivity and an overall lung inflammatory response in a mouse model of asthma (13 Lukacs et al. 2002). The CXC family chemokines are believed to be important in the pathogenesis of IPF and other fibroproliferative diseases because of their role in leukocyte trafficking, vascular remodeling, regulation of angiogenesis, and mobilization and trafficking of mesenchymal progenitor cells known as fibrocytes (14, 24). Identifying altered expression of CXC chemokines in peripheral blood provides an alternate, easier and cheaper clinical detection method compared to utilizing lung samples where CXC chemokines were initially observed in the lung environment of IPF patients (14, 25, 26).

Comparisons of control and IPF subjects by gene expression in whole blood by RT-qPCR identified a putative disease signature of 18 genes expressed differentially between control and IPF subjects. Of these, eleven genes were determined to have increased mRNA levels in the IPF patients: of CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2 SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4 SEQ ID NO:21, SEQ ID NO:38, SEQ ID NO:39, and SEQ ID NO:40), CD16a/FCGR3A variant 1 (SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2 SEQ ID NO:23, SEQ ID NO:41) and CD18/ITGB2 (variants 1-2 SEQ ID NO:25 and SEQ ID NO:42) while expression of at least one of the following genes were lower than control expression: IL17RB (SEQ ID NO:28), IL10 (SEQ ID NO:27), PDGFA variant 1 (SEQ ID NO:29), CD301/Clec10a (variants 1-2: SEQ ID NO:30 and SEQ ID NO:31), CD251IL-2RA (SEQ ID NO:32), IL23p19 (SEQ ID NO:33) and IL-15 variants 1-3 (SEQ ID NO:46, SEQ ID NO:45, and SEQ ID NO:34).

Many of these genes are expressed by multiple cell types, including human PBMC subpopulations. Most of them have been detected in human monocytes, neutrophils and eosinophils, except LTF variants (SEQ ID NOs:13 and 36), which are not expressed in monocytes, and CD16a (SEQ ID NO:18), which is not expressed in eosinophils (27-30). LCN2 (SEQ ID NO:22), has been described on macrophages (M1 & M2) by Fleetwood et al. GM-CSF- and M-CSF-dependent macrophage phenotypes display differential dependence on type I interferon signaling. (Fleetwood A J, Dinh H, Cook A D, Hertzog P J, Hamilton J A. J Leukoc Biol. 2009 August; 86(2):411-21. Epub 2009 Apr. 30.)

The elevated expression of Urokinase Receptor CD87 (UPAR) (variants 1-3: SEQ ID NO: 14, SEQ ID NO:15 and SEQ ID NO:16) in IPF patient whole blood is of particular interest because this protein is highly expressed in human macrophages and augments adhesion of human monocytes mediated through CD11b and CD18, a β2 integrin involved in adhesion (28, 31). A physical association, strongly influencing monocyte adhesion and activation, has been reported between CD87/UPAR and CD11b/CD18 or Mac-1 on monocytes (28, 32). In addition, CD87/UPAR has been shown to promote macrophage infiltration into the aortic wall of ApoE deficient mice (33). Complex formation between CD11b/CD18 and CD16a has been reported to play an important role in neutrophil adhesion, migration and activation (34-36). Interestingly, four genes involved in adhesion/migration CD87/UPAR (SEQ ID NOs:14, 15, 16), CD16a variant 1 (SEQ ID NO:18), CD11b variants 1-2 (SEQ ID NOs:41 and 23) and CD18 variants 1-2 (SEQ ID NOs:25 and 42) were up-regulated in the blood of IPF patients in this study, suggesting a possible role for these important adhesion and activation markers in IPF. Additionally, CD87/UPAR protein levels were elevated in IPF patient serum, indicating that this molecule could serve as a possible biomarker in IPF patients and also OPN(SPP1), as described below, would be useful for detection by ELISA.

Another upregulated gene that has been shown to be expressed in CD14+ monocytes, as well as eosinophils, neutrophils and T cells, was the chemokine receptor CCR3 gene (variants 1-4: SEQ ID NOs:21, 38, 39, and 40)(24, 37). CCR3 is associated with asthma, and has been shown to play a role in granulocyte recruitment and bleomycin-induced lung fibrosis (37). Treatment with CCR3-neutralizing antibodies inhibited fibrosis as well as granulocyte migration to the lung (37). The cytokine Osteopontin (OPN, also known as SPP1), another fibrogenic protein expressed mainly by activated macrophages and upregulated in our study, has been observed to have increased levels of mRNA and protein in the lung and BAL fluid of IPF patients (38). Migration of neutrophils and fibroblasts in response to OPN has also been documented (39). The present results have demonstrated elevated OPN in the serum of IPF patients, suggesting another accessible biomarker for IPF.

Other genes that were upregulated in IPF patients were EMR1 (Egf-like module containing, mucin-like, hormone receptor-like 1) (SEQ ID NO:17), LTF variants 1-2 (SEQ ID NOs:13 and 36) and LCN2 (SEQ ID NO:22), all three of which are expressed in eosinophils and neutrophils (14, 40, 41). EMR1 is a homolog of the F4/80 rat macrophage marker and is expressed on human macrophages as well as, more recently, on eosinophils (30, 41). It is homologous to the secretin family of proteins and is believed to be involved in cell adhesion and signal transduction (42). Proteins encoded by LTF and LCN2 are components of the secretary granules of neutrophils (40, 43). Increased LCN2 has been observed in the serum of cystic fibrosis patients, and correlated with decreased pulmonary function (44). Investigators have hypothesized that LCN2, a possible suppressor of angiogenesis in pancreatic cancer (45, Tong et al. 2008), may be involved in the aberrant wound healing that is observed in IPF patients (14).

One of the seven genes that had decreased mRNA levels in whole blood from IPF patients was IL-17RB (SEQ ID NO:28) (FIG. 5A). This gene expression result in whole blood does not contradict the FACS observation of increased IL-17RB (SEQ ID NO:53) in the CD14+ subpopulation of PBMC a small component of whole blood cells).

Taken together, these data illustrate that significant changes in cellular and molecular markers of inflammation and oxidative stress can be detected in blood samples from IPF patients and can serve as biomarkers for disease. The differences were in gene products with functions mainly related to monocyte/macrophage activation, migration and fibrosis. The observation that IL-17RB (SEQ ID NO:28) expression in CD14+ cells is upregulated in the blood of IPF patients is particularly interesting. Purification and characterization of blood cell populations from IPF patients, including cells expressing the IL-25 receptor IL-17RB (SEQ ID NO:53), will be important for further understanding of this complex disease.

Additionally, this enhanced expression correlates with higher levels of expression of selected genes such as CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A (variant 1 SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42). In certain preferred embodiments, the detection of higher expression of all of these genes (e.g., IPF signature, or high expression IPF signature) can serve as a biomarker for IPF and can also serve as a method for monitoring patient treatment. Furthermore, the detection of the higher expression IPF signature can be combined with detection of the lower expression signature for IPF indicated by the lower expression of IL17RB (SEQ ID NO:28), IL10 (SEQ ID NO:27), PDGFA variant 1 (SEQ ID NO:29), CD301/Clec10a (variants 1-2: SEQ ID NO:30 and SEQ ID NO:31), CD25/IL-2RA (SEQ ID NO:32), IL23p19 (SEQ ID NO:33) and IL-15 (variants 1-3: SEQ ID NO:46, SEQ ID NO:45, and SEQ ID NO:34) genes (e.g., low expression IPF signature). In certain embodiments, these genetic IPF signatures will be combined with cell surface marker expression of increased IL-17R13 (SEQ ID NO:37) expression in CD14+ cells to form an IPF diagnostic regime.

These molecular biomarkers (in any desired combination) can also be used for screening for agents that affect expression of one or more of the genes that exhibit higher levels of expression in IPF patients (i.e., that decrease expression of any one or more of CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A (variant 1 SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42). Additionally, any one or more biomarkers that exhibit a decrease in expression in IPF patients can be used for screening for agents that increase expression of any one or more of these genes (i.e., an agent that increases expression of any one of more of IL17RB (SEQ ID NO:28), IL10 (SEQ ID NO:27), PDGFA variant 1 (SEQ ID NO:29), CD301/Clec10a (variants 1-2: SEQ ID NO:30 and SEQ ID NO:31), CD25/IL-2RA (SEQ ID NO:32), IL23p19 (SEQ ID NO:33) and IL-15 (variants 1-3: SEQ ID NO:46, SEQ ID NO:45, and SEQ ID NO:34). Furthermore, agents can be tested for their affects (either increased or decreased expression as appropriate) on any combination of the molecular biomarkers for IPF including one or more of: CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A (variant 1 SEQ ID NO:18), CD32aIFCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42), and IL17RB (SEQ ID NO:28), IL10 (SEQ ID NO:27), PDGFA variant 1 (SEQ ID NO:29), CD301/Clec10a (variants 1-2: SEQ ID NO:30 and SEQ ID NO:31), CD25/IL-2RA (SEQ ID NO:32), IL23p19 (SEQ ID NO:33) and IL-15 (variants 1-3: SEQ ID NO:46, SEQ ID NO:45, and SEQ ID NO:34).

These data indicate that blood can be a convenient and reliable source for measuring potential cellular and molecular biomarkers in IPF as well as for screening for effective agents for treating IPF, and for monitoring IPF therapies and disease progress. The cytokine receptor IL-17RB (SEQ ID NO:53) expressed on CD14+ cells, and associated genes CD87/UPAR (SEQ ID NOs:14, 15, 16), MSR1 variants AI-AIIII (SEQ ID NOs:69, 73 and 74), CSF1R (SEQ ID NO:71) and Siglec-1 (SEQ ID NO:72), in certain embodiments, can serve as candidate cellular markers for IPF, alone or in combination with the biomarkers described herein.

### REFERENCES

1. Raghu G, Weycker D, Edelsberg J, Bradford W, Oster G. Incidence and prevalence of idiopathic pulmonary fibrosis. AJRCCM 2006; 174:810-6.
2. Hunninghake G W and Schwarz M L Does Current Knowledge Explain the Pathogenesis of Idiopathic Pulmonary Fibrosis? A Perspective. Proc Am Thorac Soc 2007; 4:449-452.
3. Selman M, King T E, Pardo A. Idiopathic pulmonary fibrosis:prevailing and evolving hypotheses about its pathogenesis and implications for therapy. Ann Int Med 2001; 134(2):136-151.
4. Campbell D A, Poulter L W, Janossey G et al. Immunohistological analysis of lung tissue from patients with cryptogenic fibrosing alveolitis suggesting local expression of immune hypersensitivity. Thorax 1985; 40:405-411.
5. Haslam P L. Evaluation of alveolitis by studies of lung biopsies. Lung 1990; 168:984-992.
6. Tajima S, Oshikawa K, Tominaga S, Sugiyama Y. The increase in serum soluble ST2 protein upon acute exacerbation of idiopathic pulmonary fibrosis. Chest 2003; 124:1206-1214.
7. Kamp D W. Idiopathic pulmonary fibrosis:the inflammation hypothesis revisited. Chest 2003; 1187-1190.
8. Reynolds H Y. Lung inflammation and finrosis:an alveolar macrophage-centered perspective from the 19702 to 1980s. Am J Grit Care Med 2005; 171:98-102.
9. Keane M P, Streiter R M. The importance of balanced proinflammatory and anti-inflammatory mechanisms in diffuse lung disease. Respir Res 2002; 3:5-13.
10. Wang Y-H, Angkasekwinai P, Lu N, Voo K S, Arima K, Hanabuchi 5, Hippe A, Corrigan C J, Dong C, Homey B, Yao Z, Ying S, Huston D P, Liu IL-25 augments type 2 immune responses by enhancing the expansion and functions of TSLP-DC-activated Th2 memory cells. J Exp Med 2007; 204(8):1837-1847.
11. Roberta Caruso, Carmine Stolfi, Massimiliano Sarra, Angelamaria Rizzo, Massimo Claudio Fantini, Francesco Pallone, Thomas T MacDonald, Giovanni Monteleone. Inhibition of monocyte-derived inflammatory cytokines by IL-25 occurs via p38 MAP kinase-dependent induction of SOCS-3. Blood 2009; 113(15): 3512-3519.
12. Lukacs N W, Schaller M. Lymphocyte trafficking and chemokine receptors during pulmonary disease. In: Badolato R, Sozzani S, editors. Lymphocyte trafficking in Health and disease. Basel, Switzerland: Birkhauser Verlag; 2006. p115-131.
13. Lukacs N W, Berlin A, Schols D, Skeiji R T, Bridger G J. AMD3100, a CXCR4 antagonist, attenuates allergic lung inflammation and airway hypersensitivity. Am J Pathol 2002; 160 (4): 1353-1360
14. Streiter R M, Gomperts B N, Keane M P. The role of CXC chemokines in pulmonary fibrosis. J Clin Invest 2007; 117:549-556
15. American Thoracic Society/European Respiratory Society International Multidisciplinary Consensus Classification of the Idiopathic Interstitial Pneumonias. Am J Respir Crit. Care Med 2002; 165:277-304
16. Katzenstein A L, Myers J L. Idiopathic pulmonary fibrosis: clinical relevance of pathologic classification. State of the art. Am J Respir Crit. Care Med 1998; 157: 1301-1315.
17. Ryu J H; Colby T V; Hartman T E. Idiopathic pulmonary fibrosis: current concepts. Mayo Clin Proc. 1998; 73:1085-1101.
18 Selman M, Pardo A, Barera L, Estrada A, Watson S R, Wilson K, Aziz N, Kaminski N, Zlotnik A. Gene expression profiles distinguish idiopathic pulmonary fibrosis from hypersensitivity pneumonitis. Am J Respir Crit. Care Med 2005; 173:188-198.2006.
19. Gruber R, Pforte A, Beer B, Riethmüller G. Determination of gamma/delta and other T-lymphocyte subsets in bronchoalveolar lavage fluid and peripheral blood from patients with sarcoidosis and idiopathic fibrosis of the lung. APMIS 1996; 104(3):199-205.
20. Tsoutsou P G, Gourgoulianis K I, Petinaki E, Germenis A, Tsoutsou A G, Mpaka M, Efremidou S, Molyvdas P A. Cytokine levels in the sera of patients with idiopathic pulmonary fibrosis. Respir Med. 2006; 100(5):938-945.
Rosas I O, Richards T J, Konishi K, Zhang Y, Gibson K, Lokshin A E, Lindell K O, Cisneros J, Macdonald S D, Pardo A, Sciurba F, Dauber J, Selman M, Gochuico B R, Kaminski N. MMPI and MMPI as potential peripheral blood biomarkers in idiopathic pulmonary fibrosis. PLoS Med. 2008; 5(4): e93:623-633.
22. Gratchev A, Kzhyshkowska J, Kanookadan S, Ochsentreiter M, Popova A, Yu X, Mamidi S, Stone-house-Usselmann E, Muller-Molinet I, Gooi L, and Goerdt S. Activation of a TGF-b-specific multistep gene expression program in mature macrophage requires glucocorticoid-mediated surface expression of TGF-b receptor II. J Immunol 2008; 180:6553-6565.
23. Mora A L, Tones-Gonzalez E, Rojas M, Corredor C, Ritzenthaler J, Xu J, Roman J, Brigham K, and Stecenko A. Activation of alveolar macrophages via the alternative pathway in herpesvirus-induced lung fibrosis. Am J Respir Cell Mol Biol 2006; 35:466-473.
24. Phillips R J, Burdick M D, Hong K, Lutz M A, Murray L A, Xue Y Y, Belperio J A, Keane M P, Strieter R M. J Clin Invest 2004; 114:438-446
25. M. P. Keane The role of chemokines and cytokines in lung fibrosis. Eur Resp Rev. 2008; 17: 151-156.
26. Antoniou K M, Tzouvelekis A, Alexandrakis M G, Sfiridaki K, Tsiligianni 1, Rachiotis G, Tzanakis N, Bouros D, Milic-Emili J, M. Siafakas N M, Different Angiogenic Activity in Pulmonary Sarcoidosis and Idiopathic Pulmonary Fibrosis Chest. 2006; 130:982-988
27. Nissinen R, Leirisalo-Repo M, Peltomaa R, Palosuo T, Vaarala O. Cytokine and chemokine receptor profile of peripheral blood mononuclear cells during treatment with infliximab inpatients with active rheumatoid arthritis. Ann Rheum Dis. 2004; 63(6):681-687.
28. Sitrin R G, Todd R F 3rd, Albrecht E, Gyetko M R. The urokinase receptor (CD87) facilitates CD11b/CD18-mediated adhesion of human monocytes. J Clin Invest. 1996; 97(8):1942-1951.
29. Preynat-Seauve O, Villiers C L, Jourdan G, Richard M J, Plumas J, Favier A, Marche P N, Favrot M C. An interaction between CD16 and CR3 enhances iC3b binding to CR3 but is lost during differentiation of monocytes into dendritic cells. Eur J. Immunol. 2004; 34(1):147-155.
30. Hamann J, Koning N, Pouwels W, Ulfman L H, van Eijk M, Stacey M, Lin H H, Gordon S, Kwakkenbos M J. EMR1, the human homolog of F4/80, is an eosinophil-specific receptor. Eur J. Immunol. 2007; 37(10):2797-802.
31. Svensson P-A, Olson F J, Hagg D A, Ryndel M, Wiklund O, Karlstrom L, Hulthe J, Carlsson L, and Fagerberg B. Urokinase-type plasminogen activator receptor is associated with macrophages and plaque rupture in symptomatic carotid atherosclerosis. Int J Mol Med 2008; 22:459-464.
32. Gyetko M R, Todd R F 3^{rd}, Wilkinson C C, Sitrin R G. The urokinase receptor is required for human monocyte chemotaxis in vitro. M R Gyetko, R F Todd, 3rd, C C Wilkinson and R G Sitrin J. Clin. Invest. 1994; 93(4): 1380-1387
33. Gu J-M, Johns A, Morser J, Dole W P, Greaves D R, Deng G G. Urokinase plasminogen activator receptor promotes macrophage infiltration into the vascular wall of ApoE deficient mice. J Cell Physiol 2005; 204:73-82
34. Pluskota E, Soloviev D A, Plow E F. Convergence of the adhesive and fibrinolytic systems:recognition of urokinase by integrin alpha Mbeta 2 as well as by the urokinase receptor regulates cell adhesion and migration. Blood 2003; 101(4):1582-1590.
35. Jakus Z, Berton G, Ligeti E, Lowell C A, Mócsai A. Responses of neutrophils to anti-integrin antibodies depends on costimulation through low affinity Fc gamma R5: full activation requires both integrin and nonintegrin signals. J Immunol. 2004; 173(3):2068-2077.
Shashidharamurthy R, Hennigar R A, Fuchs S, Palaniswami P, Sherman M, Selvaraj P. Extravasations and emigration of neutrophils to the inflammatory site depend on the interaction of immune-complex with Fcgamma receptors and can be effectively blocked by decoy Fcgamma receptors. Blood. 2008; 111(2):894-904.
Huaux F, Gharaee-Kermani M, Liu T, Morel V, McGarry B, Ullenbruch M, Kunkel S L, Wang J, Xing Z, Phan S H. Role of Eotaxin-1 (CCL11) and CC Chemokine Receptor 3 (CCR3) in bleomycin-induced lung injury and fibrosis. Am J Pathol 2005; 167:1485-1496.
38. Pardo A, Gibson K, Cisneros J, Richards T J, Yang Y, Becerril C, Yousem S, Herrera I, Ruiz V, Selman M, Kaminski N. Up-regulation and profibrotic role of osteopontin in human idiopathic pulmonary fibrosis. PLoS Med. 2005; 2(9):e251; 891-903.
39. Koh A, da Silva A P, Bansal A K, Bansal M, Sun C, Lee H, Glogauer M, Sodek J, Zohar. Role of osteopontin in neutrophil function. Immunology. 2007; 122(4):466-475.
40. Zimecki M, Stepniak D, Szynol A, Kruzel M L. Lactoferrin regulates proliferative response of human peripheral blood mononuclear cells to phytohrnagglutinin and mixed lymphocyte reaction. Arch Immunol Ther Exp 2001; 49:147-154.
41. Khazen W, M'bika J P, Tomkiewicz C, Benelli C, Chany C, Achour A, Forest C. Expression of macrophage-selective markers in human and rodent adipocytes. FEBS Lett. 2005; 579(25):5631-5634
42. Taylor P R, Martinez-Pomares L, Stacey M, Lin H-H, Brown G D, and Gordon S. Macrophage receptors and immune recognition. Annu. Rev. Immunol. 2005; 23:901-944.
43. Borregaard N, Sorensen O E, Theilgaard-Monch K. Neutrophil granules: a library of innate immunity proteins. Trends in Immunol 2007; 28(8):340-345.
44. Eichler I, Nilsson M, Rath R, Enander I, Venge P, Koller D Y. Human neutrophil lipocalin, a highly specific marker for acute exacerbation in cystic fibrosis. Eur Respir J. 1999; 14(5):1145-1149.
45. Tong Z, Ajaikumar B, Kunnurnakkara A B, Wang H, Matsuo Y, Diagaradjane P, Harikumar K B, Ramachandran V, Sung B, Chakraborty A, Bresalier R S, Logsdon C, Aggarwal B B, Krishnan S, and Guha S, Neutrophil gelatinase-associated lipocalin: A novel suppressor of invasion and angiogenesis in pancreatic cancer. Cancer Res 2008; 68:6100-6108.
46. Martinez F O, Helming L, and Gordon S. Alternative activation of macrophages: an immunologic perspective. Ann Rev Immunol 2009; 27:451-483.
47. Jacquel A, Benikhelf N, Paggetti J, Lalaoui N, Guery L, Dufour E K, Ciudad M, Racoeur C, Micheau O, Delva L, Droin N, and Solary E. Colony-stimulating factor-1-induced oscillations in phosphatidylinositol-3 Idnase/AKT are required for caspase activation in monocytes undergoing differentiation into macrophages. Blood 2009; 114:3633-3641.
48. York M R, Nagai T, Mangini A J, Lemaire R, van Seventer J M, and Lafyatis R. A macrophage marker, Siglec-1, is increased on circulating monocytes in patients with systemic sclerosis and induced by type I interferons and toll-like receptor agonists. Arthritis Rheum 2007; 56(5): 1675.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

While the compositions and methods of this invention have been described in terms of specific embodiments, it will be apparent to those of skill in the art that variations may be applied to the compositions and methods and in the steps or in the sequence of steps of the method described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope of the invention as defined by the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. A method of diagnosing idiopathic pulmonary fibrosis (IPF) in a mammalian subject, comprising determining that the level of expression of at least one nucleic acid selected from the group consisting of CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A (variant 1 SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42) in a test sample obtained from said subject is higher relative to the level of expression of the at least one nucleic acid in a control, wherein said higher level of expression is indicative of the presence of IPF in the subject from which the test sample was obtained.

2. The method of claim 1, wherein the level of expression of nucleic acids CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A (variant 1 SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42) in a test sample obtained from said subject is higher relative to the level of expression of the at least one nucleic acid in a control.

3. The method of claim 1, further comprising determining the level of expression of at least one nucleic acid selected from the group consisting of IL17RB (SEQ ID NO:28), IL10 (SEQ ID NO:27), PDGFA variant 1 (SEQ ID NO:29), CD301/Clec10a (variants 1-2: SEQ ID NO:30 and SEQ ID NO:31), CD25/IL-2RA (SEQ ID NO:32), IL23p19 (SEQ ID NO:33) and IL-15 (variants 1-3: SEQ ID NO:46, SEQ ID NO:45, and SEQ ID NO:34), in a test sample obtained from said subject is lower relative to the level of expression of the at least one nucleic acid in a control, wherein said lower level of expression is indicative of the presence of IPF in the subject from which the sample was obtained.

4. A method of diagnosing idiopathic pulmonary fibrosis (IPF) in a mammalian subject, comprising determining the level of expression of at least one nucleic acid selected from the group consisting of IL17RB (SEQ ID NO:28), IL10 (SEQ ID NO:27), PDGFA variant 1 (SEQ ID NO:29), CD301/Clec10a (variants 1-2: SEQ ID NO:30 and SEQ ID NO:31), CD25/IL-2RA (SEQ ID NO:32), IL23p19 (SEQ ID NO:33) and IL-15 (variants 1-3: SEQ ID NO:46, SEQ ID NO:45, and SEQ ID NO:34), in a test sample obtained from said subject is lower relative to the level of expression of the at least one nucleic acid in a control, wherein said lower level of expression is indicative of the presence of IPF in the subject from which the sample was obtained.

5. The method of claim 4, wherein the level of expression of nucleic acids IL17RB (SEQ ID NO:28), IL10 (SEQ ID NO:27), PDGFA variant 1 (SEQ ID NO:29), CD301/Clec10a (variants 1-2: SEQ ID NO:30 and SEQ ID NO:31), CD25/IL-2RA (SEQ ID NO:32), IL23p19 (SEQ ID NO:33) and IL-15 (variants 1-3: SEQ ID NO:46, SEQ ID NO:45, and SEQ ID NO:34) in a test sample obtained from said subject is lower relative to the level of expression of the at least one nucleic acid in a control.

6. The method of claim 4, further comprising determining that the level of expression of at least one nucleic acid selected from the group consisting of CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A (variant 1 SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42) in a test sample obtained from said subject is higher relative to the level of expression of the at least one nucleic acid in a control, wherein said higher level of expression is indicative of the presence of IPF in the subject from which the test sample was obtained

7. A method of diagnosing idiopathic pulmonary fibrosis (IPF) in a mammalian subject, comprising determining the level of expression of IL17RB in PBMC's from a test sample obtained from said subject is higher relative to the level of expression of IL17RB in PBMC's from a control, wherein said higher level of expression is indicative of the presence of IPF in the subject from which the test sample was obtained.

8. The method of any one of claims 1-7, wherein said mammalian subject is a human patient.

9. The method of any one of claims 1-7, wherein said test sample is a whole blood sample.

10. The method of any one of claims 1-6, wherein said expression level is determined by a gene expression profiling method.

11. The method of claim 10, wherein said method is a PCR-based method.

12. The method of claim 7, wherein said method is a flow cytometry-based method.

13. A method of treating idiopathic pulmonary fibrosis (IPF) in a mammalian subject in need thereof, the method comprising the steps of:
a) determining that the level of expression of at least one nucleic acid selected from the group consisting of CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A (variant 1 SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42) in a test sample obtained from said subject is higher relative to the level of expression in a control, wherein said higher level of expression is indicative of the presence of IPF in the subject from which the sample was obtained; and
b) administering to said subject an effective amount of an IPF therapeutic agent.

14. A method of treating idiopathic pulmonary fibrosis (IPF) in a mammalian subject comprising:
a) measuring expression of at least one nucleic acid selected from the group consisting of CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A (variant 1 SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42) in a blood sample from said subject;
b) determining that said subject exhibits at least about 2-fold higher expression of the at least one nucleic acid, or any combination thereof, compared to the expression in a normal blood sample, and
c) administering to said subject an effective amount of an IPF therapeutic agent.

15. An isolated plurality of genes selected from the group consisting of CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A (variant 1 SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42).

16. An isolated plurality of genes selected from the group consisting of IL17RB (SEQ ID NO:28), IL10 (SEQ ID NO:27), PDGFA variant 1 (SEQ ID NO:29), CD301/Clec10a (variants 1-2: SEQ ID NO:30 and SEQ ID NO:31), CD25/IL-2RA (SEQ ID NO:32), IL23p19 (SEQ ID NO:33) and IL-15 (variants 1-3: SEQ ID NO:46, SEQ ID NO:45, and SEQ ID NO:34).

17. An isolated plurality of genes comprising a first group and a second group of genes, wherein said first group comprises genes selected from the group consisting of CD87/UPAR (variants 1-3: SEQ ID NO: 14, SEQ ID NO:15 and SEQ ID NO:16), OPN (variants 1-3: SEQ ID NO:19, SEQ ID NO:20, and SEQ ID NO:37), LTF (variants 1-2: SEQ ID NO:13 and SEQ ID NO:36), LCN2 (SEQ ID NO:22), CEACAM3/CD66d (SEQ ID NO:24), EMR1 (SEQ ID NO:17), CCR3 (variants 1-4: SEQ ID NO:21, SEQ ID NO:38, SEQ ID NO:39, and SEQ ID NO:40), CD16a/FCGR3A (variant 1 SEQ ID NO:18), CD32a/FCGR2A variants 1-2 & CD32c/FCGR2c (SEQ ID NO:43, SEQ ID NO:26, and SEQ ID NO:44), CD11b/ITGAM (variants 1-2: SEQ ID NO:41, SEQ ID NO:23) and CD18/ITGB2 (variants 1-2: SEQ ID NO:25 and SEQ ID NO:42),
and said second group comprises genes selected from the group consisting of IL17RB (SEQ ID NO:28), IL10 (SEQ ID NO:27), PDGFA variant 1 (SEQ ID NO:29), CD301/Clec10a (variants 1-2: SEQ ID NO:30 and SEQ ID NO:31), CD25/IL-2RA (SEQ ID NO:32), IL23p19 (SEQ ID NO:33) and IL-15 (variants 1-3: SEQ ID NO:46, SEQ ID NO:45, and SEQ ID NO:34).

18. The isolated plurality of genes of claim 17, wherein the first group of genes is differentially expressed at a higher level in a test sample obtained from a mammalian subject relative to the level of expression in a control, wherein said higher level of expression is indicative of the presence of IPF in the subject from which the test sample was obtained, and wherein each gene in said second group is differentially expressed at a lower level in a test sample obtained from the mammalian subject relative to the level of expression in a control, wherein said lower level of expression is indicative of the presence of IPF in the subject from which the test sample was obtained.

19. A kit comprising the plurality of genes of any one of claims 15-18.
